SEQUENCE LISTING (49 sequences)

SEQ ID NO: 1 - DNA, 66 bp, Artificial Sequence
  pBPS_fwr_1
  misc_feature (34)..(41): n is a, c, g, or t
acactctttccctacacgacgctcttccgatctnnnnnnnncgatgtttcggttagagcggatgtg

SEQ ID NO: 2 - DNA, 66 bp, Artificial Sequence
  pBPS_fwar_2
  misc_feature (34)..(41): n is a, c, g, or t
acactctttccctacacgacgctcttccgatctnnnnnnnnacagtgttcggttagagcggatgtg

SEQ ID NO: 3 - DNA, 66 bp, Artificial Sequence
  pBPS_fwr_3
  misc_feature (34)..(41): n is a, c, g, or t
acactctttccctacacgacgctcttccgatctnnnnnnnntgaccattcggttagagcggatgtg

SEQ ID NO: 4 - DNA, 66 bp, Artificial Sequence
  pBPS_fwr_4
  misc_feature (34)..(41): n is a, c, g, or t
acactctttccctacacgacgctcttccgatctnnnnnnnngccaatttcggttagagcggatgtg

SEQ ID NO: 5 - DNA, 66 bp, Artificial Sequence
  pBPS_fwr_5
  misc_feature (34)..(41): n is a, c, g, or t
acactctttccctacacgacgctcttccgatctnnnnnnnnatcacgttcggttagagcggatgtg

SEQ ID NO: 6 - DNA, 66 bp, Artificial Sequence
  pBPS_fwr_6
  misc_feature (34)..(41): n is a, c, g, or t
acactctttccctacacgacgctcttccgatctnnnnnnnnggctacttcggttagagcggatgtg

SEQ ID NO: 7 - DNA, 66 bp, Artificial Sequence
  pBPS_fwr7
  misc_feature (34)..(41): n is a, c, g, or t
acactctttccctacacgacgctcttccgatctnnnnnnnntagcttttcggttagagcggatgtg

SEQ ID NO: 8 - DNA, 66 bp, Artificial Sequence
  pBPS_fwr_8
  misc_feature (34)..(41): n is a, c, g, or t
acactctttccctacacgacgctcttccgatctnnnnnnnntctcccttcggttagagcggatgtg

SEQ ID NO: 9 - DNA, 70 bp, Artificial Sequence
  pBPS_rev_1
  misc_feature (34)..(41): n is a, c, g, or t
gtgactggagttcagacgtgtgctcttccgatcnnnnnnnntatatacgcaggtaacccatatgcatggc

SEQ ID NO: 10 - DNA, 70 bp, Artificial Sequence
  pBPS_rev_2
  misc_feature (34)..(41): n is a, c, g, or t
gtgactggagttcagacgtgtgctcttccgatcnnnnnnnncgctctatcaggtaacccatatgcatggc

SEQ ID NO: 11 - DNA, 70 bp, Artificial Sequence
  pBPS_rev_3
  misc_feature (34)..(41): n is a, c, g, or t
gtgactggagttcagacgtgtgctcttccgatcnnnnnnnngagacgtctaggtaacccatatgcatggc

SEQ ID NO: 12 - DNA, 70 bp, Artificial Sequence
  pBPS_rev_4
  misc_feature (34)..(41): n is a, c, g, or t
gtgactggagttcagacgtgtgctcttccgatcnnnnnnnnatactgcgtaggtaacccatatgcatggc

SEQ ID NO: 13 - DNA, 70 bp, Artificial Sequence
  pBPS_rev5
  misc_feature (34)..(41): n is a, c, g, or t
gtgactggagttcagacgtgtgctcttccgatcnnnnnnnnactagcagaaggtaacccatatgcatggc

SEQ ID NO: 14 - DNA, 70 bp, Artificial Sequence
  pBPS_rev_6
  misc_feature (34)..(41): n is a, c, g, or t
gtgactggagttcagacgtgtgctcttccgatcnnnnnnnnctgctactcaggtaacccatatgcatggc

SEQ ID NO: 15 - DNA, 70 bp, Artificial Sequence
  pBPS_rev7
  misc_feature (34)..(41): n is a, c, g, or t
gtgactggagttcagacgtgtgctcttccgatcnnnnnnnnccgtacacaaggtaacccatatgcatggc

SEQ ID NO: 16 - DNA, 69 bp, Artificial Sequence
  pBPS_rev8
  misc_feature (34)..(41): n is a, c, g, or t
gtgactggagttcagacgtgtgctcttccgatcnnnnnnnnatctgcaaaggtaacccatatgcatggc

SEQ ID NO: 17 - DNA, 70 bp, Artificial Sequence
  D501
aatgatacggcgaccaccgagatctacactatagcctacactctttccctacacgacgctcttccgatct

SEQ ID NO: 18 - DNA, 70 bp, Artificial Sequence
  D502
aatgatacggcgaccaccgagatctacacatagaggcacactctttccctacacgacgctcttccgatct

SEQ ID NO: 19 - DNA, 70 bp, Artificial Sequence
  D503
aatgatacggcgaccaccgagatctacaccctatcctacactctttccctacacgacgctcttccgatct

SEQ ID NO: 20 - DNA, 70 bp, Artificial Sequence
  D504
aatgatacggcgaccaccgagatctacacggctctgaacactctttccctacacgacgctcttccgatct

SEQ ID NO: 21 - DNA, 70 bp, Artificial Sequence
  D505
aatgatacggcgaccaccgagatctacacaggcgaagacactctttccctacacgacgctcttccgatct

SEQ ID NO: 22 - DNA, 70 bp, Artificial Sequence
  D506
aatgatacggcgaccaccgagatctacactaatcttaacactctttccctacacgacgctcttccgatct

SEQ ID NO: 23 - DNA, 70 bp, Artificial Sequence
  D507
aatgatacggcgaccaccgagatctacaccaggacgtacactctttccctacacgacgctcttccgatct

SEQ ID NO: 24 - DNA, 70 bp, Artificial Sequence
  D508
aatgatacggcgaccaccgagatctacacgtactgacacactctttccctacacgacgctcttccgatct

SEQ ID NO: 25 - DNA, 65 bp, Artificial Sequence
  D701
caagcagaagacggcatacgagatcgagtaatgtgactggagttcagacgtgtgctcttccgatc

SEQ ID NO: 26 - DNA, 65 bp, Artificial Sequence
  D702
caagcagaagacggcatacgagattctccggagtgactggagttcagacgtgtgctcttccgatc

SEQ ID NO: 27 - DNA, 65 bp, Artificial Sequence
  D703
caagcagaagacggcatacgagataatgagcggtgactggagttcagacgtgtgctcttccgatc

SEQ ID NO: 28 - DNA, 65 bp, Artificial Sequence
  D704
caagcagaagacggcatacgagatggaatctcgtgactggagttcagacgtgtgctcttccgatc

SEQ ID NO: 29 - DNA, 65 bp, Artificial Sequence
  D705
caagcagaagacggcatacgagatttctgaatgtgactggagttcagacgtgtgctcttccgatc

SEQ ID NO: 30 - DNA, 65 bp, Artificial Sequence
  D706
caagcagaagacggcatacgagatacgaattcgtgactggagttcagacgtgtgctcttccgatc

SEQ ID NO: 31 - DNA, 65 bp, Artificial Sequence
  D707
caagcagaagacggcatacgagatagcttcaggtgactggagttcagacgtgtgctcttccgatc

SEQ ID NO: 32 - DNA, 65 bp, Artificial Sequence
  D708
caagcagaagacggcatacgagatgcgcattagtgactggagttcagacgtgtgctcttccgatc

SEQ ID NO: 33 - DNA, 65 bp, Artificial Sequence
  D709
caagcagaagacggcatacgagatcatagccggtgactggagttcagacgtgtgctcttccgatc

SEQ ID NO: 34 - DNA, 65 bp, Artificial Sequence
  D710
caagcagaagacggcatacgagatttcgcggagtgactggagttcagacgtgtgctcttccgatc

SEQ ID NO: 35 - DNA, 65 bp, Artificial Sequence
  D711
caagcagaagacggcatacgagatgcgcgagagtgactggagttcagacgtgtgctcttccgatc

SEQ ID NO: 36 - DNA, 65 bp, Artificial Sequence
  D712
caagcagaagacggcatacgagatctatcgctgtgactggagttcagacgtgtgctcttccgatc

SEQ ID NO: 37 - DNA, 23 bp, Artificial Sequence
  T1 fragment
ggggccactagggacaggattgg

SEQ ID NO: 38 - DNA, 23 bp, Artificial Sequence
  T2 fragment
caggcgggctcacctccgtgtgg

SEQ ID NO: 39 - DNA, 40 bp, Artificial Sequence
  H1 fragment
cgagggctagaattacctaccggcctccaccatgcctgcg

SEQ ID NO: 40 - DNA, 300 bp, Artificial Sequence
  H3 fragment
gtacgggcaacccgagaaggctgagcctggactcaacgggttgctgggtggactccagactcggggcgacgactcttcacgcgcagagcaagggcgtcgagcggtcgtgaaagtcttagtaccgcacgtgccgactcactggggatattgcctggagctgtaccgttctaggggggggaggttggagacctcctcttctcacgactggacccgcgagggccgcgttgccggttcccccagaggctgaagaacaagggcttactgtgggcagggggacgcccattcagcggctggcgcttt

SEQ ID NO: 41 - DNA, 66 bp, Artificial Sequence
  pBPS_fwr
  misc_feature (34)..(41): n is a, c, g, or t
  misc_feature (42)..(47): multiplex_tag
acactctttccctacacgacgctcttccgatctnnnnnnnnnnnnnnttcggttagagcggatgtg

SEQ ID NO: 42 - DNA, 70 bp, Artificial Sequence
  pBPS_rev
  misc_feature (34)..(41): n is a, c, g, or t
  misc_feature (42)..(50): multiplex_tag
gtgactggagttcagacgtgtgctcttccgatcnnnnnnnnnnnnnnnnnaggtaacccatatgcatggc

SEQ ID NO: 43 - DNA, 61 bp, Artificial Sequence
  Hi primer
ttgccctctctcttcattcagggtcatgagaggcacgccattcaaggggagaagtgagatc

SEQ ID NO: 44 - DNA, 42 bp, Artificial Sequence
  H4 primer
aagaacttttctatttctgggtaggcatcatcaggagcagga

SEQ ID NO: 45 - DNA, 66 bp, Artificial Sequence; identical to SEQ ID NO: 41
  pBPS_fwr
  misc_feature (34)..(41): any amino acid
  misc_feature (42)..(48): multiplexing tag

SEQ ID NO: 46 - DNA, 70 bp, Artificial Sequence; identical to SEQ ID NO: 42
  pBPS_rev
  misc_feature (34)..(41): any amino acid
  misc_feature (42)..(50): multiplexing tag

SEQ ID NO: 47 - DNA, 300 bp, Artificial Sequence
  oligonucleotide pool
  misc_feature (29)..(272): 244 sequence randomly seleted from human genome assembly GRCh38
gcttattcgtgccgtgttatggcgcgccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcggccgcgggcacagcaatcaaaagta

SEQ ID NO: 48 - DNA, 20 bp, Artificial Sequence
  priming site
gcttattcgtgccgtgttat

SEQ ID NO: 49 - DNA, 20 bp, Artificial Sequence
  priming site
gggcacagcaatcaaaagta

(19) United States
(12) Patent Application Publication
(10) Pub. No.: US 2024/0336912 A1
(43) Pub. Date: Oct. 10, 2024

(54) IN VIVO DNA ASSEMBLY AND ANALYSIS

(71) Applicant: The Board of Trustees of the Leland Stanford Junior University, Stanford, CA (US)

(72) Inventors: Sasha Levy, Stanford, CA (US); Xianan LIU, Stanford, CA (US); Takeshi Matsui, Stanford, CA (US); Darach Miller, Stanford, CA (US); Weiyi LI, Stanford, CA (US)

(21) Appl. No.: 18/279,440

(22) PCT Filed: Mar. 4, 2022
(86) PCT No.: PCT/US2022/019012

§ 371 Date: Aug. 30, 2023

Related U.S. Application Data
(60) Provisional application No. 63/157,498, filed on Mar. 5, 2021.
(60) Provisional application No. 63/157,497, filed on Mar. 5, 2021.

(51) Int. Cl. C12N 15/10; C12N 15/11; C12N 15/66; C40B 50/06

(52) U.S. Cl. CPC C12N 15/1031; C12N 15/11; C12N 15/66; C40B 50/06

(57) ABSTRACT

Provided herein, inter alia, are methods and compositions for assembling oligonucleotide fragments in vivo. The methods bypass inefficient cloning methods and the requirement for expensive enzymes. The methods may further be used to assemble long fragments of DNA. The methods may be used to generate variant libraries and combinatorial libraries and may be used to trace biological processes. Also provided herein, inter alia, are methods for in vivo DNA barcoding of oligonucleotide sequences. Methods provided herein are contemplated to produce unique barcode-oligonucleotide fusion sequences for identifying and isolating the oligonucleotide sequences, e.g., from a mixture. Accordingly, also provided are methods of identifying an oligonucleotide from a mixture of oligonucleotides.

### RELATED APPLICATIONS

This application claims the benefit of priority under 35 U.S.C. § 119(e) to U.S. Provisional Application No. 63/157,497, filed Mar. 5, 2021, and U.S. Provisional Application No. 63/157,498, filed Mar. 5, 2021, the entire contents of each of which is incorporated herein by reference in their entireties.

### STATEMENT AS TO RIGHTS TO INVENTIONS MADE UNDER FEDERALLY SPONSORED RESEARCH AND DEVELOPMENT

This invention was made with government support under DOE FWP #100582 awarded by the Department of Energy, and NIST IAA P18-630-0001 awarded by the National Institute for Standards and Technology. The government has certain rights in the invention.

### INCORPORATION BY REFERENCE OF SEQUENCE LISTING

The contents of the sequence listing text file named “41243-570001WO_Sequence_Listing_ST25.txt”, which was created on Feb. 15, 2022 and is 24,576 bytes in size, is hereby incorporated by reference in its entirety.

### BACKGROUND

Recent advances in recombinant oligonucleotide technology have ignited research in the fields of traditional biology and bioengineering. However, oligonucleotide assembly processes can be expensive and time-consuming, with requirements for multiple purification steps and various enzymes. Moreover, current molecular biology methods have limitations in the size and composition of DNA elements that can be combined. Thus, methods for assembling DNA elements (e.g. promoters, gene fragments, etc.) together are needed to address these limitations and bypass the necessity for numerous and expensive enzymes (e.g. ligases, etc.). New methods are required for efficient, high throughput, and versatile assembly of DNA fragments on a massively parallel scale.

Advances in sequencing technologies allow for identification of long fragments of DNA. However, identifying and isolating unique DNA sequences in complex mixtures remains challenging due to complex purification requirements, low sample recovery or inefficient sequencing workflows, among other problems.

Provided herein, inter alia, are solutions to these and other problems in the art.

### BRIEF SUMMARY OF THE INVENTION

Provided herein, inter alia, are methods and compositions for in vivo assembling of DNA elements and DNA barcoding of oligonucleotide sequences.

The invention provides methods of assembling a plurality of DNA elements into an assembled DNA element within a recipient cell, the method comprising: (a) contacting a first donor cell comprising a first donor plasmid with a recipient cell comprising a recipient oligonucleotide under conditions to (i) transfer the first donor plasmid from the first donor cell to the recipient cell by conjugation and (ii) recombine the first donor plasmid and the recipient oligonucleotide in the recipient cell by homologous recombination wherein the first donor plasmid comprises, in sequential order, an optional first endonuclease site (C1), a first homologous recombination region (HR1), a first oligonucleotide comprising a first DNA element fragment (oligo1), a second homologous recombination region (HR2) comprising two homologous recombination regions (HR2.1, HR2.2) and an optional third endonuclease site (C3); the recipient oligonucleotide comprises a third homologous recombination region (HR3) homologous to HR1 and a fourth homologous recombination region (HR4) homologous to HR2.2; thereby providing, following the homologous recombination of HR1 with HR3 and HR2.2 with HR4, a first recombined recipient oligonucleotide comprising the first DNA element fragment; (b) contacting a second donor cell comprising a second donor plasmid with the recipient cell comprising the first recombined recipient oligonucleotide under conditions to (i) transfer the second donor plasmid from the second donor cell to the first recipient cell by conjugation and (ii) recombine the second donor plasmid and the first recombined recipient oligonucleotide to form a second recombined recipient oligonucleotide in the recipient cell by homologous recombination; wherein the second donor plasmid comprises, in sequential order, an optional fifth endonuclease site (C5), a fifth homologous recombination region (HR5) homologous to HR2.1, a second oligonucleotide encoding a second DNA element fragment (oligo2), a sixth homologous recombination region (HR6) comprising two homologous recombination regions (HR6.1, HR6.2), and an optional sixth endonuclease site (C6); thereby providing, following the homologous recombination of HR5 with HR2.1 and HR6.2 with HR4, a second recombined recipient oligonucleotide comprising the first and second DNA element fragments (oligo1, oligo2), which form a DNA assembly. In embodiments, HR2.1 and HR2.2 flank a non-homologous region comprising one (C2) or two endonuclease sites (C2.1, C2.2);
optionally wherein HR3 and HR4 flank a non-homologous region comprising one (C4) or two endonuclease sites (C4.1, C4.2). In embodiments, HR6.1 and HR6.2 flank a non-homologous region comprising one (C7) or two endonuclease sites (C7.1, C7.2). In embodiments, the recipient oligonucleotide is in a recipient cell plasmid or the recipient cell genome. In embodiments, the DNA assembly comprises at least a portion of a gene, a promoter, an enhancer, a terminator, an intron, an intergenic region, a barcode, a guide RNA (gRNA), or a combination thereof.

In embodiments, step (b) is repeated for one or more iterations with a third or subsequent donor cell comprising a third or subsequent donor plasmid comprising compatible HR regions and a third or subsequent oligonucleotide encoding a third or subsequent DNA element fragment (oligo3, oligo4, . . . oligoN), thereby forming a third or subsequent recombined recipient oligonucleotide comprising the first, the second, and a third or subsequent DNA element fragments which together form a DNA assembly.

In embodiments, step (a) comprises a plurality of first donor cells, each comprising a different first donor plasmid; and step (b) comprises a plurality of second, third, or subsequent donor cells, each comprising a different second, third, or subsequent donor plasmid; optionally wherein each first donor cell is in a position in a first ordered array and each second, third, or subsequent donor cell is in a position in a second, third, or subsequent ordered array; optionally wherein the method generates a combinatorial library comprising a plurality of different assembled DNA elements.

In embodiments, an oligonucleotide encoding a first endonuclease targeting the first, third, and/or fourth endonuclease site is present on the first donor plasmid and/or is present in the recipient cell. In embodiments, an oligonucleotide encoding a second endonuclease targeting the second, fifth, and/or sixth endonuclease site is present on the second donor plasmid and/or is present in the recipient cell. In embodiments, expression of the first and/or the second endonuclease is inducible and the method further comprises inducing expression of the first and/or second endonuclease. In embodiments, the first and/or the second endonuclease is selected from an RNA-guided endonuclease, a homing endonuclease, a transcription activator-like effector nuclease, and a zinc finger nuclease.

In embodiments, the first, second or subsequent donor plasmid comprises a selectable marker selecting for integration of the first oligonucleotide, second oligonucleotide or subsequent oligonucleotide into the recipient oligonucleotide; optionally wherein the selectable marker is within a non-homologous region between HR2.1 and HR2.2 and/or between HR6.1 and HR6.2, and/or between subsequent HR regions. In embodiments, the recipient oligonucleotide comprises a counter-selectable marker selecting against recipient cells that do not comprise the first, second, third, or subsequent oligonucleotide; optionally wherein the counter selectable marker is within a non-homologous region between HR2.1 and HR2.2 and/or between HR6.1 and HR6.2, and/or between subsequent HR regions.

In embodiments, the donor plasmid comprises an origin of transfer.

In embodiments, the donor plasmid comprises a conditional replication origin. In embodiments, the conditional replication origin is dependent on presence of an oligonucleotide or on a condition of cell growth. In embodiments, the donor plasmid or recipient oligonucleotide comprises an inducible high-copy replication origin.

In embodiments, the donor plasmid or recipient oligonucleotide comprises a replicon that can replicate plasmids of lengths greater than 30 kilobases.

In embodiments, the donor plasmid or recipient oligonucleotide is a yeast artificial chromosome (YAC), a mammalian artificial chromosome (MAC), a human artificial chromosome (HAC), or a plant artificial chromosome.

In embodiments, the donor plasmid or recipient oligonucleotide is a viral vector.

In embodiments, the donor plasmid comprises an oligonucleotide that enables plasmid conjugation.

In embodiments, the donor plasmid or the recipient cell comprises an oligonucleotide encoding one or more homologous DNA repair genes; optionally wherein expression of the one or more homologous DNA repair genes is inducible.

In embodiments, the donor plasmid or recipient cell comprises an oligonucleotide encoding one or more recombination-mediated genetic engineering genes.

In embodiments, the donor cell and the recipient cell are independently a bacteria cell; optionally wherein the bacteria cell is E. coli, Vibrio natriegens or V. cholerae.

In embodiments, the assembled DNA element is from 100 nucleotides to 500,000 nucleotides in length.

In embodiments, the first, second or subsequent homologous recombination (HR) regions and their corresponding HR regions on the recipient oligonucleotide each comprise from about 20 base pairs to about 500 base pairs; optionally about 50 to 100 base pairs.

In embodiments, any of the foregoing methods may further comprise one or more steps of lysing the recipient cells; amplifying an assembled DNA element; isolating an assembled DNA element; isolating a recipient oligonucleotide; sequencing an assembled DNA element; and sequencing a recipient oligonucleotide.

In embodiments, the steps of contacting the first and second or subsequent donor cells with the first recipient cell are performed simultaneously; optionally wherein only a final donor plasmid comprises a selectable marker or each donor plasmid comprises a selectable marker not present on the recipient oligonucleotide.

In an embodiment of any one of the foregoing methods, the donor plasmid comprising the last DNA element to form part of an assembled DNA element comprises a barcode homologous recombination (BHR) region to produce recipient cells each containing a recombined recipient oligonucleotide comprising the assembled DNA element, the BHR, and a further HR; and the method further comprises (i) constructing or acquiring an array of barcode donor cells, each containing a barcode donor plasmid comprising an HR homologous to the BHR, a unique barcode oligonucleotide, and a second HR homologous to the further HR of the recombined recipient oligonucleotide; (ii) contacting the array of barcode donor cells with an array of the recipient cells under conditions to (a) transfer the barcode donor plasmids from the barcode donor cells to the recipient cells by conjugation and (b) recombine the barcode donor plasmids and recipient oligonucleotides in the recipient cells by homologous recombination, thereby producing an array of recipient cells comprising barcoded assemblies.

In embodiments of any one of the foregoing methods, each donor plasmid comprises a further pair of unique endonuclease sites CX, CY, flanking a barcode homologous recombination (BHR) region and the method further comprises contacting an array of recipient cells, each comprising a DNA assembly, with an array of barcode donor cells, each containing a barcode donor plasmid comprising a pair of HR regions homologous to the BHR flanking a unique barcode oligonucleotide, to produce an array of recipient cells comprising barcoded assemblies.

In embodiments of any one of the foregoing methods, the method further comprises contacting a reset donor cell comprising a reset donor plasmid with a recipient cell comprising a recombined recipient oligonucleotide, wherein the reset donor plasmid comprises, in sequential order, a homologous recombination region (HRt) homologous to a terminal sequence of the DNA assembly, a reset endonuclease site, a selectable marker, a reset endonuclease site, a homologous recombination region (HRX), and an origin of transfer; wherein the recombined recipient oligonucleotide comprises, in sequential order, a reset endonuclease site, the DNA assembly, a homologous recombination region homologous to HRX (HRXa) and a reset endonuclease site; thereby providing, subsequent to homologous recombination between the HRt and the terminal sequence of the DNA assembly and between the HRX and the HRXa, a reset plasmid comprising the origin of transfer and the DNA assembly. In embodiments, the reset plasmid is in a donor cell. In embodiments, the reset plasmid contains a restricted origin of replication that functions in both donor cells and recipient cells. In embodiments, the reset donor plasmid is constructed by a method comprising introducing an oligonucleotide insert comprising homologous recombination regions HRt, HRX, flanking two endonuclease sites (C1, C2) and a counter-selectable marker (CM), HRt-C1-CM-C2-HRX; or a library of such oligonucleotide inserts; allowing an endonuclease to cleave the endonuclease sites and introducing a counter-selectable marker at the cleavage sites using homologous recombination.

In embodiments of any of the foregoing methods, the recipient oligonucleotide comprises a mobile genetic element capable of transferring a DNA assembly to other cell types including yeast cells, plant cells, mammalian cells, or other bacterial cells.

In embodiments of any of the foregoing methods, the method comprises utilizing two or more recipient oligonucleotides having compatible homologous recombination regions to construct a DNA library.

In embodiments of any of the foregoing methods, the oligonucleotide of the donor plasmid comprises a first linker oligonucleotide homologous to a terminal sequence of a first DNA assembly and a second linker oligonucleotide homologous to a second oligonucleotide. In embodiments, the linker oligonucleotide further comprises an additional DNA element fragment that is not homologous to the first DNA assembly or second DNA oligonucleotide. In embodiments, the method is used to assemble a mutagenesis library; to combine genetic regions such as genes, promoters, terminators, and regulatory regions from different species; to construct and/or combine genetic regulatory pathways; to construct combinatorial gRNA libraries; or to assemble arrays of bacteria containing plasmids for screening assays.

In embodiments of any of the foregoing methods, prior to steps (a) and (b), the first and second oligonucleotides comprising the first and second DNA element fragments are inserted into the first and second donor plasmids.

The invention also provides methods of conjugating barcodes to oligonucleotides, the methods comprising (a) inserting each oligonucleotide of a mixture of oligonucleotides into a donor plasmid, each donor plasmid comprising, in sequential order, optionally a first endonuclease site (C1), a first homologous recombination region (HR1), a second homologous recombination region (HR2), and optionally a second endonuclease site (C2); wherein each oligonucleotide is inserted between HR1 and HR2, thereby providing a plurality of donor plasmids comprising donor oligonucleotides, each donor plasmid comprising a single donor oligonucleotide from the mixture of oligonucleotides: C1-HR1-oligo-HR2-C2; (b) transforming a plurality of cells with the plurality of donor plasmids such that each cell comprises a donor plasmid, thereby forming a plurality of donor cells; (c) plating and culturing the plurality of donor cells, each in a unique position on a first ordered array, thereby providing a first ordered array of donor cells; (d) providing a plurality of recipient cells in a second ordered array, wherein each recipient cell comprises a recipient oligonucleotide comprising, in sequential order, a unique barcode sequence, wherein the unique barcode sequence identifies a position of the recipient cell in the second ordered array, a third homologous recombination region (HR3) homologous to HR1, optionally a third endonuclease site (C3), and a fourth homologous recombination region (HR4) homologous to HR2; (e) contacting the first ordered array of donor cells with the second ordered array of recipient cells under conditions to (i) transfer the donor plasmids from the donor cells to the recipient cells in corresponding positions on the array by conjugation, (ii) optionally cleave the first, second, and third endonuclease sites, and (ii) transfer the oligonucleotides from the donor plasmids to the recipient cell oligonucleotides by homologous recombination, thereby forming an third array of fusion oligonucleotides, each comprising a unique barcode sequence and a donor oligonucleotide from the mixture of oligonucleotides; and (f) optionally sequencing the fusion oligonucleotides, and thereby identifying each oligonucleotide in the array of by its barcode sequence. In embodiments, the recipient oligonucleotide is in a recipient cell plasmid or the recipient cell genome. In embodiments, the donor plasmid comprises a selectable marker between HR1 and HR2 selecting for integration of the oligonucleotide into the recipient cell oligonucleotide; optionally wherein the donor plasmid comprises a counter-selectable marker. In embodiments, the recipient cell oligonucleotide comprises a fourth endonuclease site (C4).

The invention also provides methods of identifying an oligonucleotide from a plurality of oligonucleotides, the methods comprising (a) providing a plurality of donor cells in a first ordered array, wherein each donor cell comprises a donor plasmid, each donor plasmid comprising, in sequential order, optionally a first endonuclease site (C1), a first homologous recombination region (HR1), a unique barcode sequence, a second homologous recombination region (HR2), and optionally a second endonuclease site (C2), wherein the unique barcode sequence identifies a position of the host cell in the first ordered array; (b) providing a plurality of recipient cells, wherein each recipient cell comprises a recipient plasmid comprising, in sequential order, an oligonucleotide from the plurality of oligonucleotides, a third homologous recombination region (HR3) homologous to HR1, optionally a third endonuclease site (C3), and a fourth homologous recombination region (HR4) homologous to HR2; (c) plating and culturing the plurality of recipient cells, each in a unique position on a second ordered array, thereby providing a second ordered array of recipient cells; (d) contacting the first ordered array with the second ordered array under conditions to (i) transfer the donor plasmids from the donor cells to the recipient cells in corresponding positions on the array by bacterial conjugation, (ii) cleave the first, second, and third endonuclease sites, and (ii) transfer the barcode sequences from the donor plasmids to the recipient cell oligonucleotides by homologous recombination, thereby forming an third array of fusion oligonucleotides, each comprising a unique barcode sequence and an oligonucleotide from the mixture of oligonucleotides; and (e) sequencing the fusion oligonucleotides, thereby identifying each oligonucleotide in the array by its barcode sequence. In embodiments, the recipient oligonucleotide is in a recipient cell plasmid or the recipient cell genome. In embodiments, the donor plasmid comprises a selectable marker between HR1 and HR2 selecting for integration of the barcode sequence into the recipient cell oligonucleotide; optionally wherein the donor plasmid comprises a counter-selectable marker. In embodiments, the recipient cell oligonucleotide comprises a fourth endonuclease site (C4). In embodiments, the first endonuclease site, the second endonuclease site, and the third endonuclease site are the same or different. In embodiments, the donor plasmid comprises an origin of transfer, and/or a conditional replication origin; optionally wherein the origin of transfer is from a mobile element; further optionally wherein the conditional replication origin depends on the presence of an oligonucleotide or a condition of cell growth. In embodiments, the donor plasmid or recipient plasmid comprises a replicon that can replicate plasmids at least 30 kilobases in length, optionally wherein the replicon is from a P1-derived artificial chromosome or a bacterial artificial chromosome. In embodiments, the donor plasmid or recipient cell oligonucleotide comprises an inducible high-copy replication of origin. In embodiments, the donor plasmid or recipient cell oligonucleotide comprises a yeast artificial chromosome (YAC), a mammalian artificial chromosome (MAC), a human artificial chromosome (HAC), or a plant artificial chromosome. In embodiments, the donor plasmid or recipient cell oligonucleotide comprises a viral vector. In embodiments, the endonuclease sites are cleaved by one or more endonucleases encoded by one or more oligonucleotides in the recipient cell and/or encoded in the donor plasmid; optionally wherein the one or more endonucleases is a homing endonuclease or an RNA-guided DNA endonuclease; further optionally wherein the endonuclease is HO. In embodiments, the donor cell or recipient cell comprises an oligonucleotide that (i) enables plasmid conjugation; (ii) encodes one or more homologous DNA repair genes; or (iii) encodes one or more recombination-mediated genetic engineering genes. In embodiments, the donor cells, recipient cells, or recombinant recipient cells are transferred to positions on a third ordered array, a fourth ordered array, or a subsequent ordered array. In embodiments, the donor cell and the recipient cells are independently bacteria cells optionally wherein the bacteria cell is E. coli, Vibrio natriegens or V. cholerae. In embodiments, the barcode sequence is from about four nucleotides to about 100 nucleotides in length, optionally wherein the barcode sequence is about 30 nucleotides in length. In embodiments, the mixture of oligonucleotides is a product of a DNA synthesis or assembly technology selected from chemical coupling, template-independent enzymatic synthesis using polymerase nucleotide conjugates, polymerase chain assembly (polymerase cycling assembly), Gibson assembly (Chew back, anneal and repair), ligase chain reaction/ligase cycling reaction, Phi29 polymerase, rolling circle, loop mediated isothermal (LAMP), strand displacement (SDA), helicase dependent (HAD), recombinase polymerase (RPA), nucleic acid sequences based amplification (NASBA), Golden Gate cloning, MoClo Cloning, BioBricks or assembled BioBricks, thermodynamically balanced inside out synthesis, DNA cloning, ligation-independent cloning, ligation by selection cloning, recombineering, yeast assembly, PCR, capture by molecular inversion probes or LASSO probes, DropSynth, and enzymatic DNA synthesis. In embodiments, the mixture of oligonucleotides is a product of a pooled mutagenesis technology selected from a polymerase chain reaction technology including error-prone PCR, PCR with degenerate oligos, and regular PCR, chemical or light mutagenesis, in vitro synthesis with library of editing oligos, in vivo editing, for example MAGE, MAGESTIC, CRISPR, prime editing, retron editing, and base modification with CRISPR, TALENs and zing finger nucleases. In embodiments, the mixture of oligonucleotides comprises at least a fragment of genomic DNA, cDNA, organelle DNA, or natural plasmid DNA. In embodiments, the mixture of oligonucleotides comprises captured or amplified DNA originating from gDNA, cDNA or organelle DNA, for example from a balanced cDNA library, a PCR product such as a multiplex PCR product, molecular inversion probes, including LASSO probes, capture by annealing or subtractive hybridization, co-transformation and homologous recombination, rolling circle amplification, or LAMP. In embodiments, the mixture of oligonucleotides comprises captured or amplified DNA from a plasmid or plasmid library, for example an open reading frame (ORF) library, a promoter library, a terminator library, an intron library, a BAC library, a PAC library, a lentiviral library, a gRNA library, PCR products, restriction digestion products, or GATEWAY shuttling products. In embodiments, the oligonucleotides of the mixture of oligonucleotides are integrated into a donor plasmid by a method comprising co-transformation and recombineering; transformation and recombineering; or conjugation and recombineering. In embodiments, the method of co-transformation and recombineering comprises constructing a linear or circular donor plasmid comprising a selectable marker and two homologous recombination regions which are each homologous to sequences at the termini of the oligonucleotides in the mixture; co-transforming the donor plasmid and oligonucleotides into cells; inducing homologous recombination; and selecting for the selectable marker; optionally wherein the method is performed with a library or pool of donor plasmids and/or oligonucleotides. In embodiments, the method of transformation and recombineering comprises constructing linear or circular donor plasmids comprising a selectable marker and two homologous recombination regions which are each homologous to sequences at the termini of the oligonucleotides in the mixture, wherein the oligonucleotides reside on plasmids within host cells; transforming the host cells with the donor plasmids; inducing homologous recombination; and selecting for the selectable marker. In embodiments, the method of conjugation and recombineering comprises constructing linear or circular donor plasmids comprising a counter-selectable marker (−1) flanked by two optional endonuclease sites and two homologous recombination (HR) regions; wherein the donor plasmids reside in donor cells containing a crippled F-plasmid, which can induce conjugation but can not conjugate, and the oligonucleotides of the mixture reside on plasmids within recipient cells, each flanked by HR regions homologous to the HR regions of the donor plasmids and adjacent to at least one selectable marker (+1) to select for recombination of each oligonucleotide into a donor plasmid; providing a homologous recombinase and optionally one or more endonucleases, either in the recipient cells or encoded by the donor plasmids; contacting the donor cells and the recipient cells under conditions to (i) transfer the donor plasmids from the donor cells to the recipient cells by bacterial conjugation and (ii) recombine the donor plasmids and the recipient plasmids by homologous recombination; and selecting for cells comprising the selectable marker but not the counter-selectable marker. In embodiments, the method is performed with a library of donor and/or recipient plasmids. In embodiments, the oligonucleotides comprise a library such as an ORF library, a promoter library, a terminator library, an intron library, a BAC library, a PAC library, a lentiviral library, a gRNA library, a gDNA library, a cDNA library, a protein domain library, a promoter library, a terminator library, a library of regulatory elements, a library of structural elements, or a library of DNA variants derived from DNA mutagenesis. In embodiments, the mixture of oligonucleotides comprises arrays of cells comprising plasmid libraries, for example a gRNA library, a gDNA library, a cDNA library, an open reading frame (ORF) library, a protein domain library, a promoter library, a terminator library, a library of regulatory elements, a library of structural elements, or a library of DNA variants derived from DNA mutagenesis. In embodiments, the mixture of oligonucleotides comprises arrays of cells comprising DNA element fragments for use in the method of any one of claims 1-35.

### BRIEF DESCRIPTION OF THE DRAWINGS

FIG. 1. Schematic of an embodiment of a method for DNA assembly described herein showing donor plasmid and recipient oligonucleotide elements as shaded boxes. The figure shows three “rounds” of “DNA stitching” in each of which a new oligonucleotide comprising a DNA element fragment is added to the recipient oligonucleotide. Throughout the figures, DNA element fragments may be depicted variously as Input DNA 1, Input DNA 2, etc. before recombination and DNA1, DNA2, etc. following recombination; or alternatively as oligo1, oligo2, etc., or may be referred to more generally as “DNA blocks”. Boxes designated C1, C2, etc. refer to endonuclease sites; boxes designated HR1, HR2, etc. refer to homologous recombination regions; boxes designated oligo1, oligo2, etc. refer to an oligonucleotide comprising a DNA element fragment; boxes designated with a number and a plus or minus sign refer to selectable (+) and counter-selectable (−) markers. Not all elements shown in the schematic are required in every embodiment of the methods described here; for example, the markers and endonuclease sites designated C2.1, C2.2, C7.1, C7.2, are optional.

FIG. 2A-B. Panel A is a schematic map of an exemplary recipient oligonucleotide (in the form of a plasmid) used in the methods described herein. Panel B shows two schematic maps of exemplary donor plasmids and helper plasmids.

FIG. 3. CRISPR/Cas9 enhances DNA assembly, also referred to herein as “stitching”, efficiency. The number of colonies per 6×10⁶ cells on the selection plates. Two donor plasmids, only one of which expresses a functional gRNA, were transformed into each of three recipient strains: (1) BW28705 (no λ-red and no Cas9), (2) BW28705/pML300 (λ-red but no Cas9), (3) BW28705/pSL359 (Cas9 and λ-red).

FIG. 4. Schematic maps of exemplary plasmids that may be used in the in vivo DNA assembly or “stitching” methods described herein. In donor cells, a conjugation-competent helper plasmid may contain the genes for plasmid transfer (Tra operon). To immobilize the conjugation plasmid itself, the origin of transfer (oriT) is replaced with a selectable marker (+6). A donor plasmid contains a swapping cassette (+1/−1 or +2/−2), two homology regions (H2 and H3), four endonuclease cut sites (two circles labeled with 1, and two circles labeled with 2), a backbone selectable marker (+4), a conditional replication origin (R6K) that depends on an allele in donor's genome (pir1-116), the oriT sequence, and a gRNA expression cassette (gRNA1 or gRNA2).

FIG. 5. Schematic maps of exemplary plasmids that may be used in in vivo stitching methods described herein. In recipient cells, the helper plasmid contains a rhamnose-inducible red operon (P_{rhaBAD}-red), an arabinose-inducible Cas9 (P_{araBAD}-Cas9), an E. coli RecA gene for boosting homologous recombination, a backbone selectable marker (+5), and a curable origin of replication (pSC101 ori^{TS}). The recipient plasmid includes two endonuclease cut sites (two circles labeled with 1), a swapping cassette (+2/−2), two homology regions (H2 and H3), and a replication origin (ColE1). +1: HygR; +2:NsrR; −1: SacB; −2: PheS; +3: GmR; +4: KanR; +5: SpR; +6: TcR.

FIG. 6. Schematic overview of an exemplary method of in vivo stitching. Donor plasmids carrying a DNA fragment (upward or downward striped rectangles) are introduced into donor plasmids and donor cells. Donor plasmids are conjugated to recipient cells and a DNA fragment is transferred from the donor plasmid to the recipient plasmid. Plasmids are cut using CRISPR/Cas9, which is induced by arabinose. A guide RNA on the donor plasmid (gRNA1 or gRNA2, alternating between assembly rounds) specifies a recognition sequence for cutting (“1” and “2” circles, alternating between assembly rounds). Homology regions on both the synthesized oligos and plasmid backbones (H1 and H3 in round 1) promote recombination, which is induced by rhamnose, and seamlessly stitch oligos together for gene assembly. Alternating selectable (+1 and +2) and counter-selectable (−1 and −2) markers on donor plasmids allow for recursive DNA transfers with a maximum gene length theoretically set by the maximum tolerable plasmid size. R6K and ColE1 are origins of replication. +3 and +4 are selectable markers used for plasmid maintenance.

FIGS. 7A-I. Examples of DNA assembly. Panel A shows three donor plasmids, each carrying a portion of mEGFP, that were sequentially conjugated and assembled into a recipient plasmid (3 stitches). Panel B shows fluorescence of colonies from a negative control, a positive control, and the in vivo stitching products after three rounds of assembly in liquid. Colonies represent independent conjugation and recombination events and 100% are fluorescent. Panel C shows arrayed assemblies of mEGFP in 96- and 384-position formats. All colonies appear fluorescent. Panel D shows the percentage of fluorescent colonies after a final round of liquid assembly using a third mEGFP fragment with different lengths of homology to the second mEGFP fragment. Panel E shows representative restriction digests of colonies containing various plasmids scraped from agar during an assembly. Expected product from the non-recombinant recipient plasmid cannot be observed after selection for recombinants or curing of the helper plasmid (arrow points). Panel F shows a schematic of the analysis of results from Sanger sequencing of 96 colonies following assembly of mEGFP. Sequencing products are derived from a colony PCR of a pipette tip touched to each colony. One colony contained a mid-product (the first round assembly product) and one contained a stitching error (a large deletion). Panel G shows the fluorescence of colonies from the in vivo stitching products after five rounds of assembly for 2 fluorescent genes, mPapaya and sfGFP, and 4 recombinase genes. Colonies may represent independent conjugation and recombination events. All mPapaya and sfGFP colonies are fluorescent. Panel H is the trace file from Sanger sequencing the mPapaya in vivo stitching products after five rounds of assembly. Alignment to the expected sequence shows that the assembly is 100% accurate and pure. Panel I shows results from an assembly of three ˜3 kb fragments, for a total assembly length of ˜9 kb. The recipient plasmids at various stages of assembly were digested with restriction enzymes to separate the stitching products from the vector backbone. The digested products were then subject to agarose gel electrophoresis to check the size of the stitching products (lanes 1-3). The gel bands corresponding to the stitching products are marked with an arrow. Linearized vector backbones without the stitching products are shown in lanes 5-6. Selectable and counter-selectable markers in the swapping cassette differ between assembly rounds, with the swapping cassette in the first and third assembly rounds being ˜1.5 kb longer than the swapping cassette in the original recipient plasmid or the second assembly round.

FIG. 8A-B. Schematics of exemplary donor and recipient plasmids at the beginning of the first round of DNA stitching. Panel A shows a schematic for both example plasmids, where the donor plasmid contains the first oligonucleotide (1). Panel B shows the legend of shapes used to illustrate the sequences corresponding to and example genome, positive selectable marker, negative selectable marker, origin of transfer (oriT), gRNA expression unit (gRNA), positional barcode, homology for recombination domain (H), inducible lambda red operon (λred), inducible I-SceI endonuclease, plasmid, inducible endonuclease (Cas9), gRNA target sites, I-SceI target sites, conjugation Tra operon, deleted oriT (oriA::TcR), temperature sensitive origin (pSC101 ori), conditional origin of replication (R6K), and recipient of origin of replication (ColE1). The same legend of shapes in FIG. 8B is used for FIGS. 9-35.

FIG. 9. Schematic of exemplary initial plasmids for use in the methods as described herein: donor plasmid containing the first oligo (1), recipient plasmid, and a plasmid containing oriT, which mediates the conjugation of the donor plasmid to the recipient cell.

FIG. 10. Schematic of a subsequent step in an exemplary method of DNA stitching using the donor and recipient plasmids depicted in FIG. 9: gRNA1 guides Cas9 in the recipient cells to generate site-specific double strand breaks on the donor and recipient plasmids (indicated by down-facing arrows).

FIG. 11. Schematic of a subsequent step in the exemplary method of DNA stitching depicted in FIG. 9-10: The dark shaded sequence elements here will be used as homology region for lambda Red mediated homologous recombination.

FIG. 12. Schematic of a subsequent step in the exemplary method of DNA stitching depicted in FIG. 9-11: Homologous recombination between the plasmids, showing where the sequence from the donor plasmid will be inserted into the recipient plasmid, and its orientation, with the assistance of a λ-red system.

FIG. 13. Schematic of a subsequent step in the exemplary method of DNA stitching depicted in FIG. 9-12: The fragment containing the first oligonucleotide is integrated into the recipient plasmid as shown.

FIG. 14. Schematic of a subsequent step in the exemplary method of DNA stitching depicted in FIG. 9-13: The plasmid will be selected for gaining the +2 positive selectable marker.

FIG. 15. Schematic of a subsequent step in the exemplary method of DNA stitching depicted in FIG. 9-14: The plasmid will be counter selected for loss of the previous counter selectable marker.

FIG. 16. Schematic of a subsequent step in the exemplary method of DNA stitching depicted in FIG. 9-15: The plasmid will additionally be selected for retaining the +3 positive selectable marker on the original recipient backbone.

FIG. 17. Schematic of a subsequent step in the exemplary method of DNA stitching depicted in FIG. 9-16: The second donor plasmid containing the second oligonucleotide is ready to be assembled into the previous ligation product (the new recipient plasmid) containing the first oligonucleotide.

FIG. 18. Schematic of a subsequent step in the exemplary method of DNA stitching depicted in FIG. 9-17: The oriT directs conjugation of the donor plasmid.

FIG. 19. Schematic of a subsequent step in the exemplary method of DNA stitching depicted in FIG. 9-18: A second gRNA expression guides Cas9 to generate double strand breaks at the sites indicated by the downward facing arrows.

FIG. 20. Schematic of a subsequent step in the exemplary method of DNA stitching depicted in FIG. 9-19: The highlighted regions (darker shaded regions) are the homology regions for recombination.

FIG. 21. Schematic of a subsequent step in the exemplary method of DNA stitching depicted in FIG. 9-20: The fragment containing the first oligonucleotide is integrated into the recipient plasmid as shown.

FIG. 22. Schematic of a subsequent step in the exemplary method of DNA stitching depicted in FIG. 9-21: The second oligonucleotide is assembled adjacent to the 3′-end of the first oligo in the recipient plasmid, generating a new recipient plasmid.

FIG. 23. Schematic of a subsequent step in the exemplary method of DNA stitching depicted in FIG. 9-22: The plasmid containing the first and second oligonucleotides will be selected for gaining of +1 positive selectable marker.

FIG. 24. Schematic of a subsequent step in the exemplary method of DNA stitching depicted in FIG. 9-23: The plasmid containing the first and second oligonucleotides will be selected for loss of −2 counter selectable marker.

FIG. 25. Schematic of a subsequent step in the exemplary method of DNA stitching depicted in FIG. 9-24: The plasmid containing the first and second oligonucleotides will also be selected for retaining the backbone selectable marker.

FIG. 26. Schematic of a subsequent step in the exemplary method of DNA stitching depicted in FIG. 9-25: The schematic for a donor plasmid containing oligonucleotide three and the recipient plasmid with oligonucleotides one and two to initiate the third round of DNA stitching.

FIG. 27. Schematic of a subsequent step in the exemplary method of DNA stitching depicted in FIG. 9-26: The oriT plasmid initiates conjugation.

FIG. 28. Schematic of a subsequent step in the exemplary method of DNA stitching depicted in FIG. 9-27: gRNA1 guides Cas9 in the recipient cells to generate site-specific double strand breaks on the donor and recipient plasmids (indicated by down-facing arrows).

FIG. 29. Schematic of a subsequent step in the exemplary method of DNA stitching depicted in FIG. 9-28: The highlighted sequences here will be used as homology region for lambda Red mediated homologous recombination.

FIG. 30. Schematic of a subsequent step in the exemplary method of DNA stitching depicted in FIG. 9-29: Post-homologous recombination, showing where and the orientation of the sequence from the donor plasmid will be inserted into the recipient plasmid.

FIG. 31. Schematic of a subsequent step in the exemplary method of DNA stitching depicted in FIG. 9-30: The third oligonucleotide is assembled adjacent to the 3′-end of the second oligonucleotide in the recipient plasmid, generating a new recipient plasmid.

FIG. 32. Schematic of a subsequent step in the exemplary method of DNA stitching depicted in FIG. 9-31: Analogous to round 1 of DNA stitching, the plasmid will be selected for gaining the +2 positive selectable marker.

FIG. 33. Schematic of a subsequent step in the exemplary method of DNA stitching depicted in FIG. 9-32: The plasmid will be counter selected for loss of the −1 counter selectable marker.

FIG. 34. Schematic of a subsequent step in the exemplary method of DNA stitching depicted in FIG. 9-33: The plasmid will be selected for retaining the backbone selectable marker.

FIG. 35A-B. Panel A diagrams a step in the methods exemplified in FIGS. 9-34 illustrating an embodiment in which subsequent oligonucleotides can be incorporated (up until the upper size limit for total sequence length is reached) in the same fashion, alternating between the two conjugation, double strand break processing, assembly, and selectable/counter selectable/backbone selection processes. Panel B is a schematic overview of an exemplary in vivo assembly in which two DNA element fragments (depicted in the figure as Input DNA 1, Input DNA 2 before recombination and DNA1, DNA2 following recombination; and which may also be referred to herein as oligo1, oligo2, etc., or more generally as “DNA blocks”) are added in a single round of conjugation. In each well, a recipient cell is conjugated first by a first donor cell comprising a first donor plasmid and second by a second donor cell comprising a second donor plasmid. A selectable marker introduced by the second donor plasmid and counter-selectable markers on the recipient plasmid and optionally on the first donor plasmid enable for selection of a recombinant assembly product containing oligonucleotides introduced by both donor plasmids.

FIG. 36A-D. Panel A is a schematic overview of an exemplary method for in vivo DNA analysis, as described herein. In this example, indexing barcodes are located on the recipient plasmid. Panel B is a schematic overview of another exemplary method for in vivo DNA analysis, as described herein. In this example, indexing barcodes are located on the donor plasmid and are added to an in vivo DNA assembly product using a homologous region at the end of the assembly product. In this example, the endonuclease sites used are the same as those used for in vivo DNA assembly. Panel C is a schematic overview of another exemplary method for in vivo DNA analysis, as described herein. In this example, indexing barcodes are located on the donor plasmid and are added to an in vivo DNA assembly product. In this example, endonuclease target sites (C) and homologous regions (boxes adjacent to C) are different from those used for in vivo DNA assembly. This example enables DNA analysis at multiple steps during an assembly. Panel D is a schematic overview of the method comprising a plasmid reset that moves a DNA assembly from a recipient plasmid to a donor plasmid to enable further rounds of assembly with larger DNA blocks. In part A of Panel D, a reset donor plasmid in a donor cell with homology to the beginning of the DNA assembly on the recipient plasmid is conjugated into the recipient cell. A site-specific endonuclease cleaves at “D” endonuclease target sites in both the reset donor plasmid and the recipient plasmid. Homologous recombination at regions adjacent to the “D” endonuclease target sites moves the DNA assembly cassette from the recipient plasmid to the reset donor plasmid. The reset donor plasmid is purified from the recipient cell and transformed into new donor cells where is can be utilized for further rounds of assembly. In part B of Panel D, a schematic shows a workflow for assembly of long DNA constructs. Small DNA blocks can be assembled into large DNA blocks using four rounds of DNA stitching. The large blocks are moved to donor plasmids and donor cells using reset donor plasmids. The large blocks can then be assembled into even larger blocks with further rounds of stitching.

FIG. 37 depicts schematic maps of exemplary plasmids for use in in vivo DNA analysis. In donor cells, a conjugation-competent helper plasmid contains the genes for plasmid transfer (Tra operon). To immobilize the helper plasmid itself, the origin of transfer (oriT) is replaced with a selectable marker (+6). The donor plasmid contains a swapping cassette (+ and −), two homology regions (H1 and H4), two sites for targeted plasmid cutting (ovals), a backbone selectable marker (+4), a conditionally replication origin (R6K) depending on an allele in donor's genome (pir1-116), and the oriT sequence. In recipient cells, the helper plasmid contains a lac-inducible red operon (P_{lac}-red), an E. coli RecA gene for boosting homologous recombination, a backbone selectable marker (+5), and a curable temperature-sensitive origin of replication (pSC101 ori^{TS}). The recipient plasmid includes two endonuclease cut sites (two ovals), a negative selectable marker (−3), and two homology regions (H1 and H4). Besides the two plasmids, recipient cells also have an integrated arabinose-inducible endonuclease I-SceI (P_{araBAD}-I-SceI) to generate DNA cleavage on target plasmids. +: HygR or NsrR; −: SacB or PheS; +3: GmR; −3: relE; +4: KanR; +5: SpR; +6: TcR.

FIG. 38 depicts schematic maps of exemplary plasmids for use in in vivo DNA analysis. Selectable markers: HygR, KanR, GmR, SpR. Counter-selectable markers: SacB, relE.

FIGS. 39A-B depicts schematic maps of an exemplary donor plasmid and recipient plasmid used for DNA parsing. Panel A shows the plasmid schematics in which the donor plasmid contains the first oligonucleotide. Panel B shows the legend of shapes used to illustrate the sequences corresponding to a genome, positive selectable marker, negative selectable marker, origin of transfer (oriT), gRNA expression unit (gRNA), positional barcode, homology for recombination domain (H), inducible lambda red operon (kred), inducible I-SceI endonuclease, plasmid, inducible endonuclease (Cas9), gRNA target sites, I-SceI target sites, conjugation Tra operon, deleted oriT (oriA::TcR), temperature sensitive origin (pSC101 ori), conditional origin of replication (R6K), and recipient of origin of replication (ColE1). The legend in Panel B also applies to FIGS. 40-46.

FIG. 40 shows a diagram of the donor and recipient plasmids for use in an example method as described herein.

FIG. 41 is an image that shows a second step in the example method of FIG. 40: gRNA1 guides Cas9 in the recipient cells to generate site-specific double strand breaks on the donor and recipient plasmids (indicated by down-facing arrows). SceI is I-SceI, a homing endonuclease.

FIG. 42 shows an image of a step in the example method of FIGS. 40 and 41: the H1 and H4 sequences here will be used as homology region for lambda Red mediated homologous recombination.

FIG. 43 is an image that shows a step in the example method of FIGS. 40-42: homologous recombination, showing where and the orientation of the sequence from the donor plasmid will be inserted into the recipient plasmid.

FIG. 44 is an image showing a step in the example method of FIGS. 40-43: the plasmid will be selected for gaining the + positive selectable marker.

FIG. 45 shows an image of a step in the example method of FIGS. 40-44: the plasmid will be counter-selected for loss of the previous counter-selectable marker.

FIG. 46 is an image that shows a step in the example method of FIGS. 40-45: the plasmid will additionally be selected for retaining the +3 positive selectable marker on the original recipient backbone.

FIGS. 47A-D. Panel A shows results from an experiment to determine the capability of in vivo DNA analysis to correctly identify the sequence at each position of a plate of arrayed donor cells containing a unique DNA barcode at each position. Each arrayed barcode donor was mated to two or three barcode recipient plates and recombinant cell colonies, each containing both a donor and recipient barcode, were selected on agar pads. Recombinant cells from plates were pooled and double barcodes were sequenced on an Illumina platform. Sequencing data was used to determine the percent of arrayed barcode donors that could be correctly indexed (recovery rate) when conjugated to one, two, or three separated barcode recipient arrays. No barcode donors were incorrectly assigned to the wrong position. Panel B shows results from an experiment to index and sequence verify a pool of 100 244-base oligonucleotides ordered from IDT as an oPool. The oligonucleotide pool was integrated into donor plasmids, which were then transformed into donor cells. Donor cells were randomly arrayed into 384-well plates at an expected frequency of less than one cell per well. Donor cells were conjugated to barcoded recipient cell arrays, and recombinant oligonucleotide-barcode recipient plasmids were sequenced using an Oxford Nanopore sequencer. Shown are the results of analysis of two 384-well plates. The shading indicates if the well had input DNA arrayed, if the sequence was a 100% match with one of the 244-base sequences in the oPool, and if the well was pure (i.e. only one 244-base sequence could be detected in the well). The positions marked as “100% matched pure well” would typically be used for downstream DNA assembly. Panel C is a histogram showing the distribution of errors between the consensus sequence determined by Oxford Nanopore sequencing and the expected DNA sequence in the oPool that is closest in sequence to the consensus, using data from the experiment in Panel B. Most wells contain an oligonucleotide that is identical to one of the sequences in the oPool. Panel D is a histogram showing the distribution of counts of independent clones recovered for each oligonucleotide that could be indexed, using data from the experiment in Panel B.

FIG. 48 is a schematic overview of a DNA assembly workflow to build directed combinatorial libraries from a set of input oligonucleotides. Pools of input DNA from multiple sources are integrated into donor plasmids and parsed into ordered arrays. Ordered arrays are re-arrayed to user-defined locations on multiple donor plates. Donor plates are sequentially conjugated to a recipient plate to assemble the desired constructs. An input oligonucleotide may be used in multiple assemblies by re-arraying donor cells containing that oligonucleotide to multiple positions on the donor plates.

FIG. 49 is a schematic overview of branching DNA assembly. A partial DNA assembly can be extended with multiple DNA blocks if homology regions are present. If homology regions are not present, a “DNA linker” must first be added to the partial DNA assembly. The DNA linker contains homology to the end of the partial DNA assembly and the beginning of the subsequent DNA block to be joined.

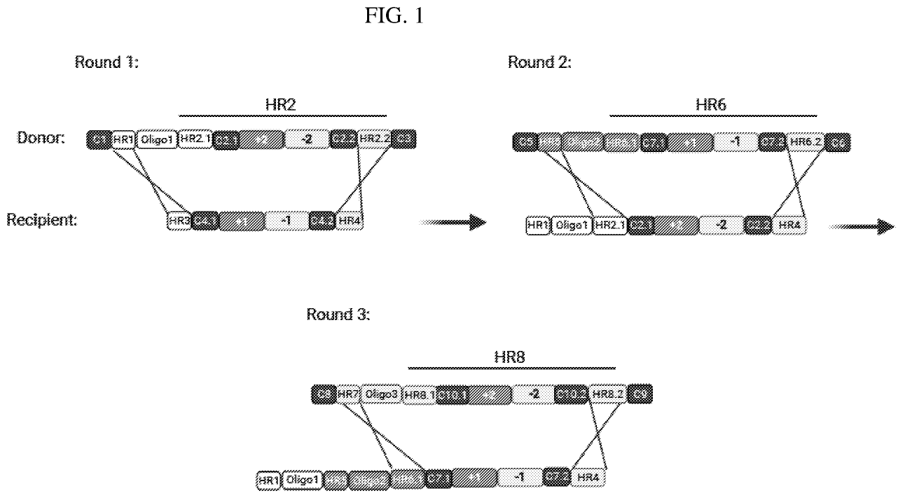

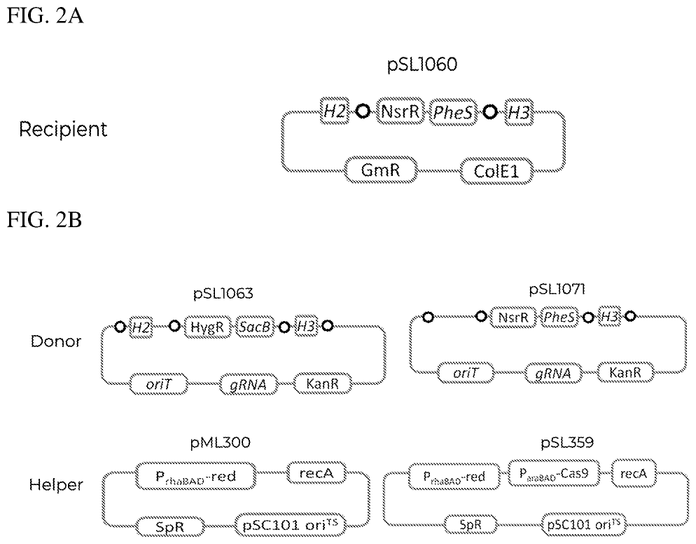

### DETAILED DESCRIPTION

### I. Definitions and Related Embodiments

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” means that the value may vary by +/−10%.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As may be used herein, the terms “nucleic acid,” “nucleic acid molecule,”” “nucleic acid sequence,” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, sgRNA, guide RNA, tracrRNA, a recombinant polynucleotide, a branched polynucleotide, a plasmid, a vector, isolated DNA of a sequence, isolated RNA of a sequence, a PCR product, a nucleic acid probe, and a primer. Polynucleotides useful in the methods of the disclosure may comprise natural nucleic acid sequences and variants thereof, artificial nucleic acid sequences, or a combination of such sequences.

“Nucleic acid” refers to nucleotides (e.g., deoxyribonucleotides or ribonucleotides) and polymers thereof in either single−, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids including known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids including one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

A “barcode” refers to one or more nucleotide sequences that are used to identify a cell or a plurality of cells with which the barcode is associated. Barcodes can be 3-1000 or more nucleotides in length, preferably 3-250 nucleotides in length, and more preferably 4-40 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. A barcode is “unique” when the barcode is (statistically) present in about one cell in a population of cells. The cell containing the barcode can then be expanded to make a clonal plurality of cells, such that each cell of the plurality of cells contains the same barcode. For example, “a plurality of barcoded cells, wherein each barcoded cell comprises a single, unique barcode” may refer to a population of cells which contains (statistically) a single cell containing a given barcode or a unique combination of barcodes. Alternatively, it may refer to a population of cells which contains a plurality of clonal populations of cells, each cell of each clonal population containing the same barcode, but cells of different clonal populations containing different barcodes.

As used herein, the term “complement,” refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

As used herein, the term “gene” is used in accordance with its plain ordinary meaning and refers to the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “expression vector” refers to a nucleic acid molecule that encodes for genes and/or regulatory elements necessary for the expression of genes. Expression of a gene from a vector, which may in the form of a plasmid, can occur in cis or in trans. If a gene is expressed in cis, the gene and regulatory elements are encoded by the same plasmid. Expression in trans refers to the instance where the gene and the regulatory elements are encoded by separate plasmids.

As used herein, the term “vector” refers to a nucleic acid molecule capable of transporting another nucleic acid to which it has been linked. A vector may be in the form of a “plasmid”, which in this context refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cell type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

In accordance with the methods described here, an oligonucleotide, plasmid or vector may contain at least one selectable marker. Selectable markers for use in the methods described herein may be any suitable selectable marker. In embodiments, and without limitation, the selectable marker is HygR, NsrR, ZeoR, TetA, CmR, SpR, GmR, mFabI, TmR, neoR, or kanR. In embodiments, the selectable marker is HygR. In embodiments, the selectable marker is NsrR. In embodiments, the selectable marker is ZeoR. In embodiments, the selectable marker is TetA. In embodiments, the selectable marker is CmR. In embodiments, the selectable marker is SpR. In embodiments, the selectable marker is GmR. In embodiments, the selectable marker is mFabI. In embodiments, the selectable marker is TmR. In embodiments, the selectable marker is neoR. In embodiments, the selectable marker is kanR.

In accordance with the methods described here, an oligonucleotide, plasmid or vector may contain at least one counter-selectable marker, e.g., one selecting for integration of the second or subsequent oligonucleotide into a recombined recipient oligonucleotide in the methods of assembling a DNA element described here. Counter-selectable markers for use in the methods described herein may be any suitable counter-selectable marker. In embodiments, and without limitation, the counter-selectable marker is PheS, SacB rpsL, tolC, galK, ccdB, tetA, thyA, lacY, gata-1, URA3, relE, mqsR, chpB, vhaV, or tse2. In embodiments, the counter-selectable marker is PheS. In embodiments, the counter-selectable marker is SacB. In embodiments, the counter-selectable marker is rpsL. In embodiments, the counter-selectable marker is tolC. In embodiments, the counter-selectable marker is galK. In embodiments, the counter-selectable marker is ccdB. In embodiments, the counter-selectable marker is ccdB. In embodiments, the counter-selectable marker is tetA. In embodiments, the counter-selectable marker is thyA. In embodiments, the counter-selectable marker is lacY. In embodiments, the counter-selectable marker is gata-1. In embodiments, the counter-selectable marker is URA3. In embodiments, the counter-selectable marker is relE. In embodiments, the counter-selectable marker is mqsR. In embodiments, the counter-selectable marker is chpB. In embodiments, the counter-selectable marker is vhaV. In embodiments, the counter-selectable marker is tse2.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, including the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be, e.g., about 100 to about 1000 base pairs in length.

A nucleotide base “position” is denoted by a number that sequentially identifies each amino acid (or nucleotide base) in the reference sequence based on its position relative to the 5′-end. Due to deletions, insertions, truncations, fusions, and the like that must be taken into account when determining an optimal alignment, in general the amino acid residue number in a test sequence determined by simply counting from the 5′-end will not necessarily be the same as the number of its corresponding position in the reference sequence. For example, in a case where a variant has a deletion relative to an aligned reference sequence, there will be no nucleotide base in the variant that corresponds to a position in the reference sequence at the site of deletion. Where there is an insertion in an aligned reference sequence, that insertion will not correspond to a numbered nucleotide position in the reference sequence. In the case of truncations or fusions there can be stretches of nucleotides in either the reference or aligned sequence that do not correspond to any nucleotide in the corresponding sequence.

The terms “numbered with reference to” or “corresponding to,” when used in the context of the numbering of a given polynucleotide sequence, refers to the numbering of the residues of a specified reference sequence when the given polynucleotide sequence is compared to the reference sequence.

As used herein, the term “virus” or “virus particle” is used according to its plain ordinary meaning within the context of viral transduction. Transduction with viral vectors can be used to insert or modify genes in mammalian cells.

As used herein, the terms “genetic modification”, “gene modification”, “gene editing”, “genetic editing”, “genome editing”, “genome engineering” or the like refer to a type of genetic engineering in which DNA is inserted, deleted, modified or replaced at one or more specified locations in the genome of a cell. One key step in gene editing is creating a double stranded break at a specific point within a gene or genome. Examples of gene editing tools such as nucleases that accomplish this step include but are not limited to Zinc finger nucleases (ZFNs), transcription activator like effector nucleases (TALEN), meganucleases, and clustered regularly interspaced short palindromic repeats system (CRISPR/Cas).

As used herein, “DNA element” refers to any DNA sequence that can be transferred between cells, such as between a donor cell and a recipient cell. Thus, a DNA element includes, but is not limited to a gene, a promoter, an enhancer, a terminator, an intron, an intergenic region, a barcode, or a gRNA. A DNA element may be a fragment of a gene, a promoter, an enhancer, a terminator, an intron, an intergenic region, a barcode, or a gRNA. A DNA element may be a combination genes, promoters, enhancers, terminators, introns, intergenic regions, barcodes, gRNAs, and fragments of a genes, promoters, enhancers, terminators, introns, intergenic regions, barcodes, and gRNAs. In embodiments, the DNA element is in a donor plasmid. In other embodiments, the DNA element is moved to or is in a recipient oligonucleotide. In other embodiments, the DNA element is moved from a recipient oligonucleotide to a reset donor plasmid.

As used herein, the term “gene editing reagent” refers to components required for gene editing tools and may include enzymes, riboproteins, solutions, co-factors and the like. For example, gene editing reagents include one or more components required for Zinc finger nucleases (ZFNs), transcription activator like effector nucleases (TALEN), meganucleases, and clustered regularly interspaced short palindromic repeats system (CRISPR/Cas) gene editing.

As used herein, the term “endonuclease” refers to an enzyme or a component of an endonuclease system (e.g., any component of CRISPR, including a gRNA) which possesses endonucleolytic catalytic activity for polynucleotide cleavage. For example, an endonuclease or component thereof can cleave a phosphodiester bond of an oligonucleotide or polynucleotide. An endonuclease cleaves at a phosphodiester bond within or adjacent to its recognition site sequence, which spans at least 4 bp in length. Types of endonucleases include, but are not limited to restriction enzymes, AP endonuclease, T7 endonuclease, T4 endonuclease, Bal 31 endonuclease, Endonuclease I, Micrococcal nuclease, Endonuclease II, Neurospora endonuclease, S1 endonuclease, P1-nuclease, Mung bean nuclease I, DNAse I, RNA-guided DNA endonuclease, (e.g. CRISPR, including any CRISPR components, e.g. Cas protein, gRNA, etc.), Homothallic switching endonuclease, TALENs, zinc finger nucleases, and Endo R.

By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In some embodiments, a complex including a guide RNA and a site-specific modifying enzyme is used for targeted double-stranded DNA cleavage.

As used herein, the term “CRISPR” or “clustered regularly interspaced short palindromic repeats” is used in accordance with its plain ordinary meaning and refers to a genetic element that bacteria use as a type of acquired immunity to protect against viruses. CRISPR includes short sequences that originate from viral genomes and have been incorporated into the bacterial genome. Cas (CRISPR associated proteins) process these sequences and cut matching viral DNA sequences. Thus, CRISPR sequences function as a guide for Cas to recognize and cleave DNA that are at least partially complementary to the CRISPR sequence. By introducing plasmids including Cas genes and specifically constructed CRISPRs into eukaryotic cells, the eukaryotic genome can be cut at any desired position.

As used herein, the term “Cas9” or “CRISPR-associated protein 9” is used in accordance with its plain ordinary meaning and refers to an enzyme that uses CRISPR sequences as a guide to recognize and cleave specific strands of DNA that are at least partially complementary to the CRISPR sequence. Cas9 enzymes together with CRISPR sequences form the basis of a technology known as CRISPR-Cas9 that can be used to edit genes within organisms. This editing process has a wide variety of applications including basic biological research, development of biotechnology products, and treatment of diseases.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

A “CRISPR-associated endonuclease Cas12a,” “Cas12a,” “Cas12” or “Cas12 protein” as referred to herein includes any of the recombinant or naturally-occurring forms of the Cas12 endonuclease or variants or homologs thereof that maintain Cas12 endonuclease enzyme activity (e.g. within at least 50%, 80%, 90%, 95%, 96%, 97%, 98%, 99% or 100% activity compared to Cas12). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas12 protein. In aspects, the Cas12 protein is substantially identical to the protein identified by the UniProt reference number AOQ7Q2 or a variant or homolog having substantial identity thereto.

A “CRISPR-associated endoribonuclease Cas13a,” “Cas13a,” “Cas13” or “Cas13 protein” as referred to herein includes any of the recombinant or naturally-occurring forms of the Cas13 endoribonuclease or variants or homologs thereof that maintain Cas13 endoribonuclease enzyme activity (e.g. within at least 50%, 80%, 90%, 95%, 96%, 97%, 98%, 99% or 100% activity compared to Cas13). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13 protein. In aspects, the Cas13 protein is substantially identical to the protein identified by the UniProt reference number PODPB8 or a variant or homolog having substantial identity thereto.

As used herein, “TALEN” or “transcription activator-like effector nuclease” refers to restriction enzymes generated by attaching a DNA binding domain (e.g. a TAL effector DNA-binding domain) to a nuclease (e.g. FokI). TALEN typically includes a naturally occurring DNA-binding domain, which include multiple modules, termed TALs or TALEs. Thus, the TALs, which include variable diresidues, confer DNA binding specificity.

A “guide RNA” or “gRNA” as provided herein refers to an RNA sequence having sufficient complementarity with a target polynucleotide sequence to hybridize with the target sequence and direct sequence-specific binding of a CRISPR complex to the target sequence. For example, a gRNA can direct Cas to the target polynucleotide. In embodiments, the gRNA includes the crRNA and the tracrRNA. For example, the gRNA can include the crRNA and tracrRNA hybridized by base pairing. Thus, in embodiments, the two RNA can be encoded separately by a crRNA and tracrRNA as 2 RNA molecules which then form an RNA/RNA complex due to complementary base pairing between the crRNA and tracrRNA. In aspects, the degree of complementarity between a guide RNA sequence and its corresponding target sequence, when optimally aligned using a suitable alignment algorithm, is about 50%, 60%, 75%, 80%, 85%, 90%, 95%, 97.5%, 99%, or more. In aspects, the degree of complementarity between a guide RNA sequence and its corresponding target sequence, when optimally aligned using a suitable alignment algorithm, is at least about 50%, 60%, 75%, 80%, 85%, 90%, 95%, 97.5%, 99%.

Non-limiting examples of CRISPR enzymes include Cas1, Cas1B, Cas2, Cas3, Cas4, Cas5, Cas6, Cas7, Cas8, Cas9 (also known as Csn1 and Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In embodiments, the CRISPR enzyme is a Cas9 enzyme. In embodiments, the Cas9 enzyme is S. pneumoniae, S. pyogenes or S. thermophilus Cas9, or mutants derived thereof in these organisms. In embodiments, the CRISPR enzyme is codon-optimized for expression in a eukaryotic cell. In embodiments, the CRISPR enzyme directs cleavage of one or two strands at the location of the target sequence. In embodiments, the CRISPR enzyme lacks DNA strand cleavage activity.

As used herein, a “zinc finger” is a polypeptide structural motif folded around a bound zinc cation. In embodiments, the polypeptide of a zinc finger has a sequence of the form X₃-Cys-X₂₋₄-Cys-X₁₂-His-X₃₋₅-His-X₄, wherein X is any amino acid (e.g., X₂₋₄ indicates an oligopeptide 2-4 amino acids in length). Thus, “zinc finger nuclease” as used herein refers to a nuclease including a zinc finger motif and a domain capable of inducing breaks in the target DNA.

The term “homologous recombination” refers to a type of genetic recombination where information is exchanged between two similar or identical nucleic acid sequences, which may be referred to herein as “homology regions”. In some embodiments of the methods described here, a homology region may comprise, for example, two areas of homology which may optionally flank a non-homologous region. In embodiments of the methods described here, an E. coli RecA gene may be used for boosting homologous recombination. “RecA” refers to the bacterial homolog of the family of ubiquitous 38-kD homologous DNA repair proteins which mediates ATP-dependent homologous recombination in bacteria. In embodiments, the donor cell or recipient cell of the methods described herein includes an oligonucleotide encoding one or more homologous DNA repair genes, such as RecA. In embodiments, homologous DNA repair gene expression is inducible. In embodiments, the homologous DNA repair gene is RecA. In embodiments, homologous DNA repair genes are the recombineering genes Reda, Redo, and Red7. Non-limiting examples of methods for homologous recombination and gene editing using various nuclease systems can be found, for example, in U.S. Pat. No. 8,945,839, International PCT application Pub. No. WO2013/163394 and U.S. Patent Application Nos. 2016/0060657, 2012/0192298A1 and US2007/0042462. These and other known methods for homologous recombination can be used in combination with the methods described here.

As used herein, the term “transfection” is used in accordance with its plain ordinary meaning and refers to a process of deliberately introducing naked or purified nucleic acids into eukaryotic cells. In instances, “transfection” may refer to other methods and cell types, although other terms are often preferred. For example, the term “transformation” is typically used to describe non-viral DNA transfer in bacteria and non-animal eukaryotic cells, including plant cells. In animal cells, transfection is the preferred term. For example, the term “transduction” is often used to describe virus-mediated gene transfer into eukaryotic cells.

The terms “bacterial conjugation” and “bacterial mating” are interchangeable and refer to a mode of genetic exchange between bacteria. Typically, the bacterial conjugation involves only a portion of the genome of one of the cells (the donor) and the complete genome of its partner (the recipient cell). Thus, genetic transfer in bacterial conjugation is typically partial. In embodiments, bacterial conjugation is transfer of non-genomic bacterial DNA from a donor cell to a recipient cell. In instances, bacterial conjugation occurs through a plasmid. In instances, bacterial conjugation occurs through an exogenous DNA in the bacteria. In embodiments, the donor cell and the recipient cell are in contact for bacterial conjugation to occur. In embodiments, the donor cell and the recipient cell include linking bridge (e.g. pilus) for bacterial conjugation to occur.

In embodiments, the recipient cell or the donor cell includes an oligonucleotide that enables plasmid conjugation. In embodiments, the oligonucleotide that enables plasmid conjugation is in the donor cell genome. In embodiments, the oligonucleotide that enables plasmid conjugation is in a helper plasmid. In embodiments, the oligonucleotide that enables plasmid conjugation is the Tra operon. In embodiments, the oligonucleotide that enables plasmid conjugation is selected from: IncF1 Tra (traA, traB, traC, traD, traE, traF, traG, traH, traI, traJ, traK, traL, traM, traN, traO, traP, traQ, traR, traS, traT), IncP Tra operon: (trbA, trbB, trbC, trbD, trbE, trbF, trbG, trbH, trbI, trbJ, trbK, trbL, traA, traB, traC, traD, traE, traF, traG, traH, traI, traJ, traK, traL, traM, traN, traO), IncIl tra operon: (traE, traF, traG, traH, traI, traJ, traK, traL, traM, traN, traO, traP, traQ, traS, traT, traU, traV, traW, traY), pTiC58 tra genes: (traA, traF, traB, traC, traG, traD, traR, traI), and pIJ101: clt, korB. In embodiments, the oligonucleotide that enables plasmid conjugation is IncF1 Tra (traA, traB, traC, traD, traE, traF, traG, traH, traI, traJ, traK, traL, traM, traN, traO, traP, traQ, traR, traS, traT). In embodiments, the oligonucleotide that enables plasmid conjugation is IncP Tra operon: (trbA, trbB, trbC, trbD, trbE, trbF, trbG, trbH, trbI, trbJ, trbK, trbL, traA, traB, traC, traD, traE, traF, traG, traH, traI, traJ, traK, traL, traM, traN, traO). In embodiments, the oligonucleotide that enables plasmid conjugation is IncIl tra operon: (traE, traF, traG, traH, traI, traJ, traK, traL, traM, traN, traO, traP, traQ, traS, traT, traU, traV, traW, traY). In embodiments, the oligonucleotide that enables plasmid conjugation is pTiC58 tra genes: (traA, traF, traB, traC, traG, traD, traR, traI). In embodiments, the oligonucleotide that enables plasmid conjugation is pIJ101: clt, korB.

As used herein, “donor cell” refers to a cell (e.g. a bacteria cell) that transfers genetic material to another cell (e.g. a bacteria cell, a plant cell, etc.). The cell that receives the transferred genetic material is referred to herein as a “recipient cell”.

The term “donor plasmid”, as used herein refers to DNA from a donor cell (e.g. a bacteria cell) including an oligonucleotide sequence (e.g. donor DNA, oligonucleotide including a DNA element) that is to be transferred from the donor cell to a recipient cell (e.g. a bacteria cell, yeast cell, plant cell, etc.). Typically, the donor plasmid is a circular double-stranded DNA that is separate from genomic DNA. Thus, the term “recipient plasmid” refers to DNA from a recipient cell that receives the donor DNA. In embodiments, the DNA from the donor plasmid is received by DNA other than DNA from a recipient plasmid. Thus, in embodiments, the donor DNA may be incorporated into genomic DNA.

In embodiments, the donor plasmid includes an origin of transfer. In embodiments, the origin of transfer is from a mobile element. In embodiments, the mobile element is a plasmid. In embodiments, the plasmid is an IncFI plasmid, an IncPα plasmid, an IncI1 plasmid, a pTiC58 from Agrobacterium tumefaciens, a pAD1 plasmid, an Inc18 plasmid, or an IncH plasmid. In embodiments, the plasmid is an IncFI plasmid. In embodiments, the plasmid is an IncPa plasmid. In embodiments, the plasmid is an IncI1 plasmid. In embodiments, the plasmid is a pTiC58 from Agrobacterium tumefacien. In embodiments, the plasmid is a pAD1 plasmid. In embodiments, the plasmid is an Inc18 plasmid. In embodiments, the plasmid is an IncH plasmid. The plasmids are discussed in greater detail in Ippen-Ihler, K. A., and Minkley, E. G., Jr., (1986). The conjugation system of F, the fertility factor of Escherichia coli. Ann. Rev. Genet. 20:593-624.; Guiney, D. G., and Lanka, E., (1989), Conjugative transfer of IncP plasmids, in: Promiscuous Plasmids of Gram-negative Bacteria (C. M. Thomas, ed.), Academic Press, London, pp. 27-56.; Catherine E. D. Rees, David E. Bradley, Brian M. Wilkins, (1987) Organization and regulation of the conjugation genes of IncI1 plasmid ColIb-P9. Plasmid. 18: 223-236.; von Bodman S B, McCutchan J E, Farrand S K. (1989) Characterization of conjugal transfer functions of Agrobacterium tumefaciens Ti plasmid pTiC58. J. Bacteriol. 171(10):5281-5289.; Clewell D B, Weaver K E. (1989) Sex pheromones and plasmid transfer in Enterococcus faecalis. Plasmid. 21(3):175-84.; Kohler V, Vaishampayan A, Grohmann E. (2018) Broad-host-range Inc18 plasmids: Occurrence, spread and transfer mechanisms. Plasmid. 99:11-21.; Andreas Schlüter, Patrice Nordmann, Rémy A. Bonnin, Yves Millemann, Felix G. Eikmeyer, Daniel Wibberg, Alfred Pühler, Laurent Poirel. (2014) IncH-Type Plasmid Harboring bla_{CTX-M-15}, bla_{DHA-1}, and qnrB4 Genes Recovered from Animal Isolates. Antimicrobial Agents and Chemotherapy 58(7):3768-3773. The entire contents of these references are incorporated herein by reference in their entirety for all purposes.

In embodiments, the origin of transfer is from a mobile element. In embodiments, the mobile element is from a conjugative transposon. In embodiments, the conjugative transposon is Tn916 from Enterococcus faecalis or CTnDOT from Bacteroides. In embodiments, the conjugative transposon is Tn916 from Enterococcus faecalis. In embodiments, the conjugative transposon is CTnDOT from Bacteroides. In embodiments, the mobile element is from an integrating conjugative element. In embodiments, the mobile element is from SXT from Vibrio cholerae or R391 from Providencia rettgeri. In embodiments, the mobile element is from SXT from Vibrio cholerae. In embodiments, the mobile element is from R391 from Providencia rettgeri. The elements are described in more detail in references Rice L. B. (1998). Tn916 family conjugative transposons and dissemination of antimicrobial resistance determinants. Antimicrobial agents and chemotherapy, 42(8), 1871-1877.; Cheng Q, Paszkiet B J, Shoemaker N B, Gardner J F, Salyers A A. (2000) Integration and excision of a Bacteroides conjugative transposon, CTnDOT. J Bacteriol. 182(14):4035-43.; Bianca Hochhut and Matthew K. Waldor. (1999) Site-specific integration of the conjugal Vibrio cholerae SXT element into prfC. Mol. Microbiology. 32(1):99-110.; Böltner D, MacMahon C, Pembroke J T, Strike P, Osborn A M. R391: a conjugative integrating mosaic comprised of phage, plasmid, and transposon elements. J Bacteriol. 2002; 184(18):5158-5169., each of which is herein incorporated by reference in its entirety.

In embodiments, the donor plasmid includes a conditional replication origin. In embodiments, the conditional replicon is R6K-pir, RSF1010 oriV-RepA/B/C, ColE2 P9-RepA, RP4 oriV-trfA, pPS10 oriV-RepA, pSC101 ori-RepC^{TS}, RK2 oriV, bacteriophage P1 ori, plasmid pSC101 origin of replication, bacteriophage lambda ori, pBR322 plasmid, pSU739 plasmid, or pSU300 plasmid. In embodiments, the conditional replicon is R6K-pir. In embodiments, the conditional replicon is RSF1010 oriV-RepA/B/C. In embodiments, the conditional replicon is ColE2 P9-RepA. In embodiments, the conditional replicon is RP4 oriV-trfA. In embodiments, the conditional replicon is pPS10 oriV-RepA. In embodiments, the conditional replicon is pSC101 ori-RepC^{TS}. In embodiments, the conditional replicon is RK2 oriV. In embodiments, the conditional replicon is bacteriophage P1 ori. In embodiments, the conditional replicon is plasmid pSC101 origin of replication. In embodiments, the conditional replicon is bacteriophage lambda ori. In embodiments, the conditional replicon is pBR322 plasmid. In embodiments, the conditional replicon is pSU739 plasmid. In embodiments, the conditional replicon is pSU300 plasmid. The plasmids are described in references: Metcalf W W, Jiang W, Daniels L L, Kim S K, Haldimann A, Wanner B L. (1996) Conditionally replicative and conjugative plasmids carrying lacZ alpha for cloning, mutagenesis, and allele replacement in bacteria. Plasmid. 35(1):1-13.; Scherzinger E, Bagdasarian M M, Scholz P, Lurz R, Ruckert B, Bagdasarian M. (1984) Replication of the broad host range plasmid RSF1010: requirement for three plasmid-encoded proteins. Proc Natl Acad Sci USA.81(3):654-8.; ColE2-P9: Yagura M, Nishio S Y, Kurozumi H, Wang C F, Itoh T. (2006) Anatomy of the replication origin of plasmid ColE2-P9. J Bacteriol. 188(3):999-1010.; Ayres E K, Thomson V J, Merino G, Balderes D, Figurski D H. Precise deletions in large bacterial genomes by vector-mediated excision (VEX). (1993) The trfA gene of promiscuous plasmid RK2 is essential for replication in several gram-negative hosts. J Mol Biol. 5; 230(1):174-85.; Maestro B, Sanz J M, Díaz-Orejas R, Fernández-Tresguerres E. (2003) Modulation of pPS10 host range by plasmid-encoded RepA initiator protein. J Bacteriol. 185(4):1367-75.; Hashimoto-Gotoh, T., & Sekiguchi, M. (1977). Mutations of temperature sensitivity in R plasmid pSC101. Journal of bacteriology, 131(2), 405-412.; Ayres E K, Thomson V J, Merino G, Balderes D, Figurski D H. Precise deletions in large bacterial genomes by vector-mediated excision (VEX). (1993) The trfA gene of promiscuous plasmid RK2 is essential for replication in several gram-negative hosts. J Mol Biol. 5; 230(1):174-85. Stenzel T T, Patel P, Bastia D. (1987) The integration host factor of Escherichia coli binds to bent DNA at the origin of replication of the plasmid pSC101. Cell. 5; 49(5):709-17.; Sugiura S, Ohkubo S, Yamaguchi K. (1993) Minimal essential origin of plasmid pSC101 replication: requirement of a region downstream of iterons. J Bacteriol. 175(18):5993-6001.; Pal S K, Mason R J, Chattoraj D K. (1986) P1 plasmid replication. Role of initiator titration in copy number control. J Mol Biol. 20; 192(2):275-85.; LeBowitz J H, McMacken R. (1984) The bacteriophage lambda O and P protein initiators promote the replication of single-stranded DNA. Nucleic Acids Res. 12(7):3069-3088.; Grindley N D, Kelley W S. (1976) Effects of different alleles of the E. coli K12 pol A gene on the replication of non-transferring plasmids. Mol Gen Genet. 2; 143(3):311-8.; Francia, M. V., & García Lobo, J. M. (1996). Gene integration in the Escherichia coli chromosome mediated by Tn21 integrase (Int21). Journal of bacteriology, 178(3), 894-898.; Mendiola M V, de la Cruz F. (1989) Specificity of insertion of IS91, an insertion sequence present in alpha-haemolysin plasmids of Escherichia coli. Mol Microbiol. 3(7):979-84. The references are incorporated herein in their entirety.

In embodiments, the conditional replication origin is dependent on presence of an oligonucleotide. In embodiments, the oligonucleotide encodes pir1, pir1-116, repA/repB/repC (RSF1010 replicon), repA (ColE2-P9 replicon), trfA (RP4 replicon), RepA (pSP10 replicon), RepC^{TS} (pSC101 replicon), or a combination thereof. In embodiments, the oligonucleotide encodes pir1. In embodiments, the oligonucleotide encodes pir1-116. In embodiments, the oligonucleotide encodes repA/repB/repC (RSF1010 replicon). In embodiments, the oligonucleotide encodes repA (ColE2-P9 replicon). In embodiments, the oligonucleotide encodes trfA (RP4 replicon). In embodiments, the oligonucleotide encodes RepA (pSP10 replicon). In embodiments, the oligonucleotide encodes RepC^{TS} (pSC101 replicon).

In embodiments, the conditional replication origin depends on a condition of cell growth. In embodiments, the condition is temperature.

For the methods provided herein, in embodiments, the donor plasmid or recipient oligonucleotide includes a replicon that can replicate plasmids of lengths from 20 or 30 kilobases.

For the methods provided herein, in embodiments, the donor plasmid or recipient oligonucleotide includes a replicon that that can replicate plasmids of lengths greater than 30 kilobases. In embodiments, the replicon can replicate plasmids of lengths of about 30 kilobases to about 500 kilobases. In embodiments, the replicon can replicate plasmids of lengths of about 30 kilobases. In embodiments, the replicon can replicate plasmids of lengths of about 50 kilobases. In embodiments, the replicon can replicate plasmids of lengths of about 70 kilobases. In embodiments, the replicon can replicate plasmids of lengths of about 90 kilobases. In embodiments, the replicon can replicate plasmids of lengths of about 100 kilobases. In embodiments, the replicon can replicate plasmids of lengths of about 120 kilobases. In embodiments, the replicon can replicate plasmids of lengths of about 140 kilobases. In embodiments, the replicon can replicate plasmids of lengths of about 160 kilobases. In embodiments, the replicon can replicate plasmids of lengths of about 180 kilobases. In embodiments, the replicon can replicate plasmids of lengths of about 200 kilobases. In embodiments, the replicon can replicate plasmids of lengths of about 220 kilobases. In embodiments, the replicon can replicate plasmids of lengths of about 240 kilobases. In embodiments, the replicon can replicate plasmids of lengths of about 260 kilobases. In embodiments, the replicon can replicate plasmids of lengths of about 280 kilobases. In embodiments, the replicon can replicate plasmids of lengths of about 300 kilobases. In embodiments, the replicon can replicate plasmids of lengths of about 400 kilobases. In embodiments, the replicon can replicate plasmids of lengths of about 500 kilobases. The length may be any value or subrange within the indicated ranges, including endpoints.

In embodiments, the replicon is from a P1-derived artificial chromosome or a bacterial artificial chromosome. In embodiments, the replicon is from a P1-derived artificial chromosome. In embodiments, the replicon is from a bacterial artificial chromosome. In embodiments, the donor plasmid or recipient oligonucleotide includes an inducible high-copy replication of origin. In embodiments, the donor plasmid includes an inducible high-copy replication of origin. In embodiments, the recipient oligonucleotide includes an inducible high-copy replication of origin.

In embodiments, the donor plasmid or recipient oligonucleotide is a yeast artificial chromosome (YAC), a mammalian artificial chromosome (MAC), a human artificial chromosome (HAC), or a plant artificial chromosome. In embodiments, the donor plasmid is a yeast artificial chromosome (YAC). In embodiments, the donor plasmid is a mammalian artificial chromosome (MAC). In embodiments, the donor plasmid is a human artificial chromosome (HAC). In embodiments, the donor plasmid is a plant artificial chromosome. In embodiments, the recipient oligonucleotide is a yeast artificial chromosome (YAC). In embodiments, the recipient oligonucleotide is a mammalian artificial chromosome (MAC). In embodiments, the recipient oligonucleotide is a human artificial chromosome (HAC). In embodiments, the recipient oligonucleotide is a plant artificial chromosome.

In embodiments, the donor plasmid or recipient oligonucleotide comprises a conjugation competent vector, which may be a viral vector. In embodiments, the donor plasmid is a viral vector. In embodiments, the recipient oligonucleotide is a viral vector. In embodiments, the viral vector is a retrovirus. In embodiments, the viral vector is a lentivirus. In embodiments, the viral vector is an adenovirus. In embodiments, the viral vector is an adeno-associated virus. In embodiments, the viral vector is a tobacco mosaic virus. In embodiments, the viral vector is a baculovirus. In embodiments, the viral vector is a herpes simplex virus. In embodiments, the viral vector is a poxvirus. In embodiments, the viral vector is gammaretrovirus. In embodiments, the viral vector is Sendai virus.

As used herein, the term “control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

As used herein, the terms “origin of transfer” or “oriT” refer to a short sequence (up to 500 bp) that is necessary for transfer of DNA from a bacterial host and recipient during bacterial conjugation.

As used herein, “curable origin of replication” refers to an origin of replication that does not replicate when cells are grown in the presence of certain chemicals or environmental conditions. Under these conditions, plasmids containing a curable origin of replication are lost from the cell. For example, pSC101 ori^{TS} does not function at high temperature and is lost.

As used herein, the term “mobile element” is a type of genetic material that can move around within a genome, or can be transferred between genomes, even between species.

As used herein, the term “conjugative transposon” refers to integrated DNA elements that excise themselves to form a covalently closed circular intermediate that can be reintegrated in the same cell or transferred via conjugation to a recipient cell.

As used herein, the term “integrating conjugative element” refers to a group of chromosomally integrated, self-transmissible genetic elements.

As used herein, the term “P1-derived artificial chromosome” refers to a DNA construct that originated from the P1 bacteriophage.

As used herein, the term “bacterial artificial chromosome” refers to an engineered DNA sequence used to clone DNA sequences into bacteria.

The term “recombination-mediated genetic engineering genes” or “recombineering genes” refers to genes that assist in creating genetic modifications in a DNA sequence. In instances, recombination-mediated genetic engineering genes allow in vivo construction of constructions in cells (e.g. bacteria cells) without in vitro genetic engineering techniques. In instances, recombination-mediated genetic engineering genes allow for genetic modifications to occur without introduction of enzymes including ligases and restriction enzymes. For example, the genes may be involved in a bacterium's natural process of homologous recombination without traditional molecular biology techniques known in the art. In embodiments, the recombineering genes are lambda red genes. The recombineering genes are Reda, Redo, and Redy. For example, the genes may induce homologous recombination at a high rate in bacteria. In a donor cell or recipient cell, expression of one or more recombineering genes may be inducible. In embodiments, the donor cell or the recipient cell includes an oligonucleotide encoding one or more recombination-mediated genetic engineering genes. In embodiments, the oligonucleotide encoding one or more recombination-mediated genetic engineering genes is in the donor cell plasmid. In embodiments, the recombination-mediated genetic engineering genes are inducible. In embodiments, the recombination-mediated genetic engineering genes are Reda, Redo, and Redy. In embodiments, the oligonucleotide encoding one or more homologous DNA repair genes is in the recipient cell genome. In embodiments, the oligonucleotide encoding one or more homologous DNA repair genes is in a helper plasmid. In embodiments, the oligonucleotide encoding one or more homologous DNA repair genes is in the recipient oligonucleotide, which may be in the form of a plasmid. In embodiments, the oligonucleotide encoding one or more homologous DNA repair genes is in the recipient cell genome.

As used herein, the term “inducible high-copy replication of origin” refers to a plasmid or vector containing a high number of origins of replication, (for example, between 150-200 copies in E. coli plasmid pUC) that are inducible by environmental conditions, such as change in temperature.

As used herein, the term “helper plasmid” is a plasmid that contains genes or other DNA elements necessary for a bacteria to carry out a specified function. A helper plasmid may contain an endonuclease, elements to transfer foreign DNA into the genome, to transfer a plasmid to another cell, or to perform homologous recombination. In embodiments, the helper plasmid is an IncF1 plasmid, an IncPa plasmid, an IncIl plasmid, a pTiC58 from Agrobacterium tumefaciens, a cAD1 plasmid, an Inc18 plasmid, a pIJ101 from Streptomyces, or an IncH plasmid. In embodiments, the helper plasmid is an IncF1 plasmid. In embodiments, the helper plasmid is an IncPa plasmid. In embodiments, the helper plasmid is an IncIl plasmid. In embodiments, the helper plasmid is a pTiC58 from Agrobacterium tumefaciens. In embodiments, the helper plasmid is a cAD1 plasmid. In embodiments, the helper plasmid is an Inc18 plasmid. In embodiments, the helper plasmid is a pIJ101 from Streptomyces. In embodiments, the helper plasmid is an IncH plasmid. In embodiments, the helper plasmid lacks a functional origin of transfer. In embodiments, the helper plasmid includes a selectable marker selecting for retention of the helper plasmid in the donor cell.

As used herein, the term “homing endonuclease” refers to an endonuclease that is either encoded as a free-standing gene with in an intron sequence, as a fusion with a host protein, or as a self-splicing protein. Homing endonucleases catalyze the hydrolysis of DNA at longer recognition sites, when compared to Group II restriction enzymes. Homing endonuclease examples include, but are not limited to, LAGLIDAG, GIY-YIG, His-Cys box, H-N-H, PD-(D/E)xK, and Vsr-like/EDxHD. In embodiments, the homing endonuclease is I-ScaI, PI-SceI, I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-SceI, I-SceII, I-SceIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In embodiments, the homing endonuclease is I-ScaI. In embodiments, the homing endonuclease is PI-SceI. In embodiments, the homing endonuclease is I-AniI. In embodiments, the homing endonuclease is I-CeuI. In embodiments, the homing endonuclease is I-ChuI. In embodiments, the homing endonuclease is I-CpaI. In embodiments, the homing endonuclease is I-CpaII. In embodiments, the homing endonuclease is I-CreI. In embodiments, the homing endonuclease is I-DmoI. In embodiments, the homing endonuclease is H-DreI. In embodiments, the homing endonuclease is I-HmuI. In embodiments, the homing endonuclease is I-HmuII. In embodiments, the homing endonuclease is I-LlaI. In embodiments, the homing endonuclease is I-MsoI. In embodiments, the homing endonuclease is PI-PfuI. In embodiments, the homing endonuclease is PI-PkoII. In embodiments, the homing endonuclease is I-PorI. In embodiments, the homing endonuclease is I-PpoI. In embodiments, the homing endonuclease is PI-PspI. In embodiments, the homing endonuclease is I-SceI. In embodiments, the homing endonuclease is I-SceII. In embodiments, the homing endonuclease is I-SceIII. In embodiments, the homing endonuclease is I-SceIV. In embodiments, the homing endonuclease is I-SceV. In embodiments, the homing endonuclease is I-SceVI. In embodiments, the homing endonuclease is I-SceVII. In embodiments, the homing endonuclease is I-Ssp6803I. In embodiments, the homing endonuclease is I-TevI. In embodiments, the homing endonuclease is I-TevII. In embodiments, the homing endonuclease is I-TevIII. In embodiments, the homing endonuclease is PI-TliI. In embodiments, the homing endonuclease is PI-TliII. In embodiments, the homing endonuclease is I-Tsp061I, or I-Vdi141I. In embodiments, the homing endonuclease is I-Vdi141I.

As used herein, the term “RNA-guided DNA endonuclease” refers to any DNA endonuclease that is guided to a target DNA sequence by a helper or guide RNA molecule. Examples of an RNA-guided DNA endonuclease include, but are not limited to, Cas1, Cas1B, Cas2, Cas3, Cas4, Cas5, Cas6, Cas7, Cas8, Cas9 (also known as Csn1 and Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, all variants and homologs thereof.

As used herein, the term “HO” or “Homothallic switching endonuclease” refers to the zinc-finger nuclease in Saccharomyces cerevisiae responsible for initiation of mating type interconversion.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, “donor cell” refers to a cell (e.g. a bacteria cell) that transfers genetic material to another cell (e.g. a bacteria cell, a plant cell, etc.). The cell that receives the transferred genetic material is referred to herein as a “recipient cell”.

The term “donor plasmid”, as used herein refers to DNA from a donor cell (e.g. a bacteria cell) including an oligonucleotide sequence (e.g. donor DNA, oligonucleotide including a DNA element or fragment thereof) that is to be transferred from the donor cell to a recipient cell (e.g. a bacteria cell, yeast cell, plant cell, etc.). Typically, the donor plasmid is a circular double-stranded DNA that is separate from genomic DNA. Thus, the term “recipient oligonucleotide” may refer to a plasmid DNA in a recipient cell that receives the donor DNA (e.g. by homologous recombination of the donor DNA into the recipient); or the term “recipient oligonucleotide” may refer to any oligonucleotide in the recipient cell that receives the donor DNA, for example genomic DNA of the recipient cell. In embodiments, the DNA from the donor plasmid is received by DNA other than DNA from a recipient plasmid. Thus, in embodiments, the donor DNA may be incorporated into genomic DNA.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

In embodiments of the methods described here, the donor cell or recipient cell includes an oligonucleotide encoding one or more homologous DNA repair genes. In embodiments, the oligonucleotide encoding the one or more homologous DNA repair genes is in the first, second, or subsequent donor plasmid. In embodiments, homologous DNA repair gene expression is inducible. In embodiments, the homologous DNA repair gene is RecA.

In embodiments of the methods described here, the donor cell or recipient cell includes an oligonucleotide encoding one or more recombination-mediated genetic engineering genes. In embodiments, the oligonucleotide encoding one or more recombination-mediated genetic engineering genes is in the donor cell plasmid.

In embodiments of the methods described here, the recombination-mediated genetic engineering genes are inducible. In embodiments, the recombination-mediated genetic engineering genes are Reda, Redo, and Redy. In embodiments, the oligonucleotide encoding one or more homologous DNA repair genes is in the recipient cell genome. In embodiments, the oligonucleotide encoding one or more homologous DNA repair genes is in a helper plasmid. In embodiments, the oligonucleotide encoding one or more homologous DNA repair genes is in the recipient oligonucleotide, which may be in the form of a plasmid. In embodiments, the oligonucleotide encoding one or more homologous DNA repair genes is in the recipient cell genome.

In embodiments of the methods described here, donor cells, recipient cells, or recombinant recipient cells may be in an ordered array, or in a first or second ordered array. In embodiments, the donor cells, recipient cells, or recombinant recipient cells may be transferred to positions on a third ordered array, a fourth ordered array, or a subsequent ordered array.

In embodiments of the methods described here, the donor cells and the recipient cells are bacteria cells. In embodiments, the recipient cells are not bacteria cells. In embodiments, the recipient cells are plant cells. In embodiments, the recipient cells are yeast cells. In embodiments, the recipient cells are mammalian cells.

### II. Methods of Assembling a DNA Element

The invention provides methods of assembling a plurality of DNA elements into an assembled DNA element within a recipient cell, the method comprising: (a) contacting a first donor cell comprising a first donor plasmid with a recipient cell comprising a recipient oligonucleotide under conditions to (i) transfer the first donor plasmid from the first donor cell to the recipient cell by conjugation and (ii) recombine the first donor plasmid and the recipient oligonucleotide in the recipient cell by homologous recombination wherein the first donor plasmid comprises, in sequential order, an optional first endonuclease site (C1), a first homologous recombination region (HR1), a first oligonucleotide comprising a first DNA element fragment (oligo1), a second homologous recombination region (HR2) comprising two homologous recombination regions (HR2.1, HR2.2) and an optional third endonuclease site (C3); the recipient oligonucleotide comprises a third homologous recombination region (HR3) homologous to HR1 and a fourth homologous recombination region (HR4) homologous to HR2.2; thereby providing, following the homologous recombination of HR1 with HR3 and HR2.2 with HR4, a first recombined recipient oligonucleotide comprising the first DNA element fragment; (b) contacting a second donor cell comprising a second donor plasmid with the recipient cell comprising the first recombined recipient oligonucleotide under conditions to (i) transfer the second donor plasmid from the second donor cell to the first recipient cell by conjugation and (ii) recombine the second donor plasmid and the first recombined recipient oligonucleotide to form a second recombined recipient oligonucleotide in the recipient cell by homologous recombination; wherein the second donor plasmid comprises, in sequential order, an optional fifth endonuclease site (C5), a fifth homologous recombination region (HR5) homologous to HR2.1, a second oligonucleotide encoding a second DNA element fragment (oligo2), a sixth homologous recombination region (HR6) comprising two homologous recombination regions (HR6.1, HR6.2), and an optional sixth endonuclease site (C6); thereby providing, following the homologous recombination of HR5 with HR2.1 and HR6.2 with HR4, a second recombined recipient oligonucleotide comprising the first and second DNA element fragments (oligo1, oligo2), which form a DNA assembly. In embodiments, HR2.1 and HR2.2 flank a non-homologous region comprising one (C2) or two endonuclease sites (C2.1, C2.2);
optionally wherein HR3 and HR4 flank a non-homologous region comprising one (C4) or two endonuclease sites (C4.1, C4.2). In embodiments, HR6.1 and HR6.2 flank a non-homologous region comprising one (C7) or two endonuclease sites (C7.1, C7.2). In embodiments, the recipient oligonucleotide is in a recipient cell plasmid or the recipient cell genome. In embodiments, the DNA assembly comprises at least a portion of a gene, a promoter, an enhancer, a terminator, an intron, an intergenic region, a barcode, a guide RNA (gRNA), or a combination thereof. In embodiments, step (b) is repeated for one or more iterations with a third or subsequent donor cell comprising a third or subsequent donor plasmid comprising compatible HR regions and a third or subsequent oligonucleotide encoding a third or subsequent DNA element fragment (oligo3, oligo4, . . . oligoN), thereby forming a third or subsequent recombined recipient oligonucleotide comprising the first, the second, and a third or subsequent DNA element fragments which together form a DNA assembly. In embodiments, step (a) comprises a plurality of first donor cells, each comprising a different first donor plasmid; and step (b) comprises a plurality of second, third, or subsequent donor cells, each comprising a different second, third, or subsequent donor plasmid; optionally wherein each first donor cell is in a position in a first ordered array and each second, third, or subsequent donor cell is in a position in a second, third, or subsequent ordered array; optionally wherein the method generates a combinatorial library comprising a plurality of different assembled DNA elements.

In embodiments, the donor plasmid comprising the last DNA element to form part of an assembled DNA element comprises a barcode homologous recombination (BHR) region to produce recipient cells each containing a recombined recipient oligonucleotide comprising the assembled DNA element the BHR, and a further HR; and the method further comprises (i) constructing or acquiring an array of barcode donor cells, each containing a barcode donor plasmid comprising an HR homologous to the BHR, a unique barcode oligonucleotide, and a second HR homologous to the further HR of the recombined recipient oligonucleotide; (ii) contacting the array of barcode donor cells with an array of the recipient cells under conditions to (a) transfer the barcode donor plasmids from the barcode donor cells to the recipient cells by conjugation and (b) recombine the barcode donor plasmids and recipient oligonucleotides in the recipient cells by homologous recombination, thereby producing an array of recipient cells comprising barcoded assemblies.

In embodiments, each donor plasmid comprises a further pair of unique endonuclease sites CX, CY, flanking a barcode homologous recombination (BHR) region and the method further comprises contacting an array of recipient cells, each comprising a DNA assembly, with an array of barcode donor cells, each containing a barcode donor plasmid comprising a pair of HR regions homologous to the BHR flanking a unique barcode oligonucleotide, to produce an array of recipient cells comprising barcoded assemblies.

In embodiments, the DNA assembly methods may further comprise contacting a reset donor cell comprising a reset donor plasmid with a recipient cell comprising a recombined recipient oligonucleotide, wherein the reset donor plasmid comprises, in sequential order, a homologous recombination region (HRt) homologous to a terminal sequence of the DNA assembly, a reset endonuclease site, a selectable marker, a reset endonuclease site, a homologous recombination region (HRX), and an origin of transfer, wherein the recombined recipient oligonucleotide comprises, in sequential order, a reset endonuclease site, the DNA assembly, a homologous recombination region homologous to HRX (HRXa) and a reset endonuclease site, thereby providing, subsequent to homologous recombination between the HRt and the terminal sequence of the DNA assembly and between the HRX and the HRXa, a reset plasmid comprising the origin of transfer and the DNA assembly. In embodiments, the reset plasmid is in a donor cell. In embodiments, the reset plasmid contains a restricted origin of replication that functions in both donor cells and recipient cells. In embodiments, the reset donor plasmid is constructed by a method comprising introducing an oligonucleotide insert comprising homologous recombination regions HRt, HRX, flanking two endonuclease sites (C1, C2) and a counter-selectable marker (CM), HRt-C1-CM-C2-HRX; or a library of such oligonucleotide inserts; allowing an endonuclease to cleave the endonuclease sites and introducing a counter-selectable marker at the cleavage sites using homologous recombination.

The invention also provides methods of conjugating barcodes to oligonucleotides, the method comprising (a) inserting each oligonucleotide of a mixture of oligonucleotides into a donor plasmid, each donor plasmid comprising, in sequential order, optionally a first endonuclease site (C1), a first homologous recombination region (HR1), a second homologous recombination region (HR2), and optionally a second endonuclease site (C2); wherein each oligonucleotide is inserted between HR1 and HR2, thereby providing a plurality of donor plasmids comprising donor oligonucleotides, each donor plasmid comprising a single donor oligonucleotide from the mixture of oligonucleotides: C1-HR1-oligo-HR2-C2; (b) transforming a plurality of cells with the plurality of donor plasmids such that each cell comprises a donor plasmid, thereby forming a plurality of donor cells; (c) plating and culturing the plurality of donor cells, each in a unique position on a first ordered array, thereby providing a first ordered array of donor cells; (d) providing a plurality of recipient cells in a second ordered array, wherein each recipient cell comprises a recipient oligonucleotide comprising, in sequential order, a unique barcode sequence, wherein the unique barcode sequence identifies a position of the recipient cell in the second ordered array, a third homologous recombination region (HR3) homologous to HR1, optionally a third endonuclease site (C3), and a fourth homologous recombination region (HR4) homologous to HR2; (e) contacting the first ordered array of donor cells with the second ordered array of recipient cells under conditions to (i) transfer the donor plasmids from the donor cells to the recipient cells in corresponding positions on the array by conjugation, (ii) optionally cleave the first, second, and third endonuclease sites, and (ii) transfer the oligonucleotides from the donor plasmids to the recipient cell oligonucleotides by homologous recombination, thereby forming an third array of fusion oligonucleotides, each comprising a unique barcode sequence and a donor oligonucleotide from the mixture of oligonucleotides; and (f) optionally sequencing the fusion oligonucleotides, and thereby identifying each oligonucleotide in the array of by its barcode sequence. In embodiments, the recipient oligonucleotide is in a recipient cell plasmid or the recipient cell genome. In embodiments, the donor plasmid comprises a selectable marker between HR1 and HR2 selecting for integration of the oligonucleotide into the recipient cell oligonucleotide; optionally wherein the donor plasmid comprises a counter-selectable marker. In embodiments, the recipient cell oligonucleotide comprises a fourth endonuclease site (C4).

In a further aspect is provided a method of assembling a DNA element. The method includes: (a) providing a first host cell including a first donor plasmid including, in sequential order: (i) a first endonuclease target site, (ii) a first homologous recombination region, (iii) optionally a first oligonucleotide including a first DNA element fragment, (iv) a second homologous recombination region, (v) a second endonuclease target site, (vi) and a third endonuclease target site, (b) providing a recipient cell, wherein the recipient cell includes a recipient oligonucleotide including: (i) a third homologous recombination region, wherein the third homologous region is homologous to the first homologous recombination region, (ii) a fourth endonuclease target site, and (iii) a fourth homologous region, wherein the fourth homologous recombination region is homologous to the second homologous recombination region; and (c) contacting the first host cell with the recipient cell under conditions to (i) transfer the first donor plasmid from the first host cell to the recipient cell by bacterial conjugation, (ii) direct a first endonuclease to the at least one of the first endonuclease target site, the third endonuclease target site, or the fourth endonuclease target site, thereby producing double-stranded breaks in the first donor plasmid and the recipient oligonucleotide and (iii) recombine the first donor plasmid and the recipient oligonucleotide in the recipient cell by homologous recombination via the first and second homologous recombination regions with the third and fourth corresponding homologous recombination regions, thereby forming a recombined recipient oligonucleotide. The method may further include: (d) providing a second host cell including a second donor plasmid including, in sequential order: (i) a fifth endonuclease target site, (ii) a fifth homologous recombination region which is homologous to the second homologous region, (iii) a second oligonucleotide including a second DNA element fragment, (iv) a sixth homologous recombination region which is homologous to the fourth homologous region, and (v) a sixth endonuclease target site; (e) contacting the second host cell with the recipient cell containing the recombined recipient oligonucleotide under conditions to (i) transfer the second donor plasmid from the second host cell to the recipient cell by bacterial conjugation, (ii) express a second endonuclease, (iii) direct the second endonuclease to the second endonuclease target site, the fifth endonuclease target site and/or the sixth endonuclease target site, thereby producing double-stranded breaks, (iv) recombine the second donor plasmid and the recombined recipient oligonucleotide in the recipient cell by homologous recombination via the fifth and sixth homologous recombination sites with the corresponding second and fourth homologous recombination sites, thereby forming a second recombined recipient oligonucleotide including an assembled DNA element. In embodiments, the a different portion of the fourth homologous region is homologous to the sixth homologous recombination region, compared to the portion of the fourth homologous region that is homologous to the second homologous recombination region.

In embodiments, step (a) includes a plurality of first host cells, wherein each cell includes a unique first oligonucleotide. In embodiments, step (d) includes a plurality of second host cells, wherein each cell includes a unique second oligonucleotide. In embodiments, each first host cell includes a unique plasmid. In embodiments, each second host cells includes a unique plasmid. In embodiments, each first host cell is in a position in a first ordered array. In embodiments, a plurality of first host cells are in a position in a first ordered array, thereby forming a pool of first host cells in the first ordered array. In embodiments, each second host cell is in a position in a second ordered array. In embodiments, a plurality of second host cells are in a position in a second ordered array, thereby forming a pool of second host cells in each position in the second ordered array.

In embodiments, the first donor cell is in a first ordered array, the second donor cell is in a second ordered array, and one or more subsequent donor cells are in one or more subsequent arrays. Thus, in embodiments, the method provided herein generates a variant library including a plurality of different assembled DNA elements. In embodiments, the variant library is generated by 1) making each variant independently using a first host cell, a second host cell, or a subsequent host cell in a position in a first, second array, or subsequent array or 2) generating a variant pool using a plurality of first host cells, second host cells, or subsequent host cells in a position in a first, second array, or subsequent array. In embodiments, the variant library is generated by making each variant independently using a first host cell, a second host cell, or a subsequent host cell in a position in a first, second array, or subsequent array. In embodiments, the variant library is generated by generating a variant pool using a plurality of first host cells, second host cells, or subsequent host cells in a position in a first, second array, or subsequent array. For example, for the method provided herein including embodiments thereof, the first DNA element and/or second DNA element may be a DNA barcode or plurality of DNA barcodes. In embodiments, the method generates a recursive barcoding platform. For example, in embodiments wherein the first DNA element and/or second DNA element is a DNA barcode or plurality of DNA barcodes, the method can be used for tracking of cell lineages.

For example, the first DNA element and/or DNA element may be a gRNA or a plurality of gRNAs. Thus, in embodiments, the method includes generation of combinatorial gRNA libraries.

In embodiments, the first endonuclease targets the first endonuclease target site. In embodiments, the first endonuclease targets the third endonuclease target site. In embodiments, the first endonuclease targets the fourth endonuclease target site. In embodiments, the second endonuclease targets the second endonuclease target site. In embodiments, the second endonuclease targets the fifth endonuclease target site. In embodiments, the second endonuclease targets the sixth endonuclease target site.

In embodiments, the DNA element is a gene. In embodiments, the DNA element is a promoter. In embodiments, the DNA element is an enhancer. In embodiments, the DNA element is a terminator. In embodiments, the DNA element is an intron. In embodiments, the DNA element is an intergenic region. In embodiments, the DNA element is a barcode. In embodiments, the DNA element is a translation initiation site. In embodiments, the DNA element a gRNA. In embodiments, the DNA element is a fragment of any of the foregoing.

In embodiments, the recipient oligonucleotide is in a recipient plasmid. In embodiments, the recipient oligonucleotide is in the recipient cell genome.

In embodiments, the second donor plasmid further includes a seventh homologous recombination region and a seventh endonuclease target site between the components of (d) iii) and (d) iv). In embodiments, the first endonuclease targets the seventh endonuclease site.

In embodiments, the donor plasmid includes an oligonucleotide encoding a gRNA, and the recipient cell includes an oligonucleotide encoding an RNA-guided DNA endonuclease. In embodiments, the donor plasmid includes an oligonucleotide encoding a gRNA, and wherein the recipient cell genome includes an oligonucleotide encoding an RNA-guided DNA endonuclease. In embodiments, the donor plasmid includes an oligonucleotide encoding a gRNA, and the recipient plasmid includes an oligonucleotide encoding an RNA-guided DNA endonuclease. In embodiments, the donor plasmid includes an oligonucleotide encoding a gRNA, and a recipient helper plasmid includes an oligonucleotide encoding an RNA-guided DNA endonuclease. In embodiments, the donor plasmid includes an oligonucleotide encoding a gRNA, and the donor plasmid includes an oligonucleotide encoding an inducible RNA-guided DNA endonuclease. In embodiments, the donor plasmid includes an oligonucleotide encoding a gRNA, and the recipient genome includes an oligonucleotide encoding an inducible RNA-guided DNA endonuclease. In embodiments, the donor plasmid includes an oligonucleotide encoding a gRNA, and the recipient plasmid includes an oligonucleotide encoding an inducible RNA-guided DNA endonuclease. In embodiments, the donor plasmid includes an oligonucleotide encoding a gRNA, and a recipient helper plasmid includes an oligonucleotide encoding an inducible RNA-guided DNA endonuclease.

In embodiments, the recipient cell includes an inducible gRNA. In embodiments, the donor cell includes an oligonucleotide encoding an RNA-guided DNA endonuclease. In embodiments, the recipient cell includes an oligonucleotide encoding an RNA-guided DNA endonuclease. In embodiments, expression of the RNA-guided DNA is constitutive. In embodiments, expression of the RNA-guided DNA is inducible.

In embodiments, the RNA-guided DNA endonuclease is Cas9. In embodiments, the RNA-guided DNA endonuclease is Cas10. In embodiments, the RNA-guided DNA endonuclease is Cpf1. In embodiments, the RNA-guided DNA endonuclease is C2c1. In embodiments, the RNA-guided DNA endonuclease is C2c2. In embodiments, the RNA-guided DNA endonuclease is C2c3. In embodiments, the RNA-guided DNA endonuclease is Cas12c1. In embodiments, the RNA-guided DNA endonuclease is Cas12a. In embodiments, the RNA-guided DNA endonuclease is Cas12b. In embodiments, the RNA-guided DNA endonuclease is Cas12c2. In embodiments, the RNA-guided DNA endonuclease is Cas12g. In embodiments, the RNA-guided DNA endonuclease is Cas12e. In embodiments, the RNA-guided DNA endonuclease is Cas12i1. In embodiments, the RNA-guided DNA endonuclease is Cas12i2.

For the methods provided herein, in embodiments, an oligonucleotide encoding the first endonuclease is in the donor plasmid. In embodiments, an oligonucleotide encoding the first endonuclease is the recipient oligonucleotide. In embodiments, an oligonucleotide encoding the first endonuclease is in a recipient cell helper plasmid. In embodiments, an oligonucleotide encoding the first endonuclease is in the recipient genome. In embodiments, expression of the first endonuclease is inducible. In embodiments, the oligonucleotide encoding the second endonuclease is in the donor plasmid. In embodiments, an oligonucleotide encoding the second endonuclease is in the recipient oligonucleotide. In embodiments, an oligonucleotide encoding the second endonuclease is in a recipient cell helper plasmid.

In embodiments, the first endonuclease and/or the second endonuclease is a homing endonuclease. In embodiments, the homing endonuclease is I-ScaI, PI-SceI, I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-SceI, I-SceII, I-SceIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In embodiments, the homing endonuclease is I-ScaI. In embodiments, the homing endonuclease is PI-SceI. In embodiments, the homing endonuclease is I-AniI. In embodiments, the homing endonuclease is I-CeuI. In embodiments, the homing endonuclease is I-ChuI. In embodiments, the homing endonuclease is I-CpaI. In embodiments, the homing endonuclease is I-CpaII. In embodiments, the homing endonuclease is I-CreI. In embodiments, the homing endonuclease is I-DmoI. In embodiments, the homing endonuclease is H-DreI. In embodiments, the homing endonuclease is I-HmuI. In embodiments, the homing endonuclease is I-HmuII. In embodiments, the homing endonuclease is I-LlaI. In embodiments, the homing endonuclease is I-MsoI. In embodiments, the homing endonuclease is PI-PfuI. In embodiments, the homing endonuclease is PI-PkoII. In embodiments, the homing endonuclease is I-PorI. In embodiments, the homing endonuclease is I-PpoI. In embodiments, the homing endonuclease is PI-PspI. In embodiments, the homing endonuclease is I-SceI. In embodiments, the homing endonuclease is I-SceII. In embodiments, the homing endonuclease is I-SceIII. In embodiments, the homing endonuclease is I-SceIV. In embodiments, the homing endonuclease is I-SceV. In embodiments, the homing endonuclease is I-SceVI. In embodiments, the homing endonuclease is I-SceVII. In embodiments, the homing endonuclease is I-Ssp6803I. In embodiments, the homing endonuclease is I-TevI. In embodiments, the homing endonuclease is I-TevII. In embodiments, the homing endonuclease is I-TevIII. In embodiments, the homing endonuclease is PI-TliI. In embodiments, the homing endonuclease is PI-TliII. In embodiments, the homing endonuclease is I-TspO61I, or I-Vdi141I. In embodiments, the homing endonuclease is I-Vdi141I.

In embodiments, the endonuclease is a transcription activator-like effector nuclease. In embodiments, the endonuclease is a zinc finger nuclease.

For the methods provided herein, in embodiments, steps (d) to (e) are repeated for one or more iterations, thereby forming one or more subsequent assembled DNA elements. In embodiments, the first, second or subsequent donor plasmid includes a selectable marker selecting for integration of the first oligonucleotide, second oligonucleotide or subsequent oligonucleotide into the recipient cell oligonucleotide. In embodiments, the first donor plasmid includes a selectable marker selecting for integration of the first oligonucleotide into the recipient cell oligonucleotide. In embodiments, the second donor plasmid includes a selectable marker selecting for integration of the second oligonucleotide into the recipient cell oligonucleotide. In embodiments, the subsequent donor plasmid includes a selectable marker selecting for integration of the subsequent oligonucleotide into the recipient cell oligonucleotide

In embodiments, the first donor plasmid includes a selectable marker selecting for integration of the first oligonucleotide into the recipient oligonucleotide. In embodiments, the selectable marker is between the components of (a)(v) and (a)(iv). In embodiments, the second donor plasmid includes a selectable marker selecting for integration of the second oligonucleotide into the recipient oligonucleotide.

In embodiments, the recipient cell oligonucleotide includes a counter-selectable marker selecting for integration of the first oligonucleotide into the recipient cell oligonucleotide. In embodiments, the recombined recipient cell oligonucleotide includes a counter-selectable marker selecting for integration of the second or subsequent oligonucleotide into the recombined recipient cell oligonucleotide. Counter-selectable markers for use in the methods described herein are described above.

In embodiments, the assembled DNA element, which may also be referred to as a DNA assembly, is sequenced. In embodiments, the recipient oligonucleotide is sequenced. In embodiments, the recombinant recipient oligonucleotide is sequenced. In embodiments, the recombinant recipient oligonucleotide is a plasmid, wherein the plasmid is linearized, ligated to sequencing adaptors and sequenced. In embodiments, the assembled DNA element is amplified by PCR and sequenced. In embodiments, (a) the recipient cells are lysed, (b) the oligonucleotides are digested with an endonuclease or a plurality of endonucleases, (c) the assembled DNA element is isolated, and (d) the assembled DNA element or plurality of assembled genes are ligated to sequencing adaptors and sequenced. In embodiments, the assembled DNA element is isolated. In embodiments, the recombinant recipient oligonucleotide is isolated.

In embodiments, the assembled DNA element is from about 100 nucleotides to about 500,000 nucleotides in length. The length may be any value or subrange within the indicated ranges, including endpoints.

In embodiments, the assembled DNA element is from about 100 nucleotides, about 1000 nucleotides, about 10,000 nucleotides, about 20,000 nucleotides, about 40,000 nucleotides, about 60,000 nucleotides, about 80,000 nucleotides, about 100,000 nucleotides, about 120,000 nucleotides, about 140,000 nucleotides, about 160,000 nucleotides, about 180,000 nucleotides, about 20,000 nucleotides, about 240,000 nucleotides, about 260,000 nucleotides, about 280,000 nucleotides, about 300,000 nucleotides, about 320,000 nucleotides, about 340,000 nucleotides, about 360,000 nucleotides, about 380,000 nucleotides, about 400,000 nucleotides, about 420,000 nucleotides, about 440,000 nucleotides, about 460,000 nucleotides, about 480,000 nucleotides, or about 500,000 nucleotides in length. The length may be any value or subrange within the indicated ranges, including endpoints.

In embodiments, the first, second or subsequent homology regions and the corresponding first, second or subsequent homology regions are about 20 base pairs to about 500 base pairs in length. The length may be any value or subrange within the indicated ranges, including endpoints.

In embodiments, the first, second or subsequent homology regions and the corresponding first, second or subsequent homology regions are about 20 base pairs, 40 base pairs, 60 base pairs, 80 base pairs, 100 base pairs, 120 base pairs, 140 base pairs, 160 base pairs, 180 base pairs, 200 base pairs, 220 base pairs, 240 base pairs, 260 base pairs, 280 base pairs, 300 base pairs, 320 base pairs, 340 base pairs, 360 base pairs, 380 base pairs, 400 base pairs, 420 base pairs, 440 base pairs, 460 base pairs, 480 base pairs or 500 base pairs in length. In embodiments, the first, second or subsequent homology regions and the corresponding the first, second or subsequent homology regions are about 50 base pairs in length. The length may be any value or subrange within the indicated ranges, including endpoints.

### III. Methods of Analysis

In an aspect is provided a method of identifying an oligonucleotide from a mixture of oligonucleotides. The method includes: (a) providing a mixture of oligonucleotides, (b) inserting each oligonucleotide into a donor plasmid, wherein each donor plasmid includes, in sequential order: i) a first endonuclease cut site, ii) a first homologous recombination region, iii) a second homologous recombination region, and iv) a second endonuclease cut site, wherein the oligonucleotide is inserted between the first homologous recombination region and the second homologous recombination region, thereby producing a plurality of donor plasmids, each donor plasmid including a single oligonucleotide from the mixture of oligonucleotides; (c) transforming a plurality of host cells with the plurality of donor plasmids such that each host cell includes a donor plasmid, thereby forming a plurality of transformed host cells; (d) plating and culturing the plurality of transformed host cells on a first ordered array, wherein each transformed host cell produces a colony of clones in the first ordered array; (e) providing a plurality of recipient cells in a second ordered array, wherein each recipient cell includes a recipient oligonucleotide including, in sequential order: (i) a unique barcode sequence, wherein the unique barcode sequence identifies a position of the recipient cell in the second ordered array, (ii) a corresponding first homologous recombination region, wherein the first homologous recombination region is homologous to the corresponding first homologous recombination region, (iii) a third endonuclease cut site, and (iv) a corresponding second homologous recombination site, wherein the second homologous recombination region is homologous to the corresponding second homologous recombination region, wherein the first endonuclease cut site, the second endonuclease cut site, and the third endonuclease cut site can be cleaved by an endonuclease; (f) contacting each colony of clones from the first ordered array with a recipient cell in a corresponding site of the second ordered array under conditions that (i) transfer the donor plasmid from the colony of clones to the recipient cell by bacterial conjugation, (ii) cleave the first, second, and third endonuclease cut sites by the endonuclease, and (ii) transfer the oligonucleotide from the donor plasmid to the recipient cell oligonucleotide by homologous recombination, thereby producing a fusion sequence including the barcode sequence and the oligonucleotide; (g) sequencing the fusion sequence; and identifying the sequenced oligonucleotide in the first and/or second ordered array of donor cells and/or of recipient cells by identification of the barcode sequence.

In embodiments, plating and culturing the cells include plating and culturing the cells on a surface. For example, the surface may be a solid medium. Thus, in embodiments, a colony of clones is a colony of cells on a solid medium. In embodiments, plating and culturing the cells includes plating and culturing the cells in a liquid medium. For example, a single cell can be plated and cultured in a liquid medium. Thus, in embodiments, a colony of clones is a colony of cells in a liquid medium.

In embodiments, the recipient oligonucleotide is in a recipient cell plasmid. In embodiments, the recipient oligonucleotide is in the recipient cell genome. In embodiments, the donor plasmid includes a selectable marker between the first homologous recombination region and the second homologous recombination region selecting for integration of the oligonucleotide into the recipient cell oligonucleotide. In embodiments, the recipient cell oligonucleotide includes two endonuclease cut sites in step (e)(iii). In embodiments, the method further includes a counter-selectable marker between the two endonuclease cut sites, wherein the counter-selectable marker selects for integration of the oligonucleotide into the recipient oligonucleotide.

In an aspect is provided a method of identifying an oligonucleotide from a mixture of oligonucleotides. The method includes: (a) providing a plurality of host cells in a first ordered array, wherein each host cell includes a donor plasmid, wherein each donor plasmid includes, in sequential order: i) a first endonuclease cut site, ii) a first homologous recombination region, iii) a unique barcode sequence, iv) a second homologous recombination region, and v) a second endonuclease cut site; wherein the unique barcode sequence identifies a position of the host cell in the first ordered array; (b) providing a plurality of recipient cells, wherein each recipient cell includes a recipient oligonucleotide including an oligonucleotide from the plurality of oligonucleotides, wherein each recipient plasmid includes, in sequential order: i) the oligonucleotide sequence, ii) a corresponding first homologous recombination region, wherein the first homologous recombination region is homologous to the corresponding first homologous recombination region, iii) a third endonuclease cut site, wherein the first endonuclease cut site, the second endonuclease cut site, and the third endonuclease cut site can be cleaved by an endonuclease, and iv) a corresponding second homologous recombination region, wherein the second homologous recombination region is homologous to the corresponding second homologous recombination region; (c) plating and culturing the plurality of recipient cells on a second ordered array, wherein each recipient cell produces a colony of clones in the second ordered array; (d) contacting a donor cell from the first ordered array with each colony of clones in a corresponding site of the second ordered array under conditions that (i) transfer the donor plasmid from a donor cell to the colony of clones by bacterial conjugation, (ii) cleave the first, second, and third endonuclease cut sites by the endonuclease, and (iii) transfer the barcode sequence from the donor plasmid to the recipient cell oligonucleotide by homologous recombination, thereby producing a fusion sequence including the barcode sequence and the oligonucleotide; (e) sequencing the fusion sequence; and (f) identifying the sequenced oligonucleotide in the first and/or second ordered array of recipient cells by identification of the barcode sequence.

In embodiments, the recipient oligonucleotide is in a recipient cell plasmid.

In embodiments, the recipient oligonucleotide is in the recipient cell genome. In embodiments, the donor plasmid includes a selectable marker between the first homologous recombination region and the second homologous recombination region selecting for integration of the barcode into the recipient oligonucleotide. In embodiments, the recipient oligonucleotide includes two endonuclease cut sites in step (b)(iii). In embodiments, the method further includes a counter-selectable marker between the two endonuclease cut sites selecting for integration of the barcode into the recipient cell oligonucleotide.

For the methods provided herein, in embodiments, the first endonuclease cut site, the second endonuclease cut site, and the third endonuclease cut site are the same endonuclease cut site. In embodiments, the first endonuclease cut site, the second endonuclease cut site, and the third endonuclease cut site are different endonuclease cut sites. In embodiments, the endonuclease includes multiple endonucleases.

In embodiments, the endonuclease is encoded by an oligonucleotide in the recipient cell. In embodiments, the oligonucleotide is in the recipient cell genome. In embodiments, wherein the oligonucleotide is in the recipient plasmid. In embodiments, the oligonucleotide encoding an endonuclease is in a helper plasmid. In embodiments, the endonuclease is encoded by an oligonucleotide in the donor plasmid. In embodiments, the endonuclease is encoded by an oligonucleotide in the donor plasmid. In embodiments, expression of the endonuclease is inducible.

In embodiments, the endonuclease is a transcription activator-like effector nuclease. In embodiments, the endonuclease is a zinc finger nuclease. In embodiments, the endonuclease is HO.

In embodiments, the RNA-guided DNA endonuclease is a CRISPR system. In embodiments, the endonuclease is an RNA-guided DNA endonuclease. In embodiments, the RNA-guided DNA endonuclease is Cas9. In embodiments, the RNA-guided DNA endonuclease is Cas10. In embodiments, the RNA-guided DNA endonuclease is Cpf1. In embodiments, the RNA-guided DNA endonuclease is C2c1. In embodiments, the RNA-guided DNA endonuclease is C2c2. In embodiments, the RNA-guided DNA endonuclease is C2c3. In embodiments, the RNA-guided DNA endonuclease is Cas12c1. In embodiments, the RNA-guided DNA endonuclease is Cas12a. In embodiments, the RNA-guided DNA endonuclease is Cas12b. In embodiments, the RNA-guided DNA endonuclease is Cas12c2. In embodiments, the RNA-guided DNA endonuclease is Cas12g. In embodiments, the RNA-guided DNA endonuclease is Cas12e. In embodiments, the RNA-guided DNA endonuclease is Cas12i1. In embodiments, the RNA-guided DNA endonuclease is Cas12i2.

For the methods provided herein, in embodiments, the donor plasmid includes an oligonucleotide encoding a guide RNA, and the recipient genome includes an oligonucleotide encoding an RNA-guided DNA endonuclease. In embodiments, the donor plasmid includes an oligonucleotide encoding a guide RNA, and the recipient plasmid includes an oligonucleotide encoding an RNA-guided DNA endonuclease. In embodiments, the donor plasmid includes an oligonucleotide encoding a guide RNA, and the recipient helper plasmid includes an oligonucleotide encoding an RNA-guided DNA endonuclease. In embodiments, the donor plasmid includes an oligonucleotide encoding a guide RNA, and the recipient plasmid includes an oligonucleotide encoding an inducible RNA-guided DNA endonuclease.

In embodiments, the recipient cell includes an inducible gRNA. In embodiments, the donor cell includes an oligonucleotide encoding an RNA-guided DNA endonuclease. In embodiments, the recipient cell includes an oligonucleotide encoding an RNA-guided DNA endonuclease. In embodiments, expression of the RNA-guided DNA is constitutive. In embodiments, expression of the RNA-guided DNA is inducible.

In embodiments, the method further includes isolating the donor plasmid. In embodiments, the method further includes isolating the recipient plasmid. In embodiments, the method further includes isolating the recombinant recipient plasmid. In embodiments, the method further includes isolating the sequenced oligonucleotide. In embodiments, the method further includes isolating one or more of the donor cell, the recipient cell, the recombinant recipient cell, the recipient oligonucleotide, or the recombinant recipient oligonucleotide.

In embodiments, the method includes combining one or more subset of colonies. Thus, in embodiments, the method includes combining one or more subset of colonies and isolating a plurality of donor plasmids from the subset of colonies. In embodiments, the method includes combining one or more subset of colonies and isolating a plurality of recipient plasmids from the subset of colonies. In embodiments, the method includes combining one or more subset of colonies and isolating a plurality of recombinant recipient plasmids from the subset of colonies. In embodiments, the method further includes isolating a plurality of sequenced oligonucleotides.

In embodiments, the recipient cell or the donor cell includes an oligonucleotide that enables plasmid conjugation. In embodiments, the oligonucleotide that enables plasmid conjugation is in the donor cell genome. In embodiments, the oligonucleotide that enables plasmid conjugation is in a helper plasmid. In embodiments, the oligonucleotide that enables plasmid conjugation is the Tra operon. Other oligonucleotides that enable plasmid conjugation are described above.

In embodiments, donor cells, recipient cells, or recombinant recipient cells are transferred to positions on a third ordered array, a fourth ordered array, or a subsequent ordered array.

In embodiments, the barcode sequence is from about 4 nucleotides to about 50 nucleotides in length. In embodiments, the barcode sequence is from about 8 nucleotides to about 50 nucleotides in length. In embodiments, the barcode sequence is from about 12 nucleotides to about 50 nucleotides in length. In embodiments, the barcode sequence is from about 16 nucleotides to about 50 nucleotides in length. In embodiments, the barcode sequence is from about 20 nucleotides to about 50 nucleotides in length. In embodiments, the barcode sequence is from about 24 nucleotides to about 50 nucleotides in length. In embodiments, the barcode sequence is from about 28 nucleotides to about 50 nucleotides in length. In embodiments, the barcode sequence is from about 32 nucleotides to about 50 nucleotides in length. In embodiments, the barcode sequence is from about 36 nucleotides to about 50 nucleotides in length. The length of the barcode be any value or subrange within ranges provided herein, including endpoints.

In embodiments, the barcode sequence is from about 4 nucleotides to about 36 nucleotides in length. In embodiments, the barcode sequence is from about 4 nucleotides to about 32 nucleotides in length. In embodiments, the barcode sequence is from about 4 nucleotides to about 28 nucleotides in length. In embodiments, the barcode sequence is from about 4 nucleotides to about 24 nucleotides in length. In embodiments, the barcode sequence is from about 4 nucleotides to about 20 nucleotides in length. In embodiments, the barcode sequence is from about 4 nucleotides to about 16 nucleotides in length. In embodiments, the barcode sequence is from about 4 nucleotides to about 12 nucleotides in length. In embodiments, the barcode sequence is from about 4 nucleotides to about 8 nucleotides in length. In embodiments, the barcode sequence is about 4 nucleotides, 8 nucleotides, 12 nucleotides, 16 nucleotides, 20 nucleotides, 24 nucleotides, 28 nucleotides, 32 nucleotides, 36 nucleotides, or 40 nucleotides in length. In embodiments, the barcode sequence is about 15 nucleotides in length. In embodiments, the barcode sequence is about 40 nucleotides in length. The length of the barcode be any value or subrange within ranges provided herein, including endpoints.

### Further Embodiments

The invention is further described by the following additional embodiments.

Embodiment 1: A method of assembling a DNA element, the method comprising: (1) providing a first host cell comprising a first donor plasmid comprising, in sequential order: a first endonuclease target site, a first homologous recombination region, optionally a first oligonucleotide comprising a first DNA element fragment, a second homologous recombination region, a second endonuclease target site, and a third endonuclease target site; (2) providing a recipient cell, wherein the recipient cell comprises a recipient oligonucleotide comprising: a third homologous recombination region, wherein the third homologous region is homologous to the first homologous recombination region, a fourth endonuclease target site, and a fourth homologous region, wherein the fourth homologous recombination region is homologous to the second homologous recombination region; and (3) contacting the first host cell with the recipient cell under conditions to (i) transfer the first donor plasmid from the first host cell to the recipient cell by bacterial conjugation, (ii) direct a first endonuclease to the at least one of the first endonuclease target site, the third endonuclease target site, or the fourth endonuclease target site, thereby producing double-stranded breaks in the first donor plasmid and the recipient oligonucleotide and (iii) recombine the first donor plasmid and the recipient oligonucleotide in the recipient cell by homologous recombination via the first and second homologous recombination regions with the third and fourth corresponding homologous recombination regions, thereby forming a recombined recipient oligonucleotide; (4) providing a second host cell comprising a second donor plasmid comprising, in sequential order: a fifth endonuclease target site, a fifth homologous recombination region which is homologous to the second homologous region, a second oligonucleotide encoding a second DNA element fragment, a sixth homologous recombination region which is homologous to the fourth homologous region, and a sixth endonuclease target site; and (5) contacting the second host cell with the recipient cell containing the recombined recipient oligonucleotide under conditions to (i) transfer the second donor plasmid from the second host cell to the recipient cell by bacterial conjugation, (ii) express a second endonuclease, (iii) direct the second endonuclease to the second endonuclease target site, the fifth endonuclease target site and/or the sixth endonuclease target site, thereby producing double-stranded breaks, (iv) recombine the second donor plasmid and the recombined recipient oligonucleotide in the recipient cell by homologous recombination via the fifth and sixth homologous recombination sites with the corresponding second and fourth homologous recombination sites, thereby forming a second recombined recipient oligonucleotide comprising an assembled DNA element.

In further embodiments, step (a) comprises a plurality of first host cells, wherein each cell comprises a different first oligonucleotide and/or step (d) comprises a plurality of second host cells, wherein each cell comprises a different second oligonucleotide.

In further embodiments, each first host cell is in a position in a first ordered array.

In further embodiments, a plurality of first host cells are in a position in a first ordered array, thereby forming a pool of first host cells in the first ordered array.

In further embodiments, each second host cell is in a position in a second ordered array.

In further embodiments, a plurality of second host cells are in a position in a second ordered array, thereby forming a pool of second host cells in each position in the second ordered array.

In further embodiments, the first donor cell is in a first ordered array, the second donor cell is in a second ordered array, and one or more subsequent donor cells are in one or more subsequent arrays.

In further embodiments, the method generates a combinatorial library comprising a plurality of different assembled DNA elements.

In further embodiments, the first endonuclease targets the first, third, or fourth endonuclease target site.

In further embodiments, the second endonuclease targets the second, fifth, or sixth endonuclease target site.

In further embodiments, the DNA element is a gene, a promoter, an enhancer, a terminator, an intron, an intergenic region, a barcode, or a gRNA.

In further embodiments, the recipient oligonucleotide is in a recipient plasmid.

In further embodiments, the recipient oligonucleotide is in the recipient cell genome.

In further embodiments, the second donor plasmid further comprises a seventh homologous recombination region and a seventh endonuclease target site between the components of (d) iii) and (d) iv).

In further embodiments, the first endonuclease targets the seventh endonuclease site.

In further embodiments, the first and second endonucleases are independently selected from an RNA-guided DNA endonuclease, a homing endonuclease, a transcription activator-like effector nuclease, and a zinc finger nuclease.

In further embodiments, an oligonucleotide encoding the first endonuclease is in the donor cell or the recipient cell.

In further embodiments, expression of the first and/or second endonuclease is inducible.

In further embodiments, an oligonucleotide encoding the second endonuclease is in the donor cell or the recipient cell.

In further embodiments, steps (d) to (e) are repeated for one or more iterations thereby forming one or more subsequent assembled DNA elements.

In further embodiments, the first, second or subsequent donor plasmid comprises a selectable marker selecting for integration of the first oligonucleotide, second oligonucleotide or subsequent oligonucleotide into the recipient cell oligonucleotide.

In further embodiments, the first donor plasmid comprises a selectable marker selecting for integration of the first oligonucleotide into the recipient oligonucleotide, optionally wherein the selectable marker is between the second and third endonuclease target sites. In embodiments, the second donor plasmid comprises a selectable marker selecting for integration of the second oligonucleotide into the recipient oligonucleotide.

In further embodiments, the recipient cell oligonucleotide comprises a counter-selectable marker selecting for integration of the first oligonucleotide into the recipient cell oligonucleotide.

In further embodiments, the donor plasmid comprises an origin of transfer, optionally wherein the origin of transfer is from a mobile element.

In further embodiments, the donor plasmid comprises a conditional replication origin; optionally wherein the conditional replication origin is dependent on presence of an oligonucleotide or a condition of cell growth.

In further embodiments, the donor plasmid or recipient oligonucleotide comprises a replicon that can replicate plasmids of lengths greater than 30 kilobases.

In further embodiments, the donor plasmid or recipient oligonucleotide comprises an inducible high-copy replication of origin.

In further embodiments, the donor plasmid or recipient oligonucleotide is a yeast artificial chromosome (YAC), a mammalian artificial chromosome (MAC), a human artificial chromosome (HAC), or a plant artificial chromosome. In further embodiments, the donor plasmid or recipient oligonucleotide is a viral vector.

In further embodiments, the donor cell comprises an oligonucleotide that enables plasmid conjugation.

In further embodiments, the donor cell or the recipient cell comprises an oligonucleotide encoding one or more homologous DNA repair genes; optionally wherein homologous DNA repair gene expression is inducible.

In further embodiments, the donor cell or recipient cell comprises an oligonucleotide encoding one or more recombination-mediated genetic engineering genes.

In further embodiments, the donor cell and the recipient cell are independently a bacteria cell.

In further embodiments, the assembled DNA element is sequenced, the recipient oligonucleotide is sequenced, and/or the recombinant recipient oligonucleotide is sequenced.

In further embodiments, the recombinant recipient oligonucleotide is a plasmid, and the plasmid is linearized, ligated to sequencing adaptors and sequenced.

In further embodiments, the assembled DNA element is amplified by PCR and sequenced; optionally wherein (a) the recipient cells are lysed, (b) the oligonucleotides are digested with an endonuclease or a plurality of endonucleases, (c) the assembled DNA element is isolated, and (d) the assembled DNA element or plurality of assembled genes are ligated to sequencing adaptors and sequenced.

In further embodiments, the assembled DNA element or recombinant recipient oligonucleotide is isolated.

In further embodiments, the assembled DNA fragment is from 100 nucleotides to 500,000 nucleotides in length.

In further embodiments, the first, second or subsequent homology regions and the corresponding first, second or subsequent homology regions are about 20 base pairs to about 500 base pairs in length.

In further embodiments, the first, second or subsequent homology regions and the corresponding the first, second or subsequent homology regions are about 50 base pairs in length.

In embodiments, provided herein is a method of identifying an oligonucleotide from a mixture of oligonucleotides, the method comprising: (a) providing a mixture of oligonucleotides, (b) inserting each oligonucleotide into a donor plasmid, wherein each donor plasmid comprises, in sequential order: (i) a first endonuclease cut site, (ii) a first homologous recombination region, (iii) a second homologous recombination region, and (iv) a second endonuclease cut site, wherein the oligonucleotide is inserted between the first homologous recombination region and the second homologous recombination region, thereby producing a plurality of donor plasmids, each donor plasmid comprising a single oligonucleotide from the mixture of oligonucleotides; (c) transforming a plurality of host cells with the plurality of donor plasmids such that each host cell comprises a donor plasmid, thereby forming a plurality of transformed host cells; (d) plating and culturing the plurality of transformed host cells on a first ordered array, wherein each transformed host cell produces a colony of clones in the first ordered array; (e) providing a plurality of recipient cells in a second ordered array, wherein each recipient cell comprises a recipient oligonucleotide comprising, in sequential order: (i) a unique barcode sequence, wherein the unique barcode sequence identifies a position of the recipient cell in the second ordered array, (ii) a corresponding first homologous recombination region, wherein the first homologous recombination region is homologous to the corresponding first homologous recombination region, (iii) a third endonuclease cut site, and (iv) a corresponding second homologous recombination site, wherein the second homologous recombination region is homologous to the corresponding second homologous recombination region, wherein the first endonuclease cut site, the second endonuclease cut site, and the third endonuclease cut site can be cleaved by an endonuclease; (f) contacting each colony of clones from the first ordered array with a recipient cell in a corresponding site of the second ordered array under conditions that (i) transfer the donor plasmid from the colony of clones to the recipient cell by bacterial conjugation, (ii) cleave the first, second, and third endonuclease cut sites by the endonuclease, and (ii) transfer the oligonucleotide from the donor plasmid to the recipient cell oligonucleotide by homologous recombination, thereby producing a fusion sequence comprising the barcode sequence and the oligonucleotide; (g) sequencing the fusion sequence; and (h) identifying the sequenced oligonucleotide in the first and/or second ordered array of donor cells and/or of recipient cells by identification of the barcode sequence.

In further embodiments, the recipient oligonucleotide is in a recipient cell plasmid.

In further embodiments, the recipient oligonucleotide is in the recipient cell genome.

In further embodiments, the methods provide that the donor plasmid comprises a selectable marker between the first homologous recombination region and the second homologous recombination region selecting for integration of the oligonucleotide into the recipient cell oligonucleotide.

In further embodiments, the methods herein provide that the recipient cell oligonucleotide comprises two endonuclease cut sites in step (e)(iii).

In embodiments herein, the method further comprises a counter-selectable marker between the two endonuclease cut sites, wherein the counter-selectable marker selects for integration of the oligonucleotide into the recipient oligonucleotide.

In embodiments, provided herein is a method of identifying an oligonucleotide from a plurality of oligonucleotides, the method comprising: (a) providing a plurality of host cells in a first ordered array, wherein each host cell comprises a donor plasmid, wherein each donor plasmid comprises, in sequential order: (i) a first endonuclease cut site, (ii) a first homologous recombination region, (iii) a unique barcode sequence, (iv) a second homologous recombination region, and (v) a second endonuclease cut site; wherein the unique barcode sequence identifies a position of the host cell in the first ordered array; (b) providing a plurality of recipient cells, wherein each recipient cell comprises a recipient oligonucleotide comprising an oligonucleotide from the plurality of oligonucleotides, wherein each recipient plasmid comprises, in sequential order: i) the oligonucleotide sequence, ii) a corresponding first homologous recombination region, wherein the first homologous recombination region is homologous to the corresponding first homologous recombination region, iii) a third endonuclease cut site, wherein the first endonuclease cut site, the second endonuclease cut site, and the third endonuclease cut site can be cleaved by an endonuclease, and iv) corresponding second homologous recombination region, wherein the second homologous recombination region is homologous to the corresponding second homologous recombination region; (c) plating and culturing the plurality of recipient cells on a second ordered array, wherein each recipient cell produces a colony of clones in the second ordered array; (d) contacting a donor cell from the first ordered array with each colony of clones in a corresponding site of the second ordered array under conditions that (i) transfer the donor plasmid from a donor cell to the colony of clones by bacterial conjugation, (ii) cleave the first, second, and third endonuclease cut sites by the endonuclease, and (iii) transfer the barcode sequence from the donor plasmid to the recipient cell oligonucleotide by homologous recombination, thereby producing a fusion sequence comprising the barcode sequence and the oligonucleotide; (e) sequencing the fusion sequence; and (f) identifying the sequenced oligonucleotide in the first and/or second ordered array of donor cells and/or of recipient cells by identification of the barcode sequence.

In embodiments, the method provides that the recipient oligonucleotide is in a recipient cell plasmid.

In further embodiments, the recipient oligonucleotide is in the recipient cell genome.

In embodiments, the donor plasmid comprises a selectable marker between the first homologous recombination region and the second homologous recombination region selecting for integration of the barcode into the recipient oligonucleotide.

In further embodiments, the recipient oligonucleotide comprises two endonuclease cut sites in step (b)(iii).

In further embodiments, the methods comprise provide a counter-selectable marker between the two endonuclease cut sites selecting for integration of the barcode into the recipient cell oligonucleotide.

In further embodiments, the first endonuclease cut site, the second endonuclease cut site, and the third endonuclease cut site are the same endonuclease cut site.

In further embodiments, the first endonuclease cut site, the second endonuclease cut site, and the third endonuclease cut site are different endonuclease cut sites.

In further embodiments, the endonuclease comprises multiple endonucleases.

In further embodiments, the donor plasmid comprises an origin of transfer.

In further embodiments, the origin of transfer is from a mobile element.

In further embodiments, the donor plasmid comprises a conditional replication origin.

In further embodiments, the conditional replication origin depends on the presence of an oligonucleotide.

In further embodiments, the conditional replication origin depends on a condition of cell growth.

In further embodiments, the donor plasmid or recipient plasmid comprises a replicon that can replicate plasmids at least 30 kilobases in length.

In further embodiments, the replicon is from a P1-derived artificial chromosome or a bacterial artificial chromosome.

In further embodiments, the donor plasmid or recipient cell oligonucleotide comprises an inducible high-copy replication of origin.

In further embodiments, the donor plasmid or recipient cell oligonucleotide is a yeast artificial chromosome (YAC), a mammalian artificial chromosome (MAC), a human artificial chromosome (HAC), or a plant artificial chromosome.

In further embodiments, the donor plasmid or recipient oligonucleotide is a viral vector.

In further embodiments, the endonuclease is encoded by an oligonucleotide in the recipient cell.

In further embodiments, the endonuclease is encoded by an oligonucleotide in the donor plasmid.

In further embodiments, the endonuclease is a homing endonuclease.

In further embodiments, the endonuclease is an RNA-guided DNA endonuclease.

In further embodiments, the endonuclease is HO.

In further embodiments, the methods further comprise isolating the donor plasmid.

In further embodiments, the methods further comprise isolating the recipient plasmid.

In further embodiments, the methods further comprise isolating the recombinant recipient plasmid.

In further embodiments, the methods further comprise isolating the sequenced oligonucleotide.

In further embodiments, the donor cell or recipient cell comprises an oligonucleotide that enables plasmid conjugation.

In further embodiments, the donor cell or recipient cell comprises an oligonucleotide encoding one or more homologous DNA repair genes.

In further embodiments, the donor cell or recipient cell comprises an oligonucleotide encoding one or more recombination-mediated genetic engineering genes.

In further embodiments, the donor cells, recipient cells, or recombinant recipient cells are transferred to positions on a third ordered array, a fourth ordered array, or a subsequent ordered array.

In further embodiments, the donor cell and the recipient cells are independently bacteria cells.

In further embodiments, the barcode sequence is from about 4 nucleotides to about 40 nucleotides in length.

In further embodiments, the barcode sequence is about 15 nucleotides in length.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Methods for an Vivo DNA Stitching

Bacterial strains: BUN20 [Δlac-169 rpoS(Am) robA1 creC510 hsdR514 ΔuidA(MluI):pir-116 endA(BT333) recA1 F′(lac+pro+ΔoriT:tet)] was used as the donor strain (Li, M. et al. Nat. Genet. 37, 311-319 (2005)). BW23474: [Alac-169 rpoS(Am) robA1 creC510 hsdR514 ΔuidA(MluI):pir-116 endA(BT333) recA1] was used as the host strain for cloning and propagation of all donor plasmids (Haldimann, A. et al. Proc. Natl. Acad. Sci. 93, 14361 (1996)). BW28705 [lacIQ rrnB3 ΔlacZ4787 hsdR514 Δ (araBAD)567 Δ (rhaBAD)568 galU95 ΔendA9:FRT ΔrecA635:FRT] or RE1133 (Egbert et al., Nucleic Acids Research, vol. 47 (6), 8 Apr. 2019, Pages 3244-3256) [cmR::mutS pTet2-gam-bet-exo-dam/tetR::bioA/B ilvG+dnaG.Q576A lacIQ1 Pcp8-araE ΔaraBAD pConst-araC ΔrecJ ΔxonA Pkm-cymR-Cas9::bioC] were used as the recipient strains for in vivo stitching. DH5α and DH10β were used for cloning recipient plasmids.

DNA oligonucleotides used for the first and second steps PCR are given in Tables 1 and 2.

TABLE 1
Primers for the first step PCR

| | | |
|---|---|---|
| pBPS_fwr_1 | ACACTCTTTCCCTACACGACGCTCTTCCGATCTNNNNNNNNCGATGT | SEQ ID NO: 1 |
| | ttcggttagagcggatgtg | |
| pBPS_fwr_2 | ACACTCTTTCCCTACACGACGCTCTTCCGATCTNNNNNNNNACAGTG | SEQ ID NO: 2 |
| | ttcggttagagcggatgtg | |
| pBPS_fwr_3 | ACACTCTTTCCCTACACGACGCTCTTCCGATCTNNNNNNNNTGACCA | SEQ ID NO: 3 |
| | ttcggttagagcggatgtg | |
| pBPS_fwr_4 | ACACTCTTTCCCTACACGACGCTCTTCCGATCTNNNNNNNNGCCAAT | SEQ ID NO: 4 |
| | ttcggttagagcggatgtg | |
| pBPS_fwr_5 | ACACTCTTTCCCTACACGACGCTCTTCCGATCTNNNNNNNNATCACG | SEQ ID NO: 5 |
| | ttcggttagagcggatgtg | |
| pBPS_fwr_6 | ACACTCTTTCCCTACACGACGCTCTTCCGATCTNNNNNNNNGGCTAC | SEQ ID NO: 6 |
| | ttcggttagagcggatgtg | |
| pBPS_fwr_7 | ACACTCTTTCCCTACACGACGCTCTTCCGATCTNNNNNNNNTAGCTT | SEQ ID NO: 7 |
| | ttcggttagagcggatgtg | |
| pBPS_fwr_8 | ACACTCTTTCCCTACACGACGCTCTTCCGATCTNNNNNNNNTCTCCC | SEQ ID NO: 8 |
| | ttcggttagagcggatgtg | |
| pBPS_rev_1 | GTGACTGGAGTTCAGACGTGTGCTCTTCCGATCNNNNNNNNTATATA | SEQ ID NO: 9 |
| | CGCaggtaacccatatgcatggc | |
| pBPS_rev_2 | GTGACTGGAGTTCAGACGTGTGCTCTTCCGATCNNNNNNNNCGCTCT | SEQ ID NO: 10 |
| | ATCaggtaacccatatgcatggc | |
| pBPS_rev_3 | GTGACTGGAGTTCAGACGTGTGCTCTTCCGATCNNNNNNNNGAGACG | SEQ ID NO: 11 |
| | TCTaggtaacccatatgcatggc | |
| pBPS_rev_4 | GTGACTGGAGTTCAGACGTGTGCTCTTCCGATCNNNNNNNNATACTG | SEQ ID NO: 12 |
| | CGTaggtaacccatatgcatggc | |
| pBPS_rev_5 | GTGACTGGAGTTCAGACGTGTGCTCTTCCGATCNNNNNNNNACTAGC | SEQ ID NO: 13 |
| | AGAaggtaacccatatgcatggc | |
| pBPS_rev_6 | GTGACTGGAGTTCAGACGTGTGCTCTTCCGATCNNNNNNNNCTGCTA | SEQ ID NO: 14 |
| | CTCaggtaacccatatgcatggc | |
| pBPS_rev_7 | GTGACTGGAGTTCAGACGTGTGCTCTTCCGATCNNNNNNNNCCGTAC | SEQ ID NO: 15 |
| | ACAaggtaacccatatgcatggc | |
| pBPS_rev_8 | GTGACTGGAGTTCAGACGTGTGCTCTTCCGATCNNNNNNNNATCTGC | SEQ ID NO: 16 |
| | AAaggtaacccatatgcatggc | |

TABLE 2
Primers for the second step PCR

| | | |
|---|---|---|
| D501 | AATGATACGGCGACCACCGAGATCTACACTATAGCCTACACTCTTTCCCTA | SEQ ID NO: 17 |
| | CACGACGCTCTTCCGATCT | |
| D502 | AATGATACGGCGACCACCGAGATCTACACATAGAGGCACACTCTTTCCCTA | SEQ ID NO: 18 |
| | CACGACGCTCTTCCGATCT | |
| D503 | AATGATACGGCGACCACCGAGATCTACACCCTATCCTACACTCTTTCCCTA | SEQ ID NO: 19 |
| | CACGACGCTCTTCCGATCT | |
| D504 | AATGATACGGCGACCACCGAGATCTACACGGCTCTGAACACTCTTTCCCTA | SEQ ID NO: 20 |
| | CACGACGCTCTTCCGATCT | |
| D505 | AATGATACGGCGACCACCGAGATCTACACAGGCGAAGACACTCTTTCCCTA | SEQ ID NO: 21 |
| | CACGACGCTCTTCCGATCT | |
| D506 | AATGATACGGCGACCACCGAGATCTACACTAATCTTAACACTCTTTCCCTA | SEQ ID NO: 22 |
| | CACGACGCTCTTCCGATCT | |
| D507 | AATGATACGGCGACCACCGAGATCTACACCAGGACGTACACTCTTTCCCTA | SEQ ID NO: 23 |
| | CACGACGCTCTTCCGATCT | |
| D508 | AATGATACGGCGACCACCGAGATCTACACGTACTGACACACTCTTTCCCTA | SEQ ID NO: 24 |
| | CACGACGCTCTTCCGATCT | |
| D701 | CAAGCAGAAGACGGCATACGAGATCGAGTAATGTGACTGGAGTTCAGACGT | SEQ ID NO: 25 |
| | GTGCTCTTCCGATC | |
| D702 | CAAGCAGAAGACGGCATACGAGATTCTCCGGAGTGACTGGAGTTCAGACGT | SEQ ID NO: 26 |
| | GTGCTCTTCCGATC | |
| D703 | CAAGCAGAAGACGGCATACGAGATAATGAGCGGTGACTGGAGTTCAGACGT | SEQ ID NO: 27 |
| | GTGCTCTTCCGATC | |
| D704 | CAAGCAGAAGACGGCATACGAGATGGAATCTCGTGACTGGAGTTCAGACGT | SEQ ID NO: 28 |
| | GTGCTCTTCCGATC | |
| D705 | CAAGCAGAAGACGGCATACGAGATTTCTGAATGTGACTGGAGTTCAGACGT | SEQ ID NO: 29 |
| | GTGCTCTTCCGATC | |
| D706 | CAAGCAGAAGACGGCATACGAGATACGAATTCGTGACTGGAGTTCAGACGT | SEQ ID NO: 30 |
| | GTGCTCTTCCGATC | |
| D707 | CAAGCAGAAGACGGCATACGAGATAGCTTCAGGTGACTGGAGTTCAGACGT | SEQ ID NO: 31 |
| | GTGCTCTTCCGATC | |
| D708 | CAAGCAGAAGACGGCATACGAGATGCGCATTAGTGACTGGAGTTCAGACGT | SEQ ID NO: 32 |
| | GTGCTCTTCCGATC | |
| D709 | CAAGCAGAAGACGGCATACGAGATCATAGCCGGTGACTGGAGTTCAGACGT | SEQ ID NO: 33 |
| | GTGCTCTTCCGATC | |
| D710 | CAAGCAGAAGACGGCATACGAGATTTCGCGGAGTGACTGGAGTTCAGACGT | SEQ ID NO: 34 |
| | GTGCTCTTCCGATC | |
| D711 | CAAGCAGAAGACGGCATACGAGATGCGCGAGAGTGACTGGAGTTCAGACGT | SEQ ID NO: 35 |
| | GTGCTCTTCCGATC | |
| D712 | CAAGCAGAAGACGGCATACGAGATCTATCGCTGTGACTGGAGTTCAGACGT | SEQ ID NO: 36 |
| | GTGCTCTTCCGATC | |

Media and chemicals: Luria-Bertani (LB) broth (1% w/v tryptone, 0.5% w/v yeast extract, 1% w/v NaCl) as complex medium was routinely used for cloning and for growth of donor and recipient plasmids. To maintain plasmids, antibiotics were added at concentrations listed in Table 3. For LB media including hygromycin, 0.5% w/v sodium chloride was used because hygromycin is salt sensitive. L-arabinose (0.2% w/v), L-rhamnose (0.2% w/v) anhydrotetracycline (100 ng/ml), 4-Isopropylbenzoic acid (cumate; 15 ug/ml), and isopropyl β-d-1-thiogalactopyranoside (IPTG; 500 uM) were used to induce the P_{araBAD}, P_{rhaBAD}, P_{Tet2}, Pₖₘ-cymR, and P_{lacIQ} promoters, respectively. Sucrose agar plates (0.5% w/v yeast extract, 1% w/v tryptone, 6% w/v sucrose, 1.5% agar) and appropriate amounts of antibiotics were used to select against the SacB counter-selectable marker. C1-Phe agar plates (0.5% w/v yeast extract, 1% w/v NaCl, 0.4% w/v glycerol, 2% w/v agar, 10 mM D, L-p-C1-Phe) and the appropriate amounts of antibiotics were used for the counterselection of PheS Gly²⁹⁴. YEG agar plates (0.5% w/v yeast extract, 1% w/v NaCl, 0.4% w/v glucose, 2% w/v agar) and the appropriate amounts of antibiotics were used for subcloning recipient plasmids that contain the PheS Gly²⁹⁴ fragment.

TABLE 3
Selection drug concentrations

| Drug | Working concentration | |
|---|---|---|
| Hygromycin B | 200 | ug/ml |
| Nourseothricin Sulfate | 100 | ug/ml |
| Kanamycin | 50 | ug/ml |
| Gentamicin | 20 | ug/ml |
| Spectinomycin | 50 | ug/ml |
| D,L-p-Cl-Phe | 200 | ug/ml |
| Zeocin | 5 | ug/ml |
| Chloramphenicol | 25 | ug/ml |
| Ampicillin | 50 | ug/ml |

Plasmid sequences used for in vivo DNA stitching can be found in Table 4.

TABLE 4
Plasmids used in in vivo stitching.

| | | Homologous | Swapping | |
|---|---|---|---|---|
| Name | Plasmid Type | Regions | cassette | Other features |
| pSL270 | Helper | NA | NA | P_{rhaBAD}-red |
| pSL359 | Helper | NA | NA | P_{rhaBAD}-red, P_{araBAD}-Cas9 |
| pML300 | Helper | NA | NA | P_{rhaBAD}-red |
| pSL402 | Recipient | H1, H3 | PheS-ampR | CmR |
| pSL414 | Donor | H1, H3 | GmR-SacB | oriT, kanR, P_{J23119}-gRNA^{T1} |
| pSL415 | Donor | H1, H3 | GmR-SacB | oriT, kanR, P_{J23119}-mock |
| pSL398 | Recipient | H1, H3 | ZeoR-SacB | GmR |
| pSL485 | Donor | H1, H3 | PheS-ampR | oriT, kanR, P_{J23119}-gRNA^{T1}, |
| | | | | mEGFP¹⁻²⁵¹ |
| pSL486 | Donor | H3 | HygR-SacB | oriT, kanR, P_{J23119}-gRNA^{T3}, |
| | | | | mEGFP¹⁹⁸⁻⁵¹⁷ |
| pSL488 | Donor | H3 | PheS-ampR | oriT, kanR, P_{J23119}-gRNA^{T1}, |
| | | | | mEGFP⁴⁵⁴⁻⁷²⁰ |
| pSL684 | Donor | H3 | PheS-ampR | oriT, kanR, P_{J23119}-gRNA^{T1}, |
| | | | | mEGFP⁵²⁰⁻⁷²⁰ |
| pSL685 | Donor | H3 | PheS-ampR | oriT, kanR, P_{J23119}-gRNA^{T1}, |
| | | | | mEGFP⁵¹⁰⁻⁷²⁰ |
| pSL510 | Donor | H3 | PheS-ampR | oriT, kanR, P_{J23119}-gRNA^{T1}, |
| | | | | mEGFP⁵⁰⁰⁻⁷²⁰ |
| pSL511 | Donor | H3 | PheS-ampR | oriT, kanR, P_{J23119}-gRNA^{T1}, |
| | | | | mEGFP⁴⁹⁰⁻⁷²⁰ |
| pSL512 | Donor | H3 | PheS-ampR | oriT, kanR, P_{J23119}-gRNA^{T1}, |
| | | | | mEGFP⁴⁸⁰⁻⁷²⁰ |
| pSL681 | Donor | H3 | PheS-ampR | oriT, kanR, P_{J23119}-gRNA^{T1}, |
| | | | | mEGFP⁴⁷⁰⁻⁷²⁰ |
| pSL1060 | Recipient | H1, H3 | NsrR-PheS | GmR |
| pSL1065 | Donor | H1, H3 | HygR-SacB | oriT, kanR, P_{J23119}-gRNA^{T1}, |
| | | | | mEGFP¹⁻²⁵¹ |
| pSL1062 | Donor | H3 | NsrR-PheS | oriT, kanR, P_{J23119}-gRNA^{T2}, |
| | | | | mEGFP¹⁹⁸⁻⁵¹⁷ |
| pSL1066 | Donor | H3 | HygR-SacB | oriT, kanR, P_{J23119}-gRNA^{T1}, |
| | | | | mEGFP⁴⁵⁴⁻⁷²⁰ |
| pSL1064 | Donor | H1, H3 | HygR-SacB | oriT, kanR, P_{J23119}-gRNA^{T1} |
| pSL1063 | Donor | H3 | HygR-SacB | oriT, kanR, P_{J23119}-gRNA^{T1} |
| pSL1107 | Donor | H3 | NsrR-PheS | oriT, kanR, P_{J23119}-gRNA^{T2} |
| pSL1086 | Recipient | H1, H3 | NsrR-PheS | pLacIQ-p15A, GmR |

Construction of the in vivo DNA stitching system: Construction of the helper plasmid and the host recipient strains. In the related MAGIC cloning system (Li, M. et al. Nat. Genet. 37, 311-319 (2005)), the recipient cells contain a helper plasmid pML300, which harbors an inducible λ-red and a temperature-sensitive origin of replication (pSC101-ori^{TS}) MAGIC recipient cells also have a genomically-integrated inducible I-SceI endonuclease allele. To implement recursive cutting and homologous recombination, a different helper plasmid containing λ-red and Cas9 is needed. To construct such a helper plasmid, pML300 was first digested and ligated into a multi-cloning site (MCS) via HindIII and NheI, which resulted in pSL270. One DNA fragment containing araC-P_{araBaAD}-Cas9 was constructed using Gibson Assembly, and then cloned into pSL270 using PacI and XhoI restriction sites to create the helper plasmid pSL359. This plasmid contains a Rhamnose-inducible λ-red recombination system (P_{rhaBAD}-red), an arabinose inducible endonuclease (P_{araBAD}-Cas9), a pSC101-ori^{ts}, and a spectinomycin resistance marker (SpR). pSL359 was then transformed into BW28705 to create the recipient host cells BW28705/pSL359. As alternative recipient strain, RE1133 (Egbert et al. Nucleic Acids Research, vol. 47 (6) 8 Apr. 2019, Pages 3244-3256) [cmR::mutS pTet2-gam-bet-exo-dam/tetR::bioA/B ilvG+dnaG.Q576A lacIQ1 Pcp8-araE ΔaraBAD pConst-araC ΔrecJ ΔxonA Pₖₘ-cymR-Cas9::bioC] was used without a helper plasmid. RE1133 contains an tetracycline-inducible λ-red recombination system (pTet2-gam-bet-exo-dam/tetR::bioA/B) and an cumate-inducible Cas9 endonuclease (Pkm-cymR-Cas9::bioC).

Construction of swapping cassettes: A swapping cassette is defined as the stretch of DNA on the donor and recipient plasmids that participates in a DNA swap: the cassette on the recipient plasmid is replaced by the cassette originally found on the donor plasmid via homologous recombination. In order to recursively select for cassette swapping in vivo, each cassette is engineered to contain both a selectable and counter-selectable marker. A selectable marker in the donor cassette and a counter-selectable marker in the recipient cassette is needed in every round. To implement such dual selection strategy, two different selection cassettes were constructed from the following sources by standard cloning methods: 1) PheS Gly²⁹⁴ (D,L-p-Cl-Phe sensitivity) (Kast, P. Gene 138, 109-114 (1994)) SacB (sucrose sensitivity) (Pelicic, V. et al. J. Bacteriol. 178, 1197-1199 (1996)), 3) HygR (hygromycin resistance) (Gritz, L et al. Gene 25, 179-188 (1983)) with an EM7 bacterial promoter, 4) NsrR (nourseothricine resistance) Gene 62, 209-217 (1988). One cassette was constructed to contain PheS Gly²⁹⁴ and NsrR, and the second cassette was constructed to contain HygR and SacB. Several other selection cassettes were also constructed to perform a number of experiments to characterize the in vivo stitching system: 5) ZeoR (zeocin resistance) (Drocourt, D. Nucleic Acids Res. 18, 4009-4009 (1990)), 6) ampR (ampicillin resistance), and 7) CmR (chloramphenicol resistance). In some cases, one cassette was constructed to contain PheS Gly²⁹⁴ and NsrR, and the second cassette was constructed to contain HygR and SacB.

Construction of the backbones for donor and recipient vectors: The donor vector was constructed to contain the following important components: kanR, oriT, R6K ori7, and a constitutive gRNA expression cassette driven by a strong bacterial promoter J23119 (Standage-Beier, K. Et al ACS Synth. Biol. 4, 1217-1225 (2015)). The swapping region was reconfigured to generate an T1(F)-H1-T2(R)-T2(F)-H3-T1(R) fragment, where T1 (5′-GGGGCCACTAGGGACAGGATtgg-3′ (SEQ ID NO: 37) and T2 (5′-CAGGCGGGCTCACCTCCGTGtgg-3′ (SEQ ID NO: 38)) are two unique target sequences for CRISPR-Cas9 cutting, and H1 (5′-CGAGGGCTAGAATTACCTACCGGCCTCCACCATGCCTGCG-3′ (SEQ ID NO: 39), and H3 (5′-GTACGGGCAACCCGAGAAGGCTGAGCCTGGACTCAACGGGTTGCTGGGTGGACT CCAGACTCGGGGCGACGACTCTTCACGCGCAGAGCAAGGGCGTCGAGCGGTCGT GAAAGTCTTAGTACCGCACGTGCCGACTCACTGGGGATATTGCCTGGAGCTGTAC CGTTCTAGGGGGGGGAGGTTGGAGACCTCCTCTTCTCACGACTGGACCCGCGAG GGCCGCGTTGCCGGTTCCCCCAGAGGCTGAAGAACAAGGGCTTACTGTGGGCAG GGGGACGCCCATTCAGCGGCTGGCGCTTT-3′(SEQ ID NO: 40)) are homology sites for homologous recombination, and (F) and (R) indicate whether the DNA fragment is included in the forward or reverse (reverse complement) orientation. A selection cassette (HygR-SacB or NsrR-PheS) is inserted between T2(R) and T2(F) to generate stitching-ready donor plasmids. In some cases, the swapping region of the donor plasmids was reconfigured to generate an T2(F)-T1(R)-T1(F)-H3-T2(R) fragment with the selection cassette (HygR-SacB or NsrR-PheS) inserted between T1(R) and T1(F). Both of gRNA target sites are positioned in the appropriate orientation to ensure the distance between the loci of double strand break and homology regions as short as possible. H3 is a 300-bp synthetic DNA fragment that is used as one homology arm in all rounds of assembly. H1 is a homology region that is used in the first round of in vivo stitching and can be incorporated in the donor backbone or introduced as part of the first oligonucleotide stitched. Other homology regions (H2, H4, H5, etc.) are introduced into donor plasmids as part of subsequent oligonucleotides, and overlap with the homology region of the previous oligonucleotide in an assembly to enable seamless stitching. The entry recipient vector contains a selectable marker (GmR) and replication origin (ColE1). The swapping region is modified to a H1-T1(R)-T1(F)-H3 configuration and a selection cassette (HygR-SacB or NsrR-PheS) was cloned between T1(R) and T1(F).

Test of endonuclease cutting and homologous recombination efficiency: To test whether the CRISPR/Cas9 system provides precise DNA cleavage and promotes homologous recombination, stitching operation was completed in the presence or absence of a targeting gRNA, Cas9, and kred. A recipient plasmid, pSL402 was transformed into three different recipient host cells to construct: 1) BW28705/pSL402 (−)red/−Cas9), 2) BW28705/pML300/pSL402 (+)red/−Cas9), 3) BW28705/pSL359/pSL402 (+λred/+Cas9). Two different donor plasmids, pSL414 and pSL415, were then transformed into BUN20 to create BUN20/pSL414 and BUN20/pSL415, which contains a functional and a mock gRNA unit, respectively. Each of the two donor strains was mated with each one of the three different types of recipient cells as described above. Cells were then diluted and plated on the selection plates (C1-Phe+Gm+Cm+0.2% Glucose) to recover recombinant clones in 37° C. overnight. Colonies were counted to quantify the recombination events.

Assembly of the mEGFP gene in liquid: Three fragments of a mEGFP gene were generated by PCR. The first fragment, containing a constitutive promoter pJ23100, a ribosome binding site, and nucleotides 1-251, was cloned into a donor backbone to create pSL485. The second fragment containing nucleotides 198-517 was cloned into a donor vector to create pSL486. The third fragment containing nucleotides 454-720 and rrnB T1 terminator was cloned to a donor vector to create pSL488. All three of donor vectors were transformed into BUN20 and grown on LB+Kan plates overnight at 37° C. An entry recipient vector pSL398 was transformed into BW28705/pSL359 and grown on a LB agar plate containing gentamicin, spectinomycin and glucose. A clone of the donor BUN20/pSL485 (D1) and the recipient BW28705/pSL359/pSL398 (R0) were grown in appropriate liquid media in 37° C. and 30° C. overnight, respectively. Cells (1 ml) from both donor and recipient were then spun down, mixed, and resuspended in 1 ml pre-warmed LB+Ara+Rha liquid media. Following −4 hours incubation at 30° C. without shaking, serial dilution was performed in the mating cultures and cells were plated on 6% Suc+Carb+Gm +Sp+0.2% Glucose to select recombinants (R1). Colony PCR was conducted to confirm a correct R1 clone, which was then inoculated in LB+Gm+Sp+0.2% Glucose in 30° C. overnight. Freshly cultured donor cells that contain pSL486 (D2), were then spinned down, mixed, and resuspended with R1 in liquid mating media in 30° C. for ˜4 hours. Recombinant clones were selected by plating on C1-Phe+Hyg+Gm+Sp+0.2% Glucose. A correct clone (R2) that was confirmed by colony PCR, was grown in LB+Gm+Sp+0.2% Glucose in 30° C. overnight. As in previous rounds, D3 (BUN20/pSL488) and R2 cells were mixed and resuspended in the liquid mating media. A serial dilution was conducted and cells were plated on 6% Suc+Carb+Gm+Sp+0.2% Glucose. Plates from each round of assembly were imaged under UV light to count the proportion of GFP fluorescent colonies. After each round of assembly, the selected clones were picked and plasmids were purified for diagnostic restriction digestion and Sanger sequencing.

Test of the effect of homology length on stitching: To test the impact of homology length on the stitching accuracy, a series of plasmids were constructed to contain different sizes of homology to the 2nd mEGFP fragment in pSL486: 1) pSL684 (0 bp), 2) pSL685 (10 bp), 3) pSL510 (20 bp), 4) pSL511 (30 bp), 5) pSL512 (40 bp), 6) pSL681 (53 bp). These plasmids along with pSL488 (63 bp) were transformed into donor host cells to construct a group of D3 to mate with recipient cells containing R2. Cells from each mating pair were diluted and plated on the selection plates (6% Suc+Carb+Gm+Sp+0.2% Glucose). Colonies were recovered overnight and examined under UV light to observe fluorescence. The number of fluorescent and non-fluorescent colonies were counted to calculate the percentage of correct assembly.

Arrayed assembly of mEGFP: All strains were arrayed on agar plates in a 384-format. First, BUN20/pSL1065 (D1) and BW28705/pSL359/pSL1060 (R0) were arrayed and mixed together using SINGER ROTOR HDA on a pre-warmed mating plate (LB+Ara+Rha), and grown in 30° C. for −5 hours. Mated cells were then transferred onto a first selection plate (C1-Phe+Hyg+Gm+Sp+0.2% Glucose). Recombinant clones (R1) were enriched in 30° C. overnight before being transferred on a pre-mating plate (LB+Hyg+Gm+Sp+0.2% Glucose), which optimizes the growth of the assembled plasmids for the next round. Fresh overnight arrays of BUN20/pSL1062 (D2) were then mated with R1 on a mating plate. Mated cells were then transferred onto the first selection (LB+Nat+Gm+Sp+0.2% Glucose) to select recombinant clones in 30° C. overnight. Selected clones (R2) were then transferred on a pre-mating plate (6% Suc+Nat+Gm+Sp+0.2% Glucose). Fresh overnight arrays of BUN20/pSL1066 (D3) were then mated with R2 on the mating plate. The final assembly products (R3) were selected following the selection on C1-Phe+Hyg+Gm+Sp+0.2% Glucose and then LB+Hyg+Gm+Sp+0.2% Glucose. Plates from each round of assembly were imaged on a UV transilluminator under UV light to monitor GFP fluorescence. Over the course of assembly, selected clones were picked and plasmids were purified for diagnostic restriction digestion and Sanger sequencing.

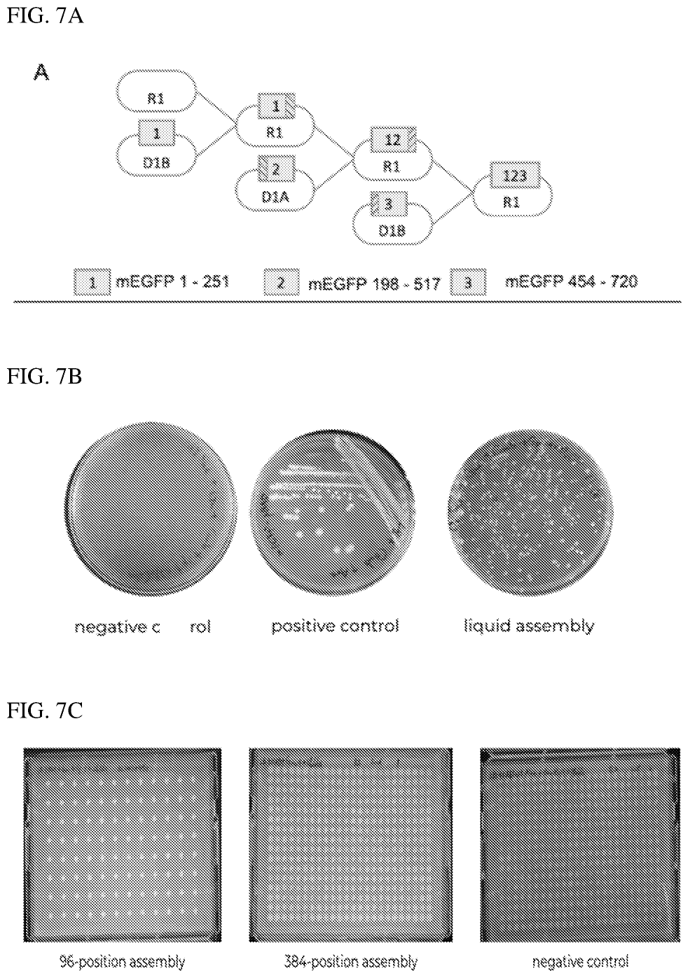

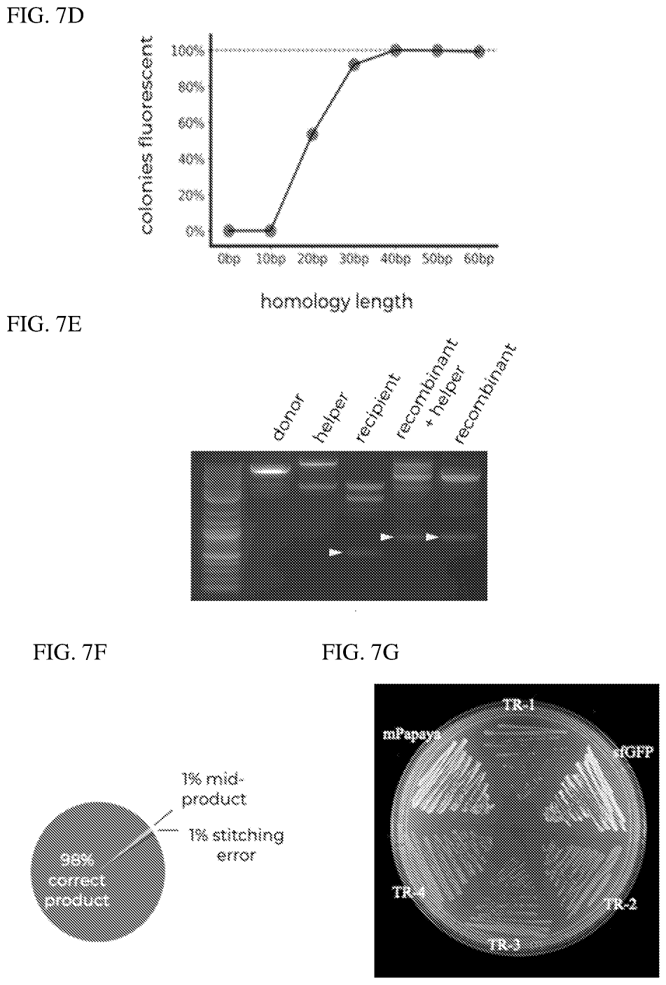

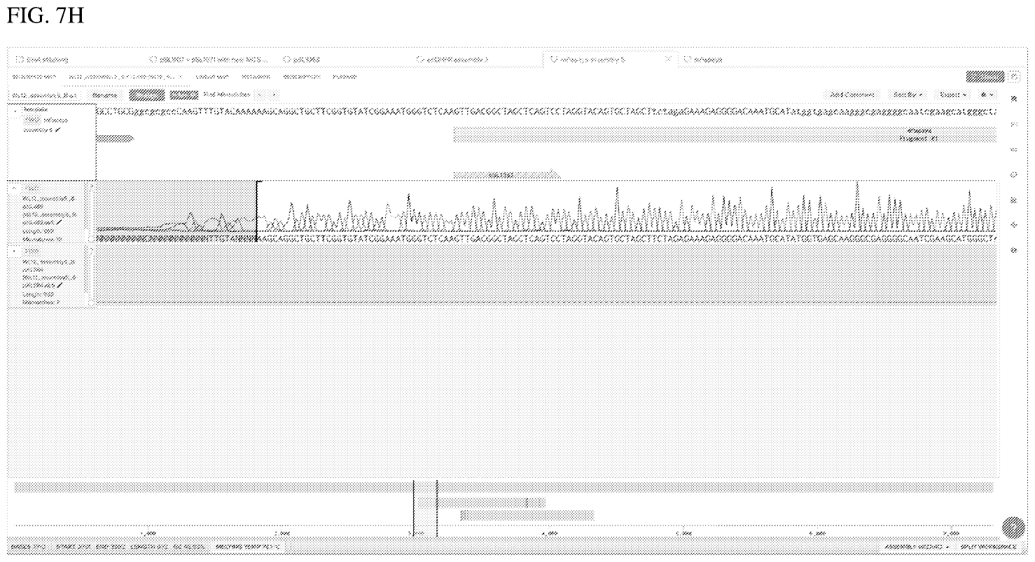

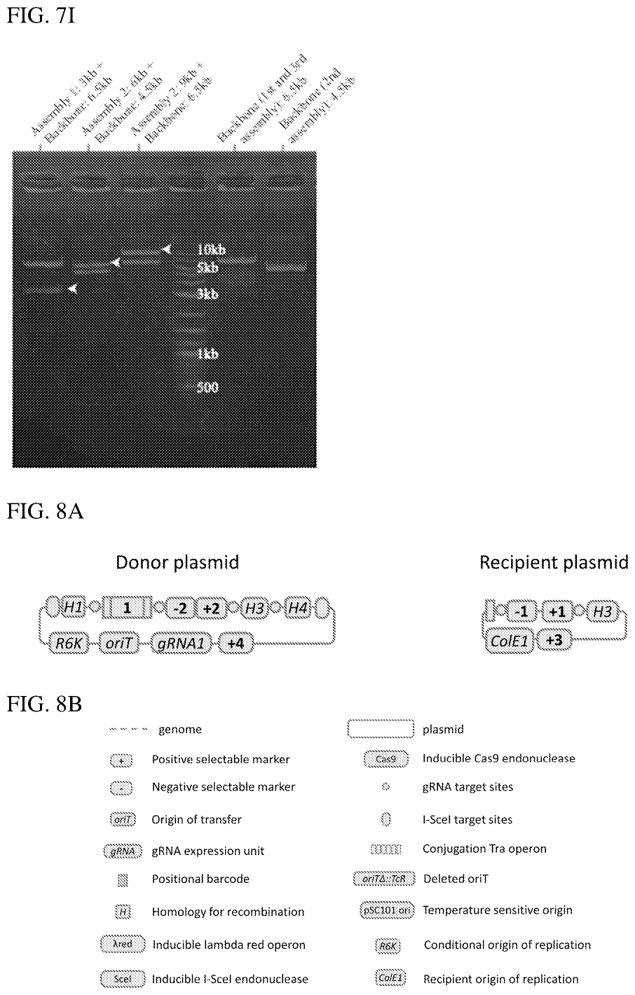

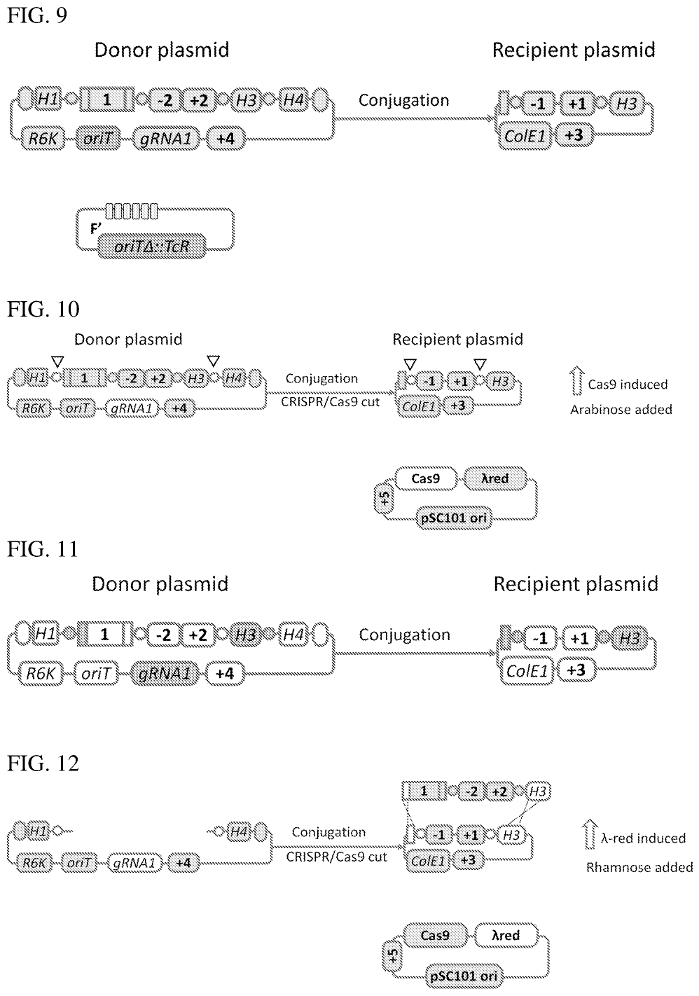

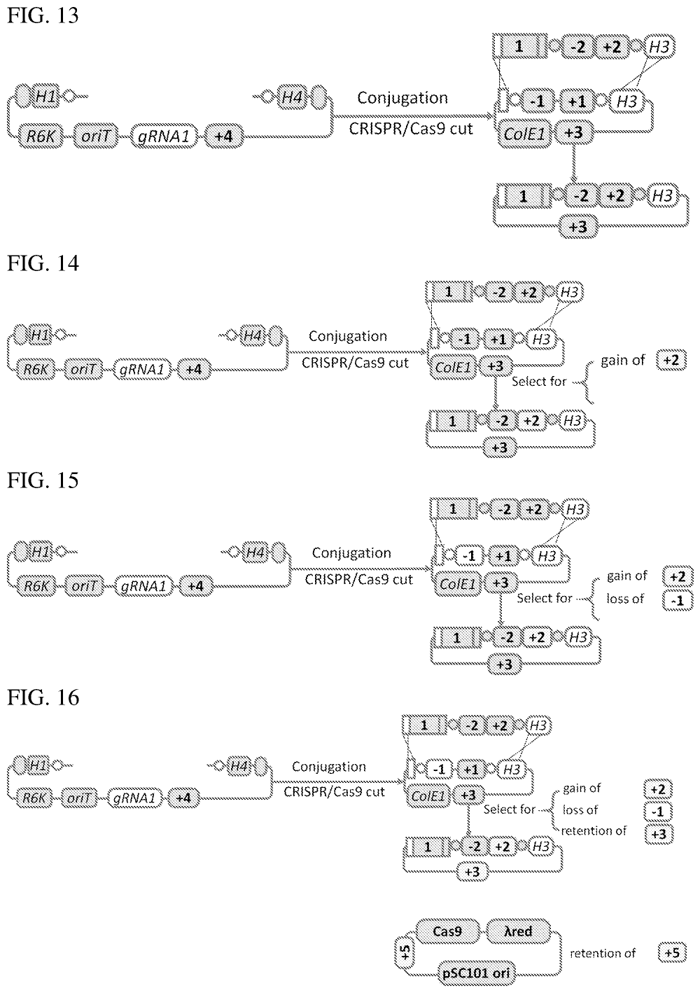

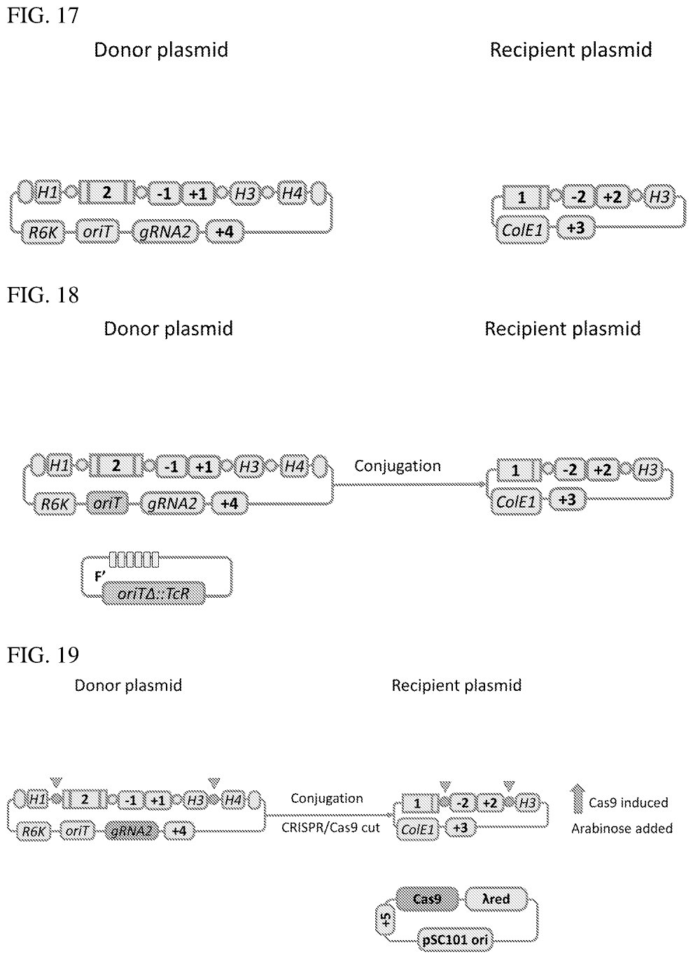

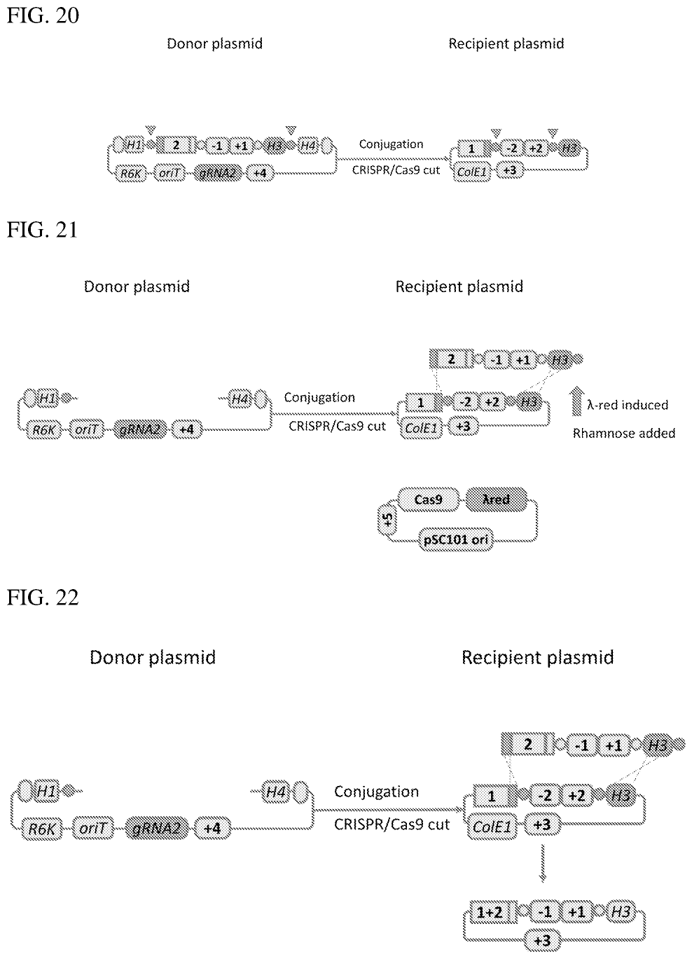

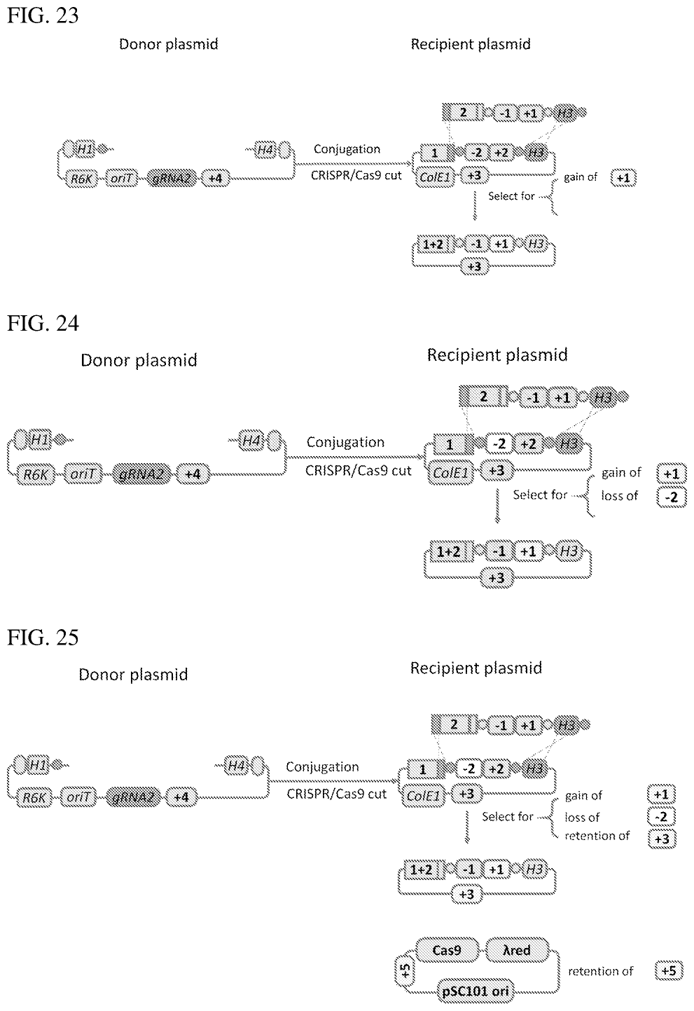

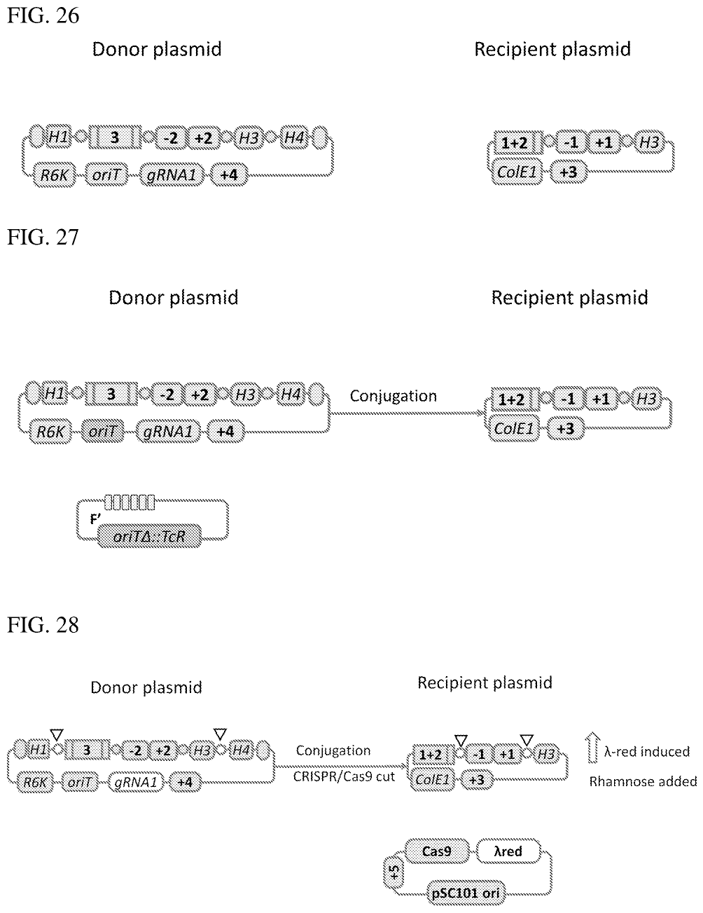

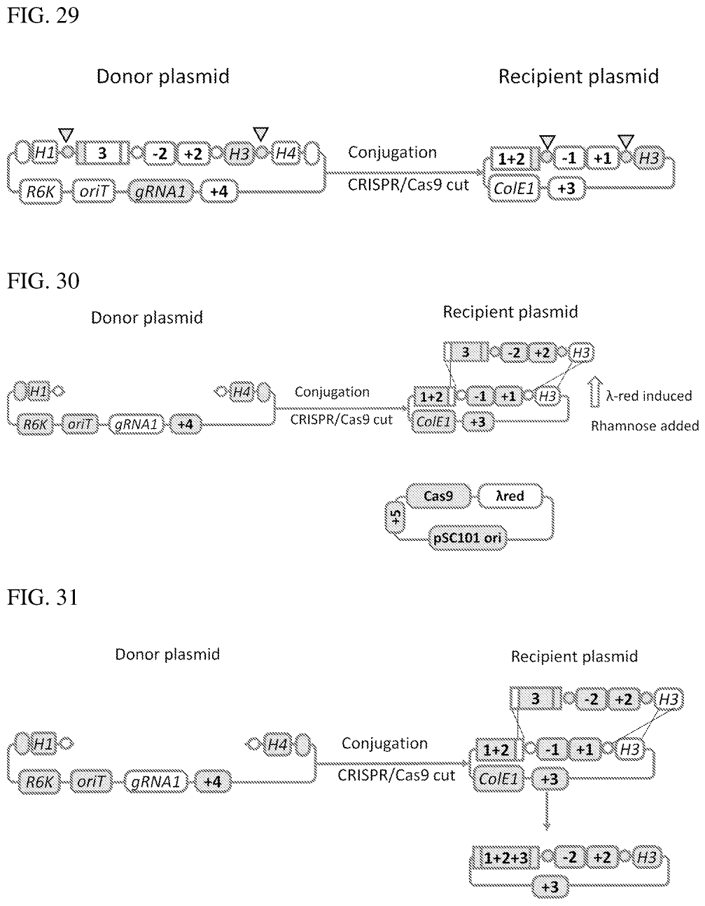

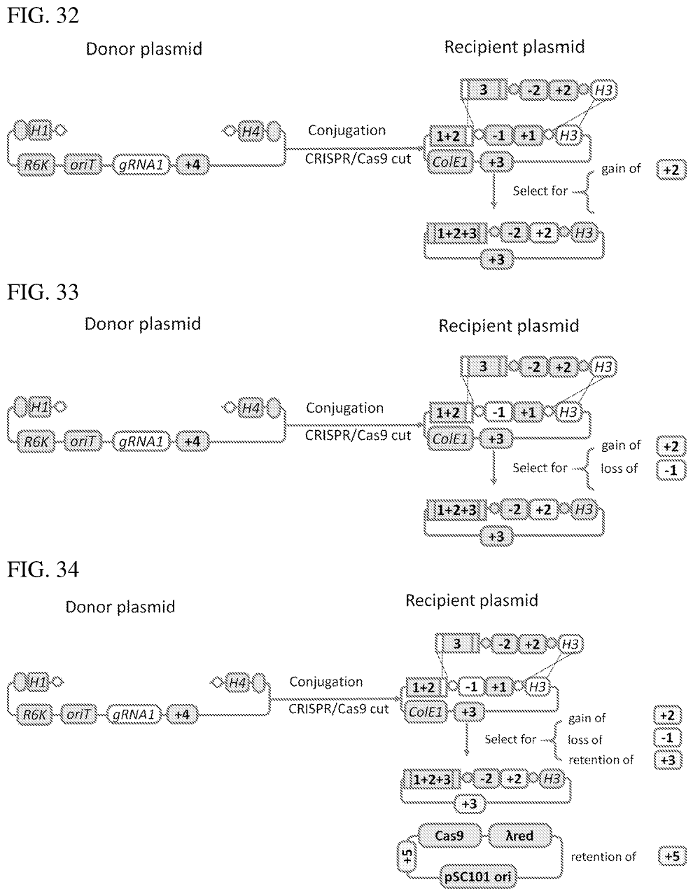

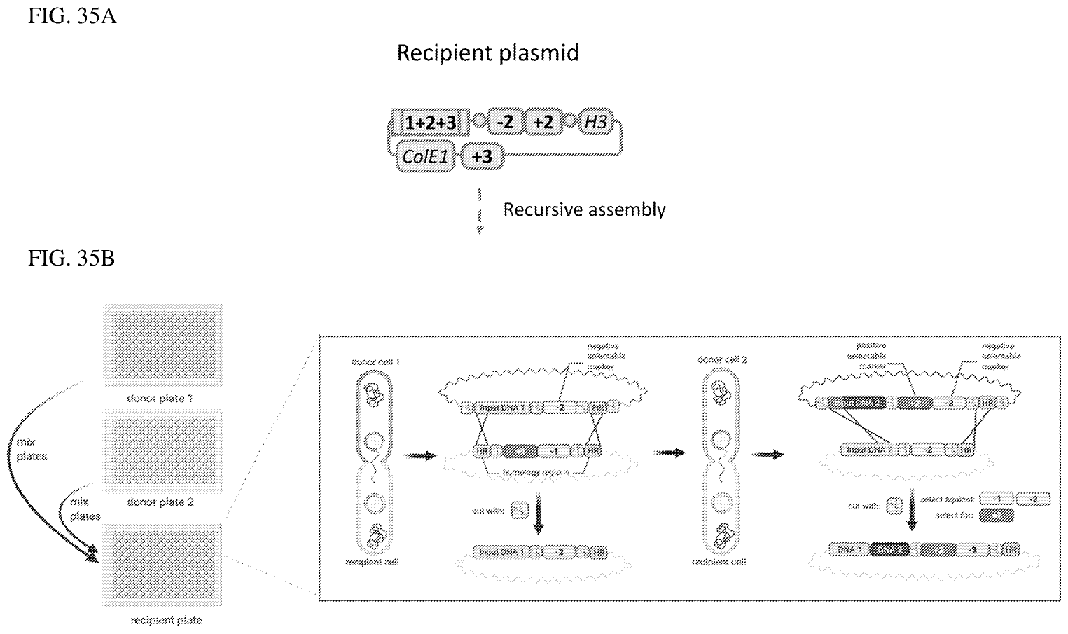

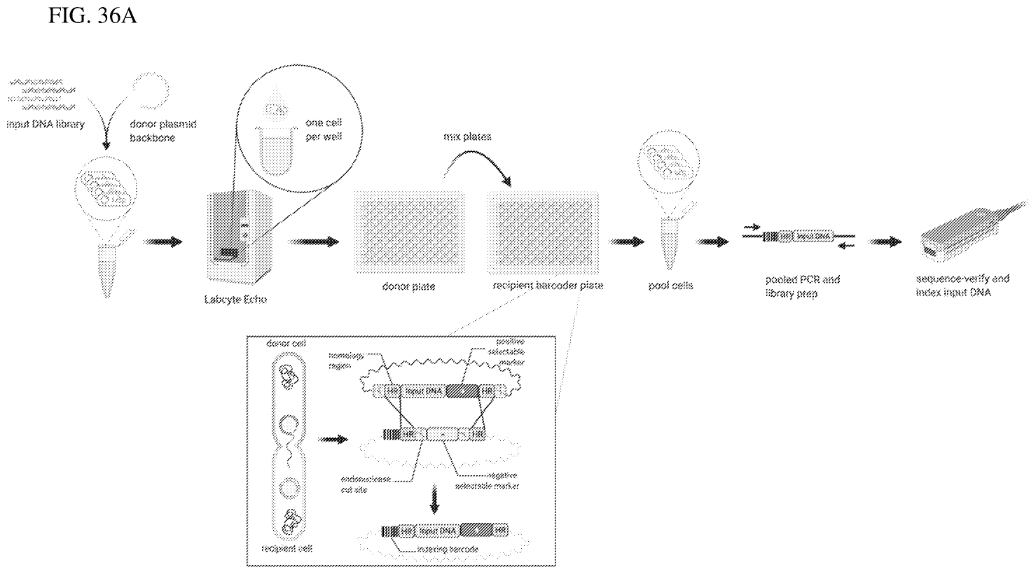

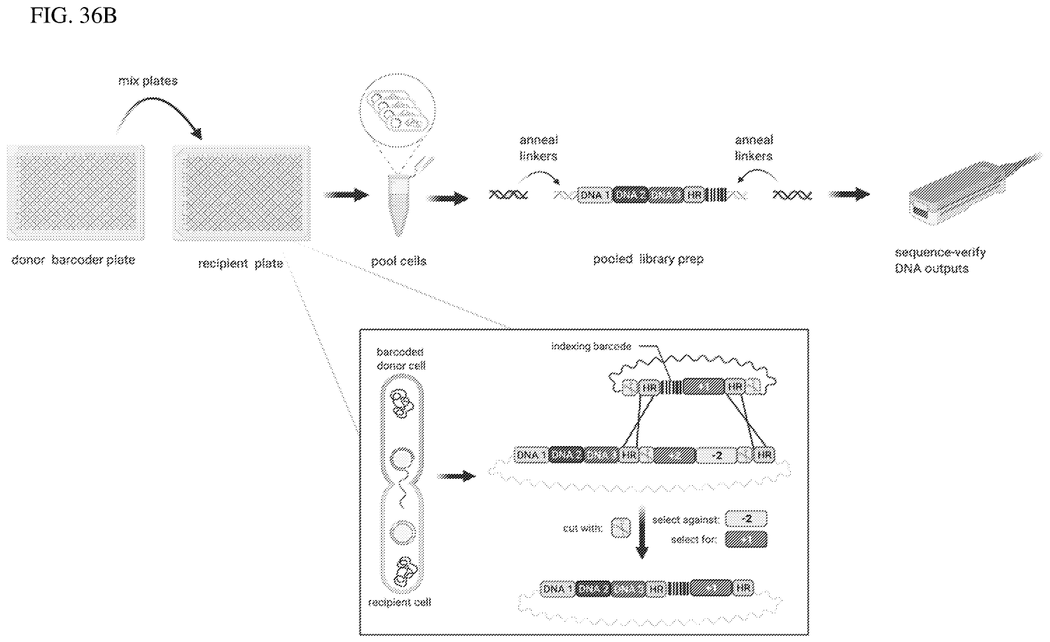

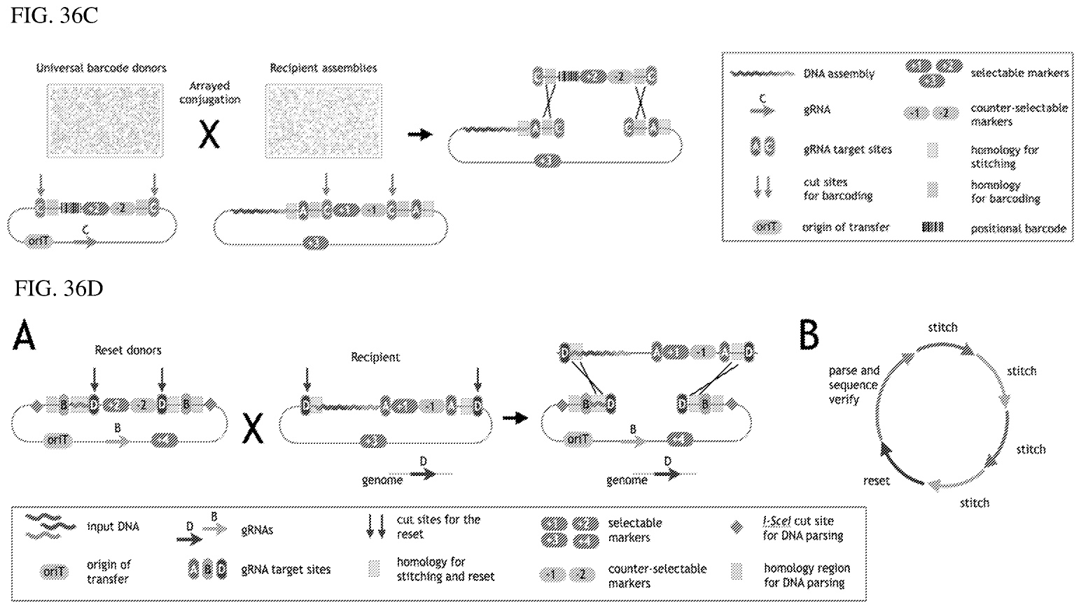

Arrayed assembly of 12 genes using pooled oligonucleotides: Nine different serine/tyrosine recombinases and 3 different fluorophores (mPapaya, mPlum, sfGFP) were chosen for assembly. To generate a list of oligonucleotides necessary for each gene assembly, a python script was written that inputs a FASTA file containing the genes to be synthesized and outputs a list of oligonucleotides to be ordered from a commercial supplier (IDT oPool), with user defined variables that include the synthesized oligonucleotide length, the minimum homologous overlap length between adjacent oligos, and the maximum homologous overlap length. DNA hairpins and/or repeats may interfere with the homologous recombination machinery and reduce assembly fidelity, although no quantitative studies to this effect are known. Therefore, for each gene, these regions were identified using the Primer3 python extension (Untergasser, A. et al. Nucleic Acids Res. 35, W71-W74 (2007)) and a nucleotide distribution uniformity metric. Each nucleotide position is scored, and user-defined thresholds are used to extend the homology region if smaller homology regions are likely to contain interfering elements. Once the oligonucleotides necessary for each gene assembly have been determined, on each end restriction sites (NotI and AscI) are added that are used to clone oligonucleotides into donor vectors and round-specific priming sites. Priming sites allow oligonucleotides from a specific round of a parallel gene assembly to be amplified together and parsed by the in vivo parsing platform. Round-specific primers are chosen from a primer list that has been previously designed to reduce the possibility of cross reactivity between primers (lower the number of undesired PCR products) when used on large oligonucleotide pools. (Kosuri, S. et al. Nat. Biotechnol. 28, 1295-1299 (2010)). Using this python script, each gene was split into five ˜300 bp oligonucleotides with 50-70 bp of homology between subsequent oligonucleotides. PCR amplified oligonucleotides were inserted into the donor plasmids using restriction digest and ligation. For the first round of assembly of each gene, the oligonucleotides were amplified and cloned into the donor backbone pSL1064, which includes a 40 bp starting H1 region that is homologous to the one in the entry recipient plasmid pSL1060. Oligonucleotides to be added in additional odd rounds of stitching (e.g. 3, 5, 7, 9) were cloned into pSL1063, which contains the same elements as pSL1064, except it lacks a H1 homology region. Oligonucleotides to be added in even rounds of stitching (e.g. 2, 4, 6, 8) were cloned into pSL1071. PCR amplified oligonucleotides and the donor plasmids were digested with AscI and NotI for 4 hours at 37° C. The digested products were then size-selected and purified via gel extraction using Zymoclean Gel DNA Recovery kits. 0.02 pmol of digested donor plasmid and 0.06 pmol of digested oligonucleotides were ligated by mixing with 1 ul of T4 ligase and incubating at 22° C. for 1 hour. The ligated donor plasmids were transferred into BUN20 donor strains using standard bacterial transformation protocols. 2 ul of the ligation product was added to 50 ul of chemically-competent BUN20 donor strains. The mixture was then incubated on ice for 30 minutes, heat-shocked at 42C for 30 seconds, and incubated on ice again for 3 minutes. Cells were resuspended in 950 ml of NEB SOC recovery medium and recovered at 37° C. for 1 hour. Cells were then plated onto selection plates (LB+Hyg for odd round donor plasmids, LB+Nat for even round donor plasmids) and incubated overnight at 37° C. Resulting colonies containing cloned oligonucleotides were randomly selected and arrayed in 96-well plates. Arrayed oligonucleotide libraries were parsed and sequence verified using the in vivo DNA parsing system. Each plate was mated to two different recipient barcode plates. For the oligonucleotide plates in the odd assembly rounds, the donor backbones (pSL1063 or pSL1064) contain the HygR-SacB cassette. Following mating with BPS recipient arrays on LB+Ara+IPTG agar for −3 hours in 37° C., the recombinant plasmids were selected on LB+Hyg+Gm+Rha+Ara plates in 37° C. overnight. For even-round oligonucleotide arrays, which contain the NsrR-PheS cassette, the recombinant plasmids were selected on LB+Nat +Gm+Rha+Ara plates in 37° C. overnight following mating with BPS collections. To assemble the 12 genes, BUN20 donor strains carrying the first round oligonucleotides were first mated with the RE1133 recipient strain carrying the pSL1086 recipient plasmid. 50 ul of each overnight culture was mixed, spun down at 8000 rpm for one minute, resuspended in 50 ul of LB, and incubated at 37° C. for 30 minutes. The mated cells were then plated onto LB+aTC+cumate plates and incubated for 4 hours at 37° C. to induce Cas9 and λ-red. To isolate cells that carry the recombinant recipient plasmid with the first round oligonucleotide, the mated cells were streaked out onto LB+Hyg+Gm+IPTG agar plates and incubated overnight at 37° C. Any un-recombined donor plasmids are quickly removed as the R6Kγ origin on the donor plasmid is non-functional in the pir⁺ RE1133 recipient strain background. Colonies from the selection plates were further purified by selecting onto LB+Hyg+Gm+IPTG+4CP to remove any remaining un-recombined pSL1086 recipient plasmid. To assemble the second rounds of oligonucleotides, purified colonies carrying the recombinant recipient plasmid with the first round oligonucleotide were then mated with BUN20 donor strains carrying the second round oligonucleotides. The same procedure was used as the first assembly except that cells carrying the recombinant recipient plasmids were selected on LB+Nat+Gm+IPTG and further purified on LB+Nat+Gm+IPTG+6% sucrose. For all subsequent assembly steps, the same assembly procedures were used with LB+Hyg+Gm+IPTG+6% sucrose being used for selection for odd-round assemblies and LB+Nat+Gm+IPTG being used for even-round assemblies. This process was repeated five times until all 12 genes were fully assembled (FIG. 7G). Sequences of the assembled products were verified to be the correct sequence using Sanger sequencing (FIG. 7H) and an Oxford Nanopore MinION sequencer.

Assembly of a 9 kb fragment: A 9 kb DNA block from chromosome II positions 41489 to 50489 of the BY4741 Saccachromyces cerevisiae strain was assembled. Using the same Python script used for the assembly of the 12 genes, the 9 kb block was split into three ˜3 kb DNA blocks with 50-75 bp of homology between subsequent DNA blocks. These 3 DNA blocks were PCR amplified using genomic DNA from the yeast strain BY4741 as DNA template. Genomic DNA was extracted using MasterPure Yeast DNA Purification kit. First, second, and third DNA blocks were inserted into donor plasmids pSL1064, pSL1063, and pSL1107, respectively using AscI/NotI restriction digest and T4 ligation. The resulting ligated products were then transformed into BUN20 donor strains using standard bacterial transformation procedures. After the donor plasmids were sequence verified using Sanger sequencing, the DNA blocks were assembled in the RE1133/pSL1086 recipient strain. A donor strain carrying the first DNA block was mated with RE1133/pSL1086 and grown on LB+aTC+cumate at 37C for 4 hours. Cells carrying the recombinant recipient plasmid were selected on LB+Hyg+Gm+IPTG and further purified on LB+Hyg+Gm+IPTG+6% sucrose. Resulting colonies were then mated with donor strains carrying the second DNA block, selected for recombinant recipient plasmid on LB+Nat+Gm+IPTG, and purified on LB+Nat+Gm+IPTG+4CP. Finally, the resulting colonies were mated with donor strains carrying the third DNA block, selected for recombinant recipient plasmid on LB+Hyg+Gm+IPTG, and purified on LB+Hyg+Gm+IPTG+6% sucrose. Sequences of the assembled products were then verified to be the expected sequence using Oxford Nanopore MinION sequencer and gel electrophoresis (FIG. 7I).

Amplicon sequencing to parse an oligonucleotide library: To extract the recombinant plasmids, cells were scraped from the selection plates and mini prepped using Plasmid Plus Mini Kit (QIAGEN). The plasmid DNA was then quantified and diluted to ˜1 ng/μl, which is approximately 1.5e6 copies per unique barcode-barcode pair on a 96 arrayed mating plate. A two-step PCR was performed, as described (Levy, S. F. et al. Nature 519, 181-186 (2015)) with modifications. First, 4-5 cycles PCR with OneTaq polymerase (New England Biolabs) was performed using the forward (pBPS_fwr) and reverse (pBPS_rev) primers listed in Table 1. ˜1 ng of recombinant plasmid DNA was amplified in a single 50 μl PCR reaction. Primers for the first step PCR have this general configuration:

pBPS_fwr:
(SEQ ID NO: 41)
ACACTCTTTCCCTACACGACGCTCTTCCGATCTNNNNNNNNXXXXXXttc
ggttagagcggatgtg
pBPS_rev:
(SEQ ID NO: 42)
GTGACTGGAGTTCAGACGTGTGCTCTTCCGATCNNNNNNNNXXXXXXXXX
aggtaacccatatgcatggc.

The Ns in these sequences correspond to any random nucleotide and are used in the downstream analysis to remove skew in the counts caused by PCR jack-potting. The Xs correspond to a one of several multiplexing tags (for example, the multiplexing tags in Table 1 above), which allows different samples to be distinguished when loaded on the same sequencing flow cell. Examples of multiplexing tags are the underlined sequences in Table 1. The lowercase sequences correspond to the priming sites on the recombinant plasmids. The uppercase sequences correspond to the Illumina Read 1 or Read 2 sequencing primer. The PCR products were purified using NucleoSpin columns (Macherey-Nagel) and eluted into 33 μl water. A second 23-25 cycles PCR was performed with PrimeStar HS polymerase (Takara), with 33 μl of cleaned product from the first PCR as template and 50 μl total volume per tube. Primers for this reaction were the standard Illumina TruSeq dual-indexed primers (D501-D508 and D701-D712) listed in Table 2. PCR products were then cleaned using NucleoSpin columns. Amplicons from each mating plate were uniquely labeled with the customized multiplexing tags as well as Illumina standard indices. This quadruple-indexed strategy will not only increase the multiplexing capacity of the sequencing library but benefit the downstream analysis for amplicon chimeras. Cleaned amplicons were pooled and paired end sequenced on an Illumina MiSeq (2×300 bp) with 25% PhiX DNA spike-in. Sequencing reads were clustered into barcodes by using the Bartender. (Zhao, L., Bioinformatics 34, 739-747 (2017)).

Design of a recursive in vivo stitching technology: The in vivo stitching system takes advantage of the bacterial conjugation machinery and lambda-Red homologous recombination. The donor vector contains a conditional origin of replication from R6K ori7, which depends on a functional trans-acting factor π encoded by the gene pir1 or its relaxed copy-number control version, pir1-116 (Metcalf, W. Gene 138, 1-7 (1994)). This special origin allows the plasmid to be maintained in donor host cells that harbor a genomically integrated pir116 allele (e.g. BUN20), but not in recipient cells that lack this allele. Other important features in donor vectors include an oriT, a backbone marker (kanR), a constitutive gRNA expression cassette (gRNA^{T1} or gRNA^{T2}), and the swapping region. Within the swapping region, there are two pairs of unique gRNA target sites, a dual selectable cassette, and a common long homology sequence (300 bp) for each round of stitching.

The entry recipient vector contains an origin of replication (ColE1 or pLacIQ-p15A), a backbone marker (GmR), and the swapping region, which consists of homology sequences, two gRNA target sites and the dual selectable cassette. Some recipient host cells possess a helper plasmid containing a rhamnose-inducible λ-red recombination system and an arabinose-inducible Cas9 endonuclease. A temperature-sensitive mutant derivative of the replication origin (pSC101-ori^{TS}) provides a convenient means of curing the helper plasmid at 42° C. when the assembly is finished (Hashimoto, T. J. Bacteriol. 127, 1561-1563 (1976)). Other recipient host cells (RE1133) contain a genomically integrated tetracycline-inducible λ-red recombination system and cumate-inducible Cas9 endonuclease.

In each round, after the donor vector is transferred into recipient cells, both λ-red and Cas9 are induced in the presence of arabinose and rhamnose or cumate and tetracycline, depending on the recipient cell. The constitutively expressed gRNA^{T1} guides the Cas9 to both donor and recipient plasmids to generate double strand breaks. DNA breaks have been shown to greatly stimulate homologous recombination (see below) (Kuzminov, A. Microbiol. Mol. Biol. Rev. 63, 751 (1999)). The fragment from the donor contains a DNA of interest, two different gRNA target sites for the next round of assembly, a dual selectable marker, and the H3 homology region. The DNA for assembly is designed so that the first 50 bp is homologous to the last 50 bp of assembled sequences sitting on the recipient plasmid. This 50 bp serves as one homology arm for double crossover homologous recombination, with the other being H3. This swapping event results in a recombined recipient plasmid containing new DNA from the donor. Three different selections are implemented to guarantee the accuracy of the recombination: 1) selection against the counter selectable marker (PheS or SacB), 2) selection for the positive selectable marker (HygR or NsrR), and 3) selection for a marker on the recipient backbone (GmR). The selection for the helper plasmid as well as the repression of P_{araBAD} and P_{rhaBAD} promoters or the P_{km-cymR} and P_{Tet2} promoters are also enforced to prevent hyper-recombination and undesired DNA breaks. The alternating use of two different gRNAs and two different dual selectable cassettes enables recursive in vivo stitching to efficiently assemble new DNA fragments in a linear fashion, which results in the desired sequences with the only theoretical limit being the tolerable plasmid size. With liquid handling and multiplexed pinning robots, this platform is highly scalable, allowing thousands of parallel gene assemblies per round.

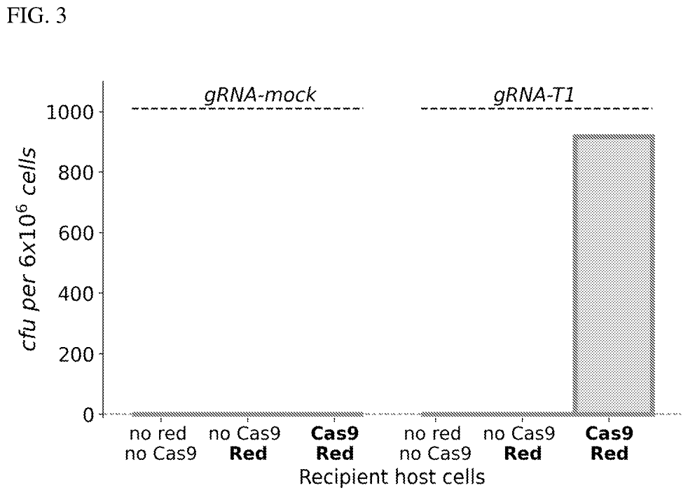

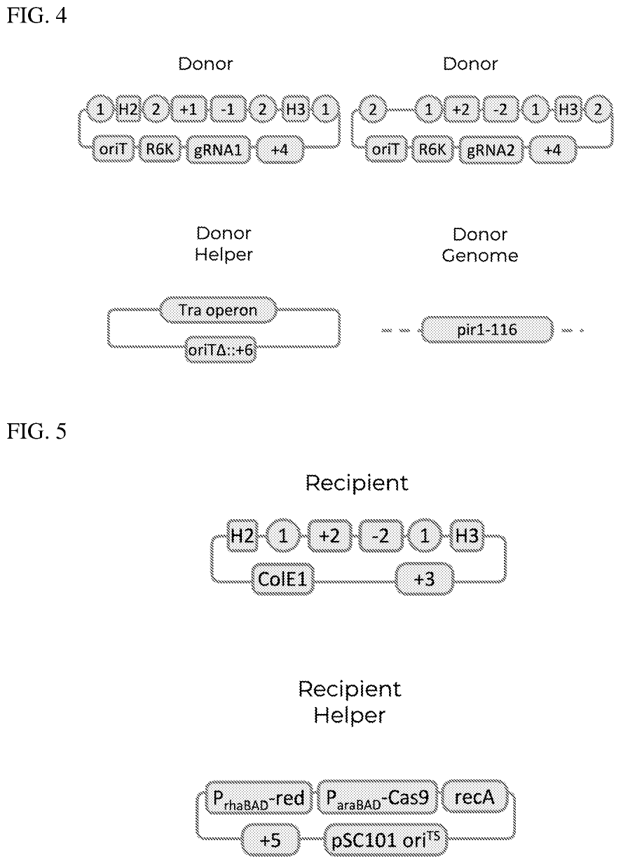

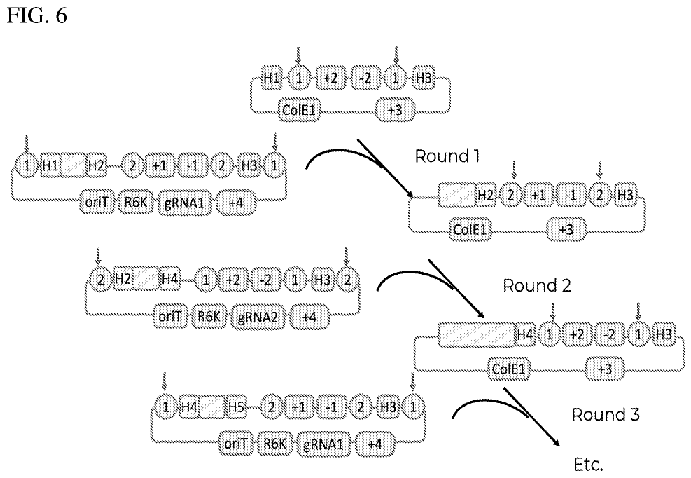

CRISPR-Cas9 can efficiently stimulate the in vivo stitching: To test whether the CRISPR/Cas9 system provides precise DNAcleavage and promotes homologous recombination, in vivo stitching was completed in the presence or absence of a targeting gRNA, Cas9, and λ-red. A recombinant plasmid was recovered when all three are present (FIG. 3), indicating that CRISPR/Cas9 facilitates DNA double strand breaks to enhance λ-red recombination efficiency.

Assembly of a functional fluorescent gene in liquid: To demonstrate the capability to assemble multiple fragments into a functional gene using the in vivo stitching system, three pieces of the mEGFP gene were constructed by PCR and cloned into appropriate donor backbones. Three fragments were then sequentially assembled into an entry recipient vector. After each round, the recovered clones were examined by both restriction digestion and Sanger sequencing to verify assembly accuracy before next round. To ensure that the helper plasmid was retained in each round, colony touch PCR and plasmid extraction were both performed. Green fluorescence was observed in all colonies (˜300) on the selection plates in the final round of assembly, indicating that assembly fidelity is high (FIGS. 7A-I).

The effect of homology length on stitching fidelity: To test how the fidelity of in vivo stitching depends on the length of homology between fragments, 7 donor vectors were constructed that contained the third fragment of the mEGFP assembly with different lengths of homology to the second fragment. After conjugation and recombination, cells derived from different conjugation/recombination events were plated on selection media and counted the fraction of fluorescent colonies. Results show that, in this example, 40 bp of homology is likely to produce error-free fusion products (FIGS. 7A-7I).

Multiplexed assembly of a functional fluorescent gene on agar: To demonstrate the capability to assemble a functional gene on agar plates, three pieces of the mEGFP gene were constructed by PCR and cloned into appropriate donor backbones. Three fragments were then sequentially assembled into an entry recipient vector in a 96- or 384-pin format. Green fluorescence was observed in 96/96 positions in the 96-pin format and 383/384 positions in the 384-pin format after the final round of assembly, indicating that the stitching fidelity on agar is comparable to that in liquid (FIGS. 7A-7I). Over the course of the assembly, the plasmids were recovered from various colonies (the entire colony was scraped) and examined by restriction digestion to verify assembly accuracy and/or retention of the helper plasmid. Typical digestion patterns of assembly end products indicated a clean recombinant plasmid without any observable undesired products (e.g. non-recombinant plasmid). To further characterize the stitching fidelity, 96 positions from a 384-position assembly were sequenced by Sanger sequencing. Sequencing products are derived from a colony PCR of a pipette tip touched to each colony. 94/96 colonies were found to contain the correct mEGFP sequence. One colony contained a mid-product (the first round assembly product) and one contained a stitching error (a large deletion).

Multiplexed assembly of genes from oligonucleotide pools. To demonstrate the capability to assemble a variety of DNA constructs from oligonucleotide pools, we constructed nine distinct serine/tyrosine recombinases and three distinct fluorphores (mPapaya, mPlum, sfGFP) from pools of 300 bp oligonucleotides purchased from IDT (oPools). Each gene was assembled from five oligonucleotides stitched together in series. Olignucleotide pools were integrated into the appropriated donor plasmid, transformed into donor bacteria, and bacterial pools were parsed into sequence-verified order arrays using methods described in Example 2: Methods for in vivo DNA analysis. Donor cell arrays were conjugated in series to recipient cells to assemble each gene with at least three-fold replication. Each assembly was determined to contain the correct DNA sequence by Sanger sequencing (FIG. 7G).

Assembly of long DNA. To demonstrate the capability to assemble with long DNA blocks and to produce long assemblies, we reconstructed a 9 kb segment of the Saccachromyces cerevisiae genome from three 3 kb blocks. The 3 kb blocks were amplified from genomic DNA, integrated into the appropriated donor plasmid, transformed into donor bacteria, and sequence verified. Donor cells were conjugated in series to recipient cells. To verify the correct assembly product at each assembly step, recombinant recipient plasmid was purified from recipient cells, linearized with a restriction enzyme, and analyzed by gel electrophoresis (FIG. 7H). Assembly products were also verified to be sequence correct by Sanger sequencing.

### Example 2: Methods for In Vivo DNA Analysis

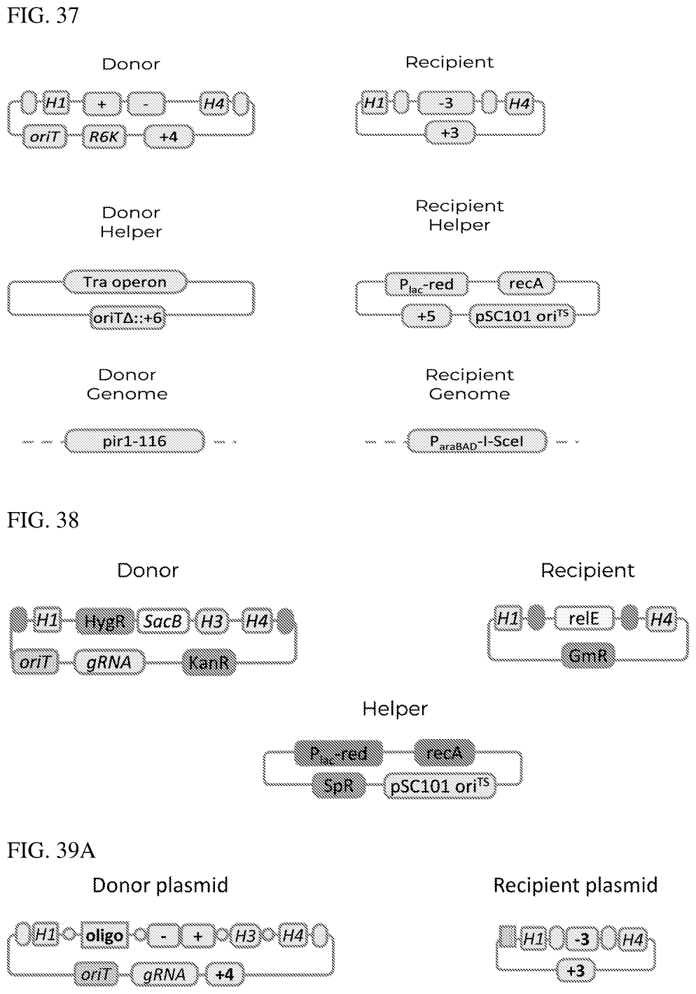

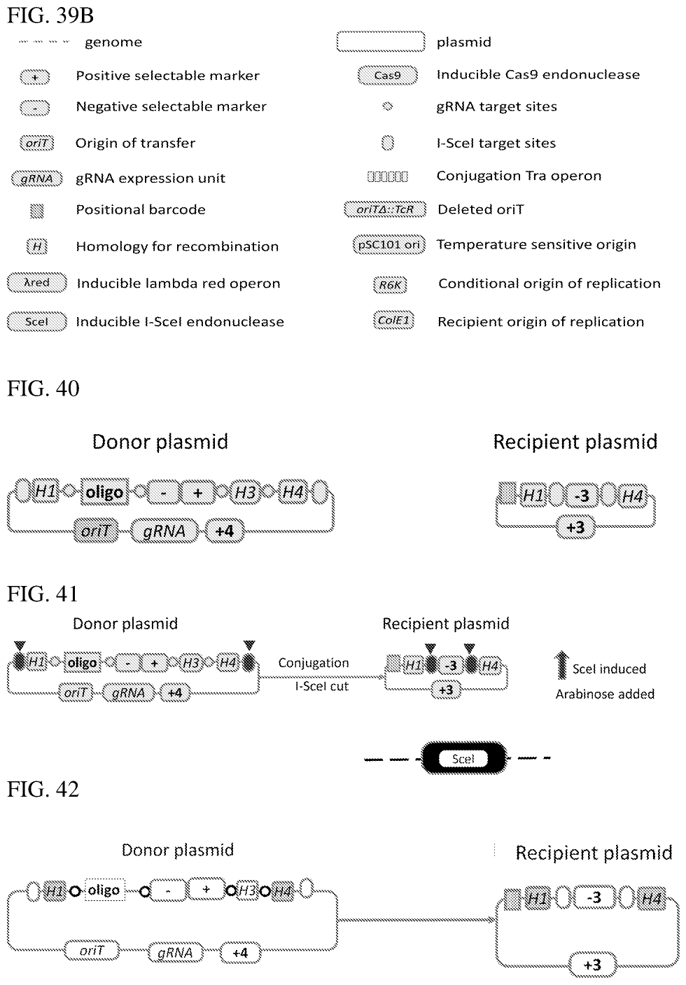

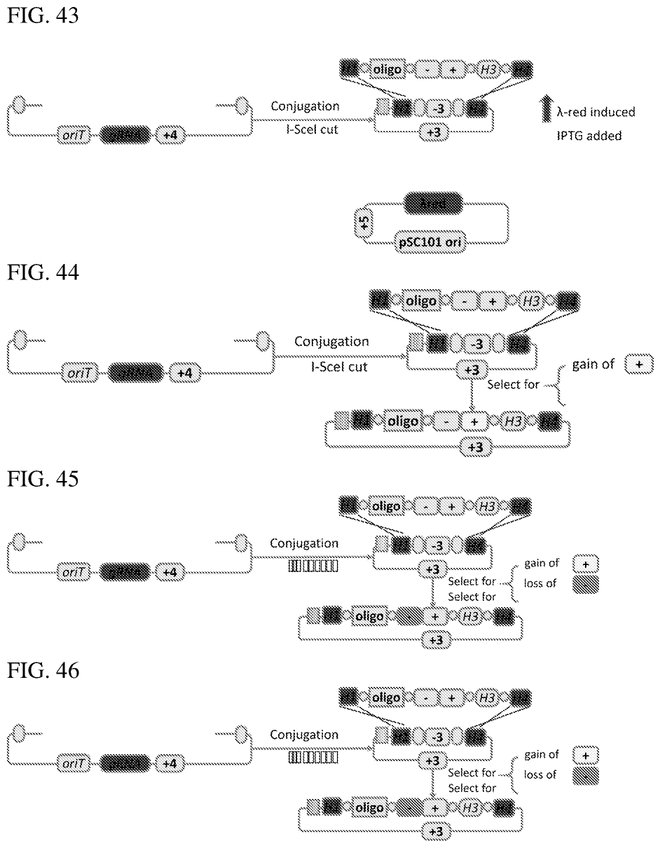

Plasmid sequences: The information about the plasmids used for in vivo DNA parsing can be found in Table 5 and FIG. 37.

TABLE 5
Plasmids used in in vivo parsing

| | Plasmid | Homology | | |
|---|---|---|---|---|
| Name | Type | Regions | Swapping cassette | Other features |
| pML104 | Helper | NA | NA | P_{lac}-red, recA |
| pSL937 | Recipient | H1, H4 | P_{rhaBAD}-relE | GmR |
| pSL438 | Donor | H1, H4 | HygR-SacB | oriT, KanR |
| pSL439 | Donor | H1, H4 | HygR-SacB | oriT, KanR |
| pSL1071 | Donor | H1, H4 | NsrR-PheS | oriT, KanR |

Construction of barcoded donor plasmids: The donor vector was constructed using standard cloning methods. It contains 1) KanR (kanamycin resistance), 2) oriT (origin of transfer), 3) R6K oriγ (conditional replication origin depending on the phage-derived pir expression), and 4) swapping region, a I-SceI-H1-H4-I-SceI configuration, where I-SceI is the recognition site of the endonuclease SceI, and H1 (5′-ttgccctctctcttcattcagggtcatgagaggcacgccattcaaggggagaagtgagatc-3′(SEQ ID NO: 43)) and H4 (5′-aagaacttttctatttctgggtaggcatcatcaggagcagga-3′ (SEQ ID NO: 44)) are the homology regions for recombination. In the swapping region of donor vectors, a selection cassette (HygR-SacB or NsrR-PheS) was cloned between H1 and H4 to generate donor backbone plasmids for parsing. To insert random barcodes into donor backbones (pSL438 and pSL439), an oligonucleotide (pXL633) that contains a NotI restriction site, a barcode region including a random 15 nucleotides, and a region of homology to both donor backbones, was ordered from IDT. pXL633, paired with pXL585, was used to PCR the barcodes with ˜1 ng of either pSL438 or pSL439 as template. The resulting PCR products were restriction digested and ligated into the corresponding donor vector via NotI and XmaI sites. Following the same cloning protocol above, the ligation products were transformed into competent donor cells BUN20 and the barcoded donor clones were selected on the LB agar plates containing 50 μg/ml kanamycin (Kan) in 37° C. Transformants were then randomly selected and arrayed to generate two 96-well barcoded donor collections: pSL438_BC and pSL439_BC. To identify the barcode sequences in the arrayed donor collections, the regions containing the barcodes were amplified by colony touch PCR using pXL583 and pXL584 as primers. The amplicons were then purified and Sanger sequenced using pXL583. Barcodes were then extracted to compile two lists of known donor barcode collections.

Construction of barcoded recipient plasmids: Plasmid pSL937, which is used as the backbone to insert the random barcodes to generate the arrayed and barcoded recipient collection, were constructed from the following sources by standard methods: 1) plasmid backbone/origin of replication from pBR322, 2) GmR (gentamicin resistance marker) from pUC18-mini-Tn7T-Gm³, 3) homology sequences H1 and H4, and two I-SceI recognition sites in a H1-I-SceI-I-SceI-H4 configuration, 4) a rhamnose-inducible toxin relE (P_{rhaBAD}-relE) from pSLC-2174 was cloned between two SceI sites. Oligonucleotides containing random barcodes were synthesized by IDT and inserted into pSL937 via restriction digestion and ligation.

To insert random barcodes into the recipient backbone (pSL937), an oligonucleotide (pXL631) that contains an XhoI restriction site, a barcode region including 20 random nucleotides, and a region of homology to pSL937, were ordered from IDT. pXL631, paired with pXL154, was used to generate barcodes via PCR with ˜1 ng pSL937 as template. The resulting PCR products were digested and ligated into pSL937 using MluI and XhoI restriction sites. The ligation reactions were performed with 3:1 molar ratio between barcode insert and vector for overnight in 16° C. The ligation products were then transformed into competent BUN21 cells that contain a spectinomycin-resistant helper plasmid pML104¹. Barcoded recipient clones were selected on the LB agar plates containing 50 μg/ml spectinomycin (Sp), 20 μg/ml gentamicin (Gm), and 2% Glucose in 30° C. Transformants were then randomly selected and arrayed into 96-well plates. Barcode sequences at each position in the arrayed recipient collections were identified by sequencing. A total of 841 barcodes could be confidently identified. These barcodes were re-arrayed into 8 new 96-well plates such that there was a unique barcode at each position.

Arrayed mating: Each barcoded donor plate (two 96-position plates) was mated to each barcoded recipient plate (8 96-position plates). The donor barcode collections were grown on LB+Kan plates overnight in 37° C.; the recipient arrays were grown on LB+Sp+Gm+2% Glucose overnight in 30° C. The agar media for arrayed mating contained 0.2% arabinose (Ara) and 0.1 mM IPTG, and were pre-warmed in 37° C. for 1 hour. Both donor and recipient clones were transferred onto the mating plates using SINGER ROTOR HDA pin pads and grown for −3 hours at 37° C. Each recipient plate was mated with two donor barcoded plates (pSL438_BC and pSL439_BC). The mated cells were then transferred onto the selection LB plates that contain 0.2% arabinose, 0.2% rhamnose (Rha), 25 μg/ml gentamicin, and 50 μg/ml hygromycin (Hyg) (LB+Ara+Rha+Gm+Hyg). Recombinant clones were then selected at 37° C. overnight.

Amplicon sequencing: To extract the recombinant plasmids, cells were scraped from the selection plates and mini prepped using Plasmid Plus Mini Kit (QIAGEN). The plasmid DNA was quantified and diluted to ˜1 ng/l, which is approximately 1.5×10⁶ copies of each unique barcode-barcode pair per 96-array plate. A two-step PCR was performed. First, a 4 to 5 cycles of PCR with OneTaq polymerase (New England Biolabs) was performed using the forward (pBPS_fwr) and reverse (pBPS_rev) primers listed in Table 1. ˜1 ng of recombinant plasmid DNA was amplified in a single 50 μl PCR reaction. To increase the multiplexing of sequencing samples, a unique pair of 1st PCR and 2nd PCR primers (see Table 1 and 2) were used to amplify the plasmid DNA from a particular pair of mated plates, which enables pooling of multiple mated plates together in one sequencing library. The cycle conditions for the first step is the following Table 6:

TABLE 6
Cycle conditions

| Cycle | Temperature | Time | |
|---|---|---|---|
| 1 x | 94° C. | 10 | sec |
| 3 x | 94° C. | 15 | sec |
| | 55° C. | 20 | sec |
| | 68° C. | 20 | sec |
| 1 x | 68° C. | 5 | min |

Primers for the first step PCR have this general configuration:

pBPS_fwr:
(SEQ ID NO: 45)
ACACTCTTTCCCTACACGACGCTCTTCCGATCTNNNNNNNNXXXXXXttc
ggttagagcggatgtg
pBPS_rev:
(SEQ ID NO: 46)
GTGACTGGAGTTCAGACGTGTGCTCTTCCGATCNNNNNNNNXXXXXXXXX
aggtaacccatatgcatggc.

The Ns in these sequences correspond to any random nucleotide and are used in the downstream analysis to remove skew in the counts caused by PCR jack-potting. The Xs correspond to a one of several multiplexing tags, which allows different samples to be distinguished when loaded on the same sequencing flow cell. The lowercase sequences correspond to the priming sites on the recombinant plasmids. The uppercase sequences correspond to the Illumina Read 1 or Read 2 sequencing primer. The PCR products were purified using NucleoSpin columns (Macherey-Nagel) and eluted into 33 μl water. A second 23-25 cycles PCR was performed with PrimeStar HS polymerase (Takara), with 33 μl of cleaned product from the first PCR as template and 50 μl total volume per tube. Primers for this reaction were the standard Illumina TruSeq dual-indexed primers (D501-D508 and D701-D712) listed in Tables 1 and 2. The cycle conditions for the second step is the following in Table 7:

TABLE 7
Cycle conditions for the second step

| Cycle | Temperature | Time | |
|---|---|---|---|
| 1 x | 98° C. | 3 | min |
| 23 x | 98° C. | 10 | sec |
| | 69° C. | 5 | sec |
| | 72° C. | 20 | sec |
| 1 x | 72° C. | 1 | min |

PCR products were then cleaned using NucleoSpin columns. Amplicons from each mating plate were uniquely labeled with the customized primer indexes (first PCR) as well as standard Illumina indices (second PCR). This quadruple-indexed strategy increases the multiplexing capacity for sequencing. Cleaned amplicons were pooled and paired end sequenced at ˜800 reads per barcode-barcode pair on an Illumina MiSeq, HiSeq or NextSeq with 25% PhiX genomic DNA spike-in.

Sequencing analysis: Donor-recipient double barcode amplicon sequencing data was analyzed by customized Python scripts and Bartender using the following steps. First, Illumina reads were demultiplexed using the Illumina indices. Any sequences without an exact match to two Illumina indices were discarded. Barcodes were extracted from demultiplexed sequences using the regular expressions
\D*?(.GGC|T.GC|TG.C|TGG.)\D{4,7}?AA\D{4,7}?TT\D{4,7}?(.CGG|G.GG|GC.G|GCG.)\D*” (donor barcode) and
\D*?(.ACA|G.CA|GA.A|GAC.)\D{4,7}?AA\D{4,7}?AA\D{4,7}?TT\D{4,7}?(.TCG|C.CG| CT.G|CTC.)\D*” (recipient barcode). Unique molecular identifiers (UMIs, the Ns in pBPS_fwr and pBPS_rev) were also extracted based on their expected position in the Illumina reads. Barcode reads, which contain a mix of true barcode sequences and sequences that contain errors stemming from PCR or sequencing, were next clustered into consensus sequences using Bartender. Each barcode cluster was next examined for replicate UMIs (indicating PCR duplicates) using Bartender, and all duplicates were removed to generate final counts of each barcode pair. The double barcodes with less than 20 reads were excluded, many of which are expected to be PCR chimeras (barcodes fused by PCR amplification). The remaining reads were used to ascertain the position of each donor barcode from each corresponding recipient barcode.

Whole plasmid sequencing on the Oxford Nanopore platform: Recombinant plasmids containing positioning barcodes and oligonucleotides were extracted as previously described in the amplicon sequencing section. Circular plasmids were linearized by restriction enzyme PmlI (NEB) at 37° C. for 2 hours. Linearized products were size selected by running a 1.2% Agarose gel and recovered using Zymoclean Gel DNA Recovery Kit (Zymoresearch). The ligation sequencing kit (SQK-LSK110, Nanoporetech) was used to construct sequencing libraries for the Oxford Nanopore platform. 300 ng (˜100 fmol) of a linearized recombinant plasmid library was end-repaired using the NEBNext FFPE Repair Mix and NEBNext Ultra II End repair/dA-tailing Module (NEB). Nanopore sequencing adapters (AMX-F) were ligated by NEBNext Quick T4 DNA Ligase (NEB). 30 ng (˜10 fmol) of the library was loaded to a Flongle flow cell (R9.4.1, Oxford Nanopore) to generate reads for recombinant plasmids. The flow cell was run for 16 hours using Miniknow sequencer control software (Version: 21.11.7, Oxford Nanopore).

Oxford Nanopore sequencing analysis; (2) Sequencing adapters were identified and removed and files separated by sample-multiplexing barcodes using “guppy_barcoder” from Guppy version 6.0.1+652ffd179; (3) Alignment to query contaminant sequences (sequence of the origin of replication or transfer for the donor and/or helper plasmids) using “minimap2” version 2.22-ri110-dirty was used to remove unwanted sequence that aligned to these contaminants; (4) Alignments to the expected backbone sequences of the recombined recipient plasmid were generated using “minimap2” version 2.22-ri110-dirty, and a custom python script was used to trim the identical backbone sequence from each read; (5) Position-specific barcodes were extracted from this sequence using fuzzy regular expressions for sequence surrounding the barcode using “itermae” version 0.6.0.1, then clustered using the message-passing Levenshtein-distance approach in “starcode” version 1.4; (6) These barcodes were used to separate the plasmid-backbone-removed sequences into separate files for each demultiplexed sample and clustered barcode sequence, using custom shell/awk scripts; (7) The sequences for each barcode in each sample were used to generate a multiple-sequence alignment using “kalign3” version 3.3.1; (8) A custom python script was used to generate a singular draft consensus sequence from the multiple-sequence alignment by a process of voting; (9) This draft consensus sequence was polished by using “racon” version 1.5.0 to update the consensus based on the read agreements and sequence qualities; (10) This consensus sequence was further polished using “medaka” version 1.5.0 to generate a polished sequence per each positioning barcode in each sample; (11) The payload sequence that is the intended target of the rearraying project was extracted from the polished region again using “itermae” version 0.6.0.1 with different regular expressions. The polished and positioned payloads were analyzed by aligning the raw backbone-sequence-removed regions to the polished regions, by aligning the payload extracted from the polished regions to the intended target sequences, and by aligning the raw backbone-sequence-removed regions to all polished regions generated in the dataset. Alignments were done with “minimap” version 2.22-ri110-dirty or a custom python script using the BioPython PairwiseAlignment functionality. A custom R script was used to identify reads as “on-target”: those having >90% identity to the polished region generated for that sample and barcode (i.e. well). Wells were classified as “pure” if >90% of the raw reads were “on-target” to the polished consensus sequence. “Sequences were compared to the polished payload's length and alignment to an intended target sequence, and defined as “correct” if the polished payload was perfectly identical to one of the intended target sequences.

Construction of donor plasmid libraries containing oligonucleotide pools: Plasmid pSL1071, which contains the NsrR-PheS cassette, two I-SceI sites, and two homology regions for recombination (H1 and H4), was used as the backbone into which to insert the oligonucleotide pool. An oligonucleotide pool containing one-handed 300 bp oligonucleotides was ordered from IDT according to the design:

(SEQ ID NO: 47)
GCTTATTCGTGCCGTGTTATGGCGCGCCNN...NNGCGGCCGCGGGCACA
GCAATCAAAAGTA,

where GCTTATTCGTGCCGTGTTAT and GGGCACAGCAATCAAAAGTA (SEQ ID NO: 48) are priming sites for the forward and reverse primers to amplify the oligonucleotide pool; GGCGCGCC (SEQ ID NO: 49) and GCGGCCGC (SEQ ID NO: 50) are recognition sites for restriction enzymes AscI and NotI; and NN . . . NN denotes the 244-nt sequences that are randomly selected from Human genome assembly GRCh38. Amplification of the oligonucleotide pool was performed with 7 ng of template DNA and KAPA HiFi polymerase (Roche) using cycle conditions described in Table 8.

TABLE 8
Cycle conditions

| Cycle | Temperature | Time | |
|---|---|---|---|
| 1 x | 95° C. | 3 | min |
| 14 x | 98° C. | 20 | sec |
| | 53° C. | 15 | sec |
| | 72° C. | 15 | sec |
| 1 x | 72° C. | 1 | min |

PCR products were purified using DNA Clean & Concentrator-5 (Zymoresearch). To clone PCR products into the donor plasmid pSL1071, AscI and NotI restriction enzyme recognition sites were used. The digestion reaction of PCR products and pSL1071 were performed at 37° C. for 4 hours. Digested products were then size selected by running a 1.2% Agarose gel and recovered using Zymoclean Gel DNA Recovery Kit (Zymoresearch). The ligation reaction was performed with 25 ng of digested vectors and 3.8 ng of inserts using T4 DNA ligase (NEB) at 16° C. for 15 hours. Ligation products were transformed into BUN20, and conjugated to arrays of barcoded recipient plasmids (described above) to determine the sequence of the construct at each position in the donor array.

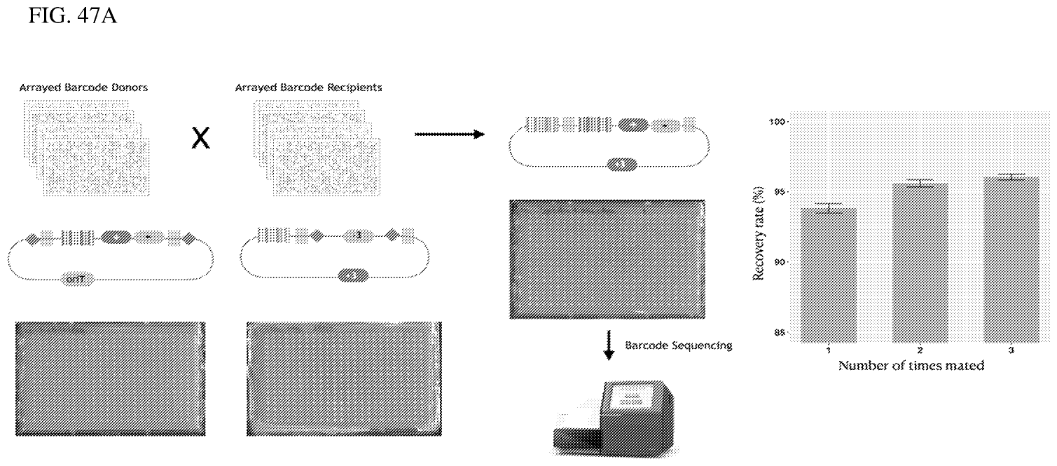

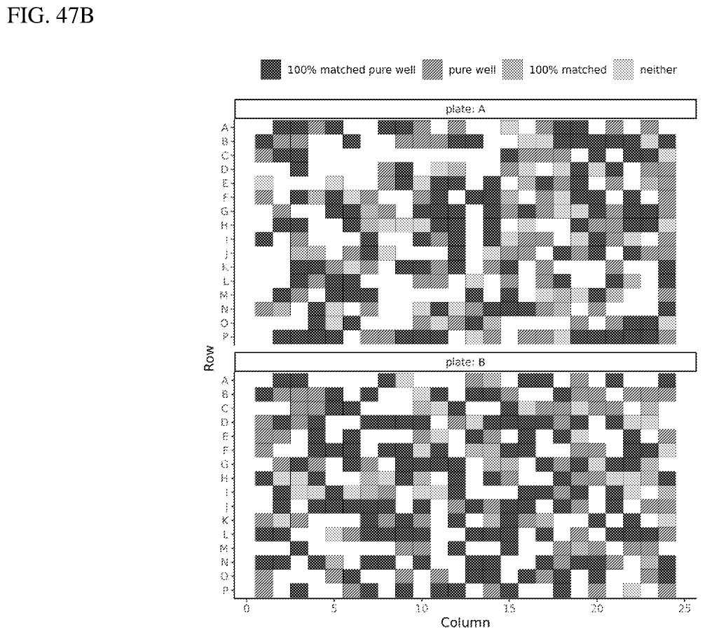

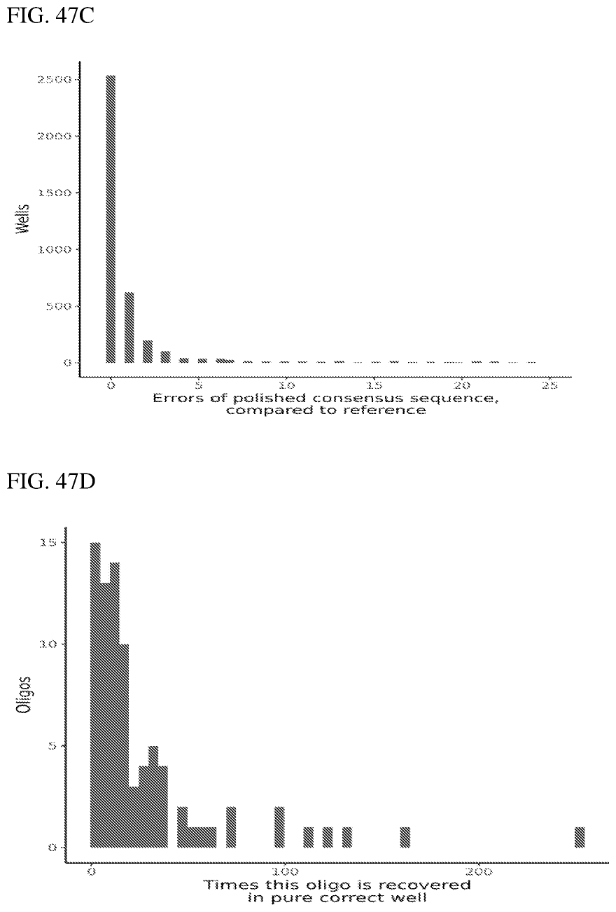

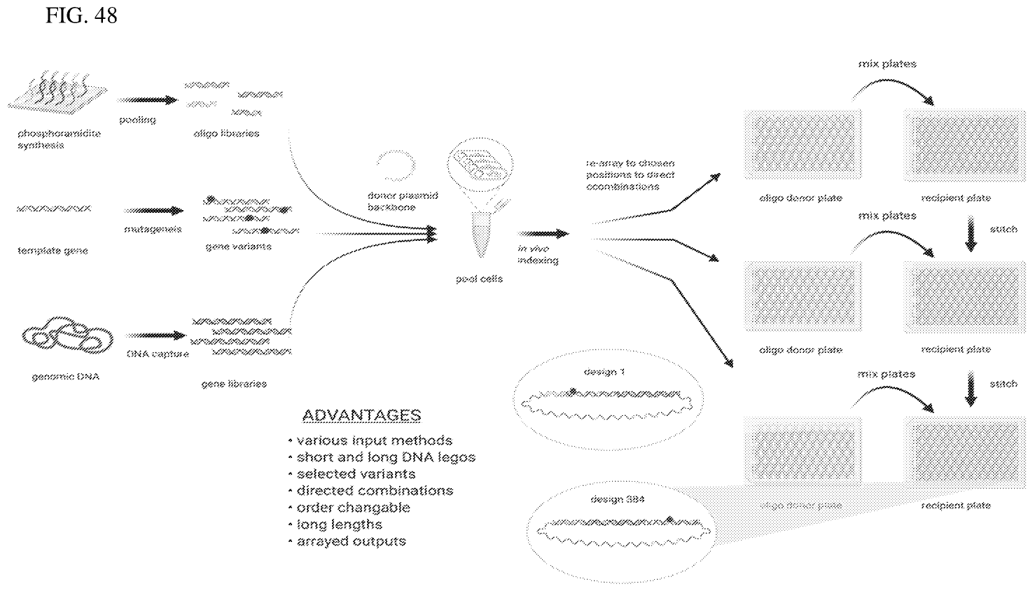

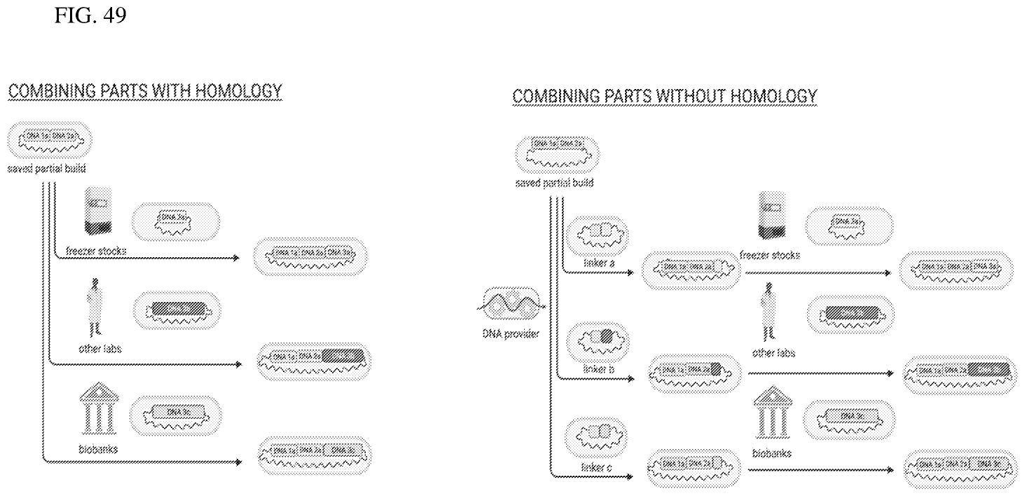

Results: Positioning of barcode arrays. To validate the accuracy of parsing and positioning, each of the two known donor barcode 96-well plates (pSL438_BC and pSL439_BC) was mated to 8 96-well recipient plates. Using the data from these 1536 mating events, it was found that the correct position could be identify and sequence verify 93.82%±0.34%, 95.59%±0.27%, and 96.04%±0.21% of donors using 1, 2, and 3 events, respectively (FIGS. 47A-47D). All misses were due to a lack of sequencing data. An incorrect position was never identified for a donor in the sequencing data. Similar results were found when determining the position of recipient barcodes from donor barcodes.

### Results: Parsing of an Oligonucleotide Pool

To further validate the accuracy of parsing, we arrayed and sequence-verified a pool of 100 oligonucletodies. This pool contained 244-nucleotide sequences randomly selected from the human genome, synthesized as an “oPool” from IDT (Integrated DNA Technologies) and inserted into our donor plasmid pSL1071 using ligation. BUN20 transformants carrying these plasmids were pooled, then randomly arrayed into a total of twenty 384-well plates. These arrayed plates of bacteria were then conjugated to an arrayed collection of recipient barcode strains (barcode positions are known). Recipient cells containing recombinant oligonucleotide-barcode plasmids were pooled. Plasmids were sequenced using Nanopore sequencing. Sequencing results were used to determine for each well in each plate: the consensus sequence of the oligonucleotide, if the consensus sequence is identical to an expected sequence in the oligonucleotide pool, and whether any other oligonucleotide sequences are present at low frequencies (contamination) (FIG. 47B, FIG. 47C, FIG. 47D).Consensus sequences were generated for 5,101 wells out of 7,680 wells (66.4%) available across all plates. Of these consensus sequence wells, 2,329 wells (45.6%) were pure and perfectly matched a target oligo. These 2,329 perfect-match oligos represented 82% of the oligonucleotides expected to be in the pool.

1. A method of assembling a plurality of DNA elements into an assembled DNA element within a recipient cell, the method comprising:
(a) contacting a first donor cell comprising a first donor plasmid with a recipient cell comprising a recipient oligonucleotide under conditions to (i) transfer the first donor plasmid from the first donor cell to the recipient cell by conjugation and (ii) recombine the first donor plasmid and the recipient oligonucleotide in the recipient cell by homologous recombination
wherein the recipient oligonucleotide is in a recipient cell plasmid or the recipient cell genome, and wherein
the first donor plasmid comprises, in sequential order, a first endonuclease site (C1), a first homologous recombination region (HR1), a first oligonucleotide comprising a first DNA element fragment (oligo1), a second homologous recombination region (HR2) comprising two homologous recombination regions (HR2.1, HR2.2) and a third endonuclease site (C3);
the recipient oligonucleotide comprises a third homologous recombination region (HR3) homologous to HR1 and a fourth homologous recombination region (HR4) homologous to HR2.2;
thereby providing, following the homologous recombination of HR1 with HR3 and HR2.2 with HR4, a first recombined recipient oligonucleotide comprising the first DNA element fragment;
(b) contacting a second donor cell comprising a second donor plasmid with the recipient cell comprising the first recombined recipient oligonucleotide under conditions to (i) transfer the second donor plasmid from the second donor cell to the first recipient cell by conjugation and (ii) recombine the second donor plasmid and the first recombined recipient oligonucleotide to form a second recombined recipient oligonucleotide in the recipient cell by homologous recombination wherein
the second donor plasmid comprises, in sequential order, a fifth endonuclease site (C5), a fifth homologous recombination region (HR5) homologous to HR2.1, a second oligonucleotide encoding a second DNA element fragment (oligo2), a sixth homologous recombination region (HR6) comprising two homologous recombination regions (HR6.1, HR6.2), and a sixth endonuclease site (C6);
thereby providing, following the homologous recombination of HR5 with HR2.1 and HR6.2 with HR4, a second recombined recipient oligonucleotide comprising the first and second DNA element fragments (oligo1, oligo2), which form a DNA assembly.

2. The method of claim 1, wherein step (b) is repeated for one or more iterations with a third or subsequent donor cell comprising a third or subsequent donor plasmid comprising compatible HR regions and a third or subsequent oligonucleotide encoding a third or subsequent DNA element fragment (oligo3, oligo4, . . . oligoN), thereby forming a third or subsequent recombined recipient oligonucleotide comprising the first, the second, and a third or subsequent DNA element fragments which together form a DNA assembly.

3. The method of claim 1, wherein step (a) comprises a plurality of first donor cells, each comprising a different first donor plasmid; and step (b) comprises a plurality of second, third, or subsequent donor cells, each comprising a different second, third, or subsequent donor plasmid; optionally wherein each first donor cell is in a position in a first ordered array and each second, third, or subsequent donor cell is in a position in a second, third, or subsequent ordered array.

4. The method of claim 1, wherein an oligonucleotide encoding a guide RNA or a first endonuclease targeting the first, third, and/or fourth endonuclease sites is present on the first donor plasmid.

5. The method of claim 4, wherein an oligonucleotide encoding a guide RNA or a second endonuclease targeting the second, fifth, and/or sixth endonuclease site is present on the second donor plasmid.

6. The method of claim 4, wherein expression of the first and/or the second endonuclease is inducible and the method further comprises inducing expression of the first and/or second endonuclease.

7. The method of claim 5, wherein the first and/or the second endonuclease is selected from an RNA-guided endonuclease, a homing endonuclease, a transcription activator-like effector nuclease, and a zinc finger nuclease.

8. The method of claim 1, wherein the first, second or subsequent donor plasmid comprises a selectable marker selecting for integration of the first oligonucleotide, second oligonucleotide or subsequent oligonucleotide into the recipient oligonucleotide.

9. The method of claim 1, wherein the recipient oligonucleotide comprises a counter-selectable marker selecting against recipient cells that do not comprise the first, second, third, or subsequent oligonucleotide.

10. (canceled)

11. The method of claim 1, wherein the donor plasmid comprises a conditional replication origin.

12. The method of claim 1, wherein the donor plasmid or recipient oligonucleotide comprises an inducible high-copy replication origin.

13. The method of claim 1, wherein the donor plasmid or recipient oligonucleotide comprises a replicon that can replicate plasmids of lengths greater than 30 kilobases.

14-16. (canceled)

17. The method of claim 1, wherein the donor plasmid or the recipient cell comprises an oligonucleotide encoding one or more homologous DNA repair genes; optionally wherein expression of the one or more homologous DNA repair genes is inducible.

18. The method of claim 1, wherein the donor plasmid or recipient cell comprises an oligonucleotide encoding one or more recombination-mediated genetic engineering genes.

19. (canceled)

20. The method of claim 1, wherein the assembled DNA element is from 100 nucleotides to 500,000 nucleotides in length.

21. The method of claim 1, wherein the first, second or subsequent homologous recombination (HR) regions and their corresponding HR regions on the recipient oligonucleotide each comprise from about 20 base pairs to about 500 base pairs; optionally about 50 to 100 base pairs.

22-30. (canceled)

31. The method of claim 1, wherein the method comprises utilizing two or more recipient oligonucleotides having compatible homologous recombination regions to construct a DNA library.

32. (canceled)

33. (canceled)

34. The method of claim 1, wherein the method is used to assemble a mutagenesis library; to combine genetic regions such as genes, promoters, terminators, and regulatory regions from different species; to construct and/or combine genetic regulatory pathways; to construct combinatorial gRNA libraries; or to assemble arrays of bacteria containing plasmids for screening assays.

35. The method of claim 1, wherein prior to steps (a) and (b) of claim 1, the first and second oligonucleotides comprising the first and second DNA element fragments are inserted into the first and second donor plasmids.

36-65. (canceled)

66. The method of claim 1, wherein HR2.1 and HR2.2 flank a non-homologous region comprising one (C2) or two endonuclease sites (C2.1, C2.2);
HR3 and HR4 flank a non-homologous region comprising one (C4) or two endonuclease sites (C4.1, C4.2); and/or
HR6.1 and HR6.2 flank a non-homologous region comprising one (C7) or two endonuclease sites (C7.1, C7.2).

67. The method of claim 1, wherein the DNA assembly comprises at least a portion of a gene, a promoter, an enhancer, a terminator, an intron, an intergenic region, a barcode, a guide RNA (gRNA), or a combination thereof.
